SEQUENCE LISTING (87 sequences)

SEQ ID NO: 1 - protein, 1298 aa, Thiomicrospira sp.
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 2 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 3 - protein, 1333 aa, Catenibacterium sp.
MSQNIVDYCIGLDLGTGSVGWAVVDMNHRLMKRNGKHLWGSRLFSNAETAANRRASRSIRRRYNKRRERIRLLRAILQDMVLENDPTFFIRLEHTSFLDEEDKANYLGADYKDNYNLFIDEDFNDYTYYHKYPTIYHLRKALCESTEKADPRLIYLALHHIVKYRGNFLYEGQKFNMDASNIEDRLSDVFTQFADFNNIPYEDDEKKNLEILEILKKPLSKKAKVDEVMALIAPEKDFKSAYKELVTGIAGNKMNVTKMILCEPIKQGDSEIKLKFSDSNYDDQFSEVENDLGEYVEFIDSLHNIYSWVELQTIMGATHTYNASISEAMVSRYNKHHEDLQLLKKCIKDNVPKKYFDMFRNDSEKLKGYYNYINHPSKAPVDEFYKYVKKCIEKVDTPEAKQILHDIELENFLLKQNSRTNGSVPYQMQLDEMIKIIDNQAKYYPVLKEKREQLLSILTFKIPYYFGPLNETSEHAWIKRLEGKENQRILPWNYQDIVDVDATAEGFIKRMRSYCTYFPDEEVLPKNSLIVSKYEVYNELNKIRVDDKLLEVDIKNDIYNELFMNNKTVTEKKLKNWLVNNQCCNKNAEIKGFQKENQFSTSLTPWIDFTNIFGEINQSNFDLIEDIIYDLTVFEDKKIMKRRLKKKYALPDDKIKQILKLKYKDWSRLSKKLLDGIVADNKFGSSVTVLDVLEMSRLNLMEIINDRDLGYAQMIEAAASCPEDGKFTYKEVQRLAGSPALKRGIWQSLQIVEEITKVMKCRPKYIYIEFERSEETKERTESKIKKLENVYKDLDEQTKVEYKTVLEELKGFDNTKKISSDSLFLYFTQLGKCMYSGKKLDIDSLDKYQIDHIVPQSLVKDDSFDNRVLVVPSENQRKLDDLVVPSDIRVKMNSFWKLLFDHELISPKKFYSLIKTEYTERDEERFINRQLVETRQITKNVTQIIEDHYSTTKVAAIRANLSHEFRVKNHIYKNRDINDYHHAHDAYIVALIGGFMRDRYPNMHDSKAVYSEYMKMFRKNKNDKKRWKDGFVINSMNYPYEVDGELIWNPDIINEIRKCFYYKDCYCTTKLDQKSGQMFNLTVLPNDAHSPKGTTEAVIPVNKNRKDVNKYGGFSGLQYVIVAIEGKKKRGKKTKLVKKISGVPLHLKAASLDEKIKYIEEKENLTDVKIIKDSIPVNQMIEMDGGEYLLTSPIEFVNGRQLVLNEKQCALIADIYNAIYKQDCDNLDDVLMIQLYIELINKMKALYPAYQSIAEKFESMTEDYVAVSKEEKADIIKQMLIIMHRGPRNGKIQYADFNVGDRIGRKNKMSLDLERVTFVSQSPTGIYTKKYKL

SEQ ID NO: 4 - protein, 1344 aa, Kandleria vitulina
MSQNNNKIYNIGLDIGDASVGWAVVDEHYNLLKRHGKHMWGSRLFTQANTAVERRSSRSTRRRYNKRRERIRLLREIMEDMVLDVDPTFFIRLANVSFLDQEDKKDYLKENYHSNYNLFIDKDFNDKTYYDKYPTIYHLRKHLCESKEKEDPRLIYLALHHIVKYRGNFLYEGQKFSMDVSNIEDKMIDVLRQFNEINLFEYVEDRKKIDEVLNVLKEPLSKKHKAEKAFALFDTTKDNKAAYKELCAALAGNKFNVTKMLKEAELHDEDEKDISFKFSDATFDDAFVEKQPLLGDCVEFIDLLHDIYSWVELQNILGSAHTSEPSISAAMIQRYEDHKNDLKLLKDVIRKYLPKKYFEVFRDEKSKKNNYCNYINHPSKTPVDEFYKYIKKLIEKIDDPDVKTILNKIELESFMLKQNSRTNGAVPYQMQLDELNKILENQSVYYSDLKDNEDKIRSILTFRIPYYFGPLNITKDRQFDWIIKKEGKENERILPWNANEIVDVDKTADEFIKRMRNFCTYFPDEPVMAKNSLTVSKYEVLNEINKLRINDHLIKRDMKDKMLHTLFMDHKSISANAMKKWLVKNQYFSNTDDIKIEGFQKENACSTSLTPWIDFTKIFGKINESNYDFIEKIIYDVTVFEDKKILRRRLKKEYDLDEEKIKKILKLKYSGWSRLSKKLLSGIKTKYKDSTRTPETVLEVMERTNMNLMQVINDEKLGFKKTIDDANSTSVSGKFSYAEVQELAGSPAIKRGIWQALLIVDEIKKIMKHEPAHVYIEFARNEDEKERKDSFVNQMLKLYKDYDFEDETEKEANKHLKGEDAKSKIRSERLKLYYTQMGKCMYTGKSLDIDRLDTYQVDHIVPQSLLKDDSIDNKVLVLSSENQRKLDDLVIPSSIRNKMYGFWEKLFNNKIISPKKFYSLIKTEFNEKDQERFINRQIVETRQITKHVAQIIDNHYENTKVVTVRADLSHQFRERYHIYKNRDINDFHHAHDAYIATILGTYIGHRFESLDAKYIYGEYKRIFRNQKNKGKEMKKNNDGFILNSMRNIYADKDTGEIVWDPNYIDRIKKCFYYKDCFVTKKLEENNGTFFNVTVLPNDTNSDKDNTLATVPVNKYRSNVNKYGGFSGVNSFIVAIKGKKKKGKKVIEVNKLTGIPLMYKNADEEIKINYLKQAEDLEEVQIGKEILKNQLIEKDGGLYYIVAPTEIINAKQLILNESQTKLVCEIYKAMKYKNYDNLDSEKIIDLYRLLINKMELYYPEYRKQLVKKFEDRYEQLKVISIEEKCNIIKQILATLHCNSSIGKIMYSDFKISTTIGRLNGRTISLDDISFIAESPTGMYSKKYKL

SEQ ID NO: 5 - protein, 1402 aa, Clostridiales bacterium KA00274
MAKKDYTIGLDIGTNSVGWAIIDDNLKLLKRNMTIKGNTDKKSVKRDLWGSLLYSGNSDKTTSAADARSKRGLRRRLRRRKYRLDRLKQIFSEIINDKAPNFFDKLNESFLNPKDKKYGKYQIFDTEKEEKDYYRRYPTIYHLRKDLIESSKKQDIRLVYLALAHILKSRGNFLFEGNIDDLKNDFAGIYEEVVELCMTINAEDVDLEFEEVDKQSLNSIIKNEDISEIEQGLENFADEHVIFKEQNKKKNDLFSNCCKIICGHTVKANKFASELDSELFISFKSDDYVDVIDVIQSGNENIANLLLACRKAYDYIMFNRLVDLNIDSPAKLSSNMVSLYNQHEKDLKAYKKLIKEFNKFKRSNGCKDLEMIILTADDIDSFRKKVDKKEGKLNGINKKITHEQALKKQLKDMKKILEDKNTEAEDKQINDILKMITSIEERVNKSCFLKNLRSTDNASIPNQIQRQEMEAILDKQAKFYPFLNEHKDELLQLLSFRIPYYVGPLVNKKYSRFAWLVRKEGQVQKITPTNFDGVVDKHKTAEKFMERLIGKDVYLPNERVLPKASLLYQEYCIFNELTKVAYIDSTGKKNNFSSEEKLNIFEKLFKTKREVTKTDLCKCLNNVCKLKEKVKETEIIGIKAKFNAKYSTYHDLKKINGMEQLIADEEGKPLCEDIISILTIFEDKDIRLVRLKELLCQNKDLINKFSLSAEKLAKVLSTKHYKGFGNVSAKLINGIRDKNCKTILDYLIEDDKEAYYGRNNPNRNLMQLVNDSRLAFKGQIDREQNTHLEDLSLDEFLDDLYVSPSIRRGIRLTIRLVDELVEIMGYLPKNIVIEMPREDGEKGKIADTRYSKLEKMLKKDAALEDLYRVLKTYEKNKKALANDALYLYFLQNGRDMYTGKEINLSELHSYDIDHIIPKSFKYDDSLDNKVLTAKKMNMDKRTGALDHNIIENQCGFWRVLLQQDKISLEKYTNLMKTEFTEADKAGFIMRQLVETRQITKFVARYLDNKFNGLISDPNDKVNILLPRASLCHQFRETFGFYKVRELNDMHHAHDAYLNAVIANTLNKNAYLSDLLKYGAYSKYKKNGFNNSNGIMDYFGNTQFNCLFVVERTLDKCRVNIVKHPETASGEFYNETIQKNKVNGGSSTRSLKSSVKVLQNTEQYGGFTNVNNAYFILFDYKAKSKLKRKLIGVPIVDRQKFEQDPVTYLEAKGFDEPKLVQKLLKYTLLEYEDGKRRYLTGVTGKRCELVRANQLLLPRNMMALLHHLQEWQKHDFGIKEMTKVIKNTNNIEAKFDKLFEHMMKFIDKYSEPPKIVSSKISEEYHKLRESLCQDDNKIKIYAEIGKALLSLLHLVDSKSACVFKFSGLEINRIRYQSINEKKEPVIIFQSLSGLRESRYKYNQ

SEQ ID NO: 6 - protein, 1338 aa, Lachnospiraceae bacterium 3-2
MRDYYIGLDLGTGSLGWAVTDREYEIMRAHGKALWGVRLFDSANTAEERRGFRTARRRLDRRNWRIELLQELFGEDIGKVDSGFFLRMKESKYMPEDKRDVNGNCPKLPYALFVEDGYTDKDYHRQFPTIYHLRKWLMETEETPDIRLVYLALHHMMKHRGHFLFSGNIEKIKEFQETFRQYIGKIREEELDFHLCIEGEELRETENILKDKNLTRSAKKTRLIKLLGAHTACEKAALNLVAGGTVKLSDIFGNSELDACEKPKLSFADAGYDDYAGMIEDELGEQHVIIETAKAVYDWSVLADILGDYRCISEAKAAVYEKHQKDLRHLKELVKENLGRDVYKEVFVKTNEKLPNYSAYIGMTKKNGVKSEMEGKRCDRKAFYDYLKKTVVNAIPDESKTEYLRKEMETETFLPRQVTKDNGVIPHQVHLQELDAILENLSGRIPALKENGSKIRDIFTFRIPYYVGPLNGIVKGGERTNWVRRKKAGRICPWNFDEMVDTGASAEEFIRRMTSKCTYLIHEDVLPKNSMLYSKFMVLNELNNVRLNGEPISVELKQKTYEDLFQRHRKVTRRRLTDYIRREGIAGRDADITGIDGDFKGSLTAYHDFKEKLTGCELSQADKENIILNITLFGEDKALLKKRLGALYPALTEPQKKAICALSYKGWGRLSQRLLEGITAPAPETGEIWTVIRAMWETNDNLMQVLSEKYCFAAAIDEENAGEELKEITYKTVEQMNVSPAVRRQIWQSLQVIKEICKVMGGPPKRVFVEMAREKMESKRTESRKKRLIDLYKKCREEERDWIEELGNTEETRLRSDKLYLYYTQKGRCMYSGEVIELEELWDNRKYDIDHIYPQSKVMDDSLDNRVLVKKEYNADKTDEYPIRADIRGKMRAFWRILREEGFISKEKYNRLTRGTGFEPSELAGFIARQLVETRQGTKAVASVLKQVFPETDIVYAKARVASQFRQEFDLIKVREMNDLHHAKDAYVNIVVGNVYYTKFTSNAAWYVKEHPGRSYNLKKMFTSERDVARNGETAWRAGNSGTIATVKRVMGKNNILVTRRSYEVKGGLFDQQLMKKGKGQVPIKGRDERLADIDKYGGYNKAAGTYFMLAESEDKKGAKIRSVEYVPLYLCNCIEKDEEAAKKYLQKERGLKNPRVLIAKIKIDTLFKVDGFYMWLSGRTGNQLIFKGANQLILSEPDMRILKKVLKYVNRKKENKNAVLGEHDQLPETDLIRLYDVFLDKIENTVYHVRLSAQQGTLTKNKDTFCELSNEDKCIVLSEILHMFQCQSGSANLKLIKGPGSAGILVLNNIISKCNQVSIIHQSPTGIYEQEIDLKKI

SEQ ID NO: 7 - protein, 1340 aa, Dorea longicatena
MEQEYYLGLDMGTGSVGWAVTDSEYHVLRKHGKALWGVRLFESASTAEERRMFRTSRRRLDRRNWRIEILQEIFAEEISKKDPGFFLRMKESKYYPEDKRDINGNCPELPYALFVDDDFTDKDYHKKFPTIYHLRKMLMNTEETPDIRLVYLAIHHMMKHRGHFLLSGDINEIKEFGTTFSKLLENIKNEELDWNLELGKEEYAVVESILKDNMLNRSTKKTRLIKALKAKSICEKAVLNLLAGGTVKLSDIFGLEELNETERPKISFADNGYDDYIGEVENELGEQFYIIETAKAVYDWAVLVEILGKYTSISEAKVATYEKHKSDLQFLKKIVRKYLTKEEYKDIFVSTSDKLKNYSAYIGMTKINGKKVDLQSKRCSKEEFYDFIKKNVLKKLEGQPEYEYLKEELERETFLPKQVNRDNGVIPYQIHLYELKKILGNLRDKIDLIKENEDKLVQLFEFRIPYYVGPLNKIDDGKEGKFTWAVRKSNEKIYPWNFENVVDIEASAEKFIRRMTNKCTYLMGEDVLPKDSLLYSKYMVLNELNNVKLDGEKLSVELKQRLYTDVFCKYRKVTVKKIKNYLKCEGIISGNVEITGIDGDFKASLTAYHDFKEILTGTELAKKDKENIITNIVLFGDDKKLLKKRLNRLYPQITPNQLKKICALSYTGWGRFSKKFLEEITAPDPETGEVWNIITALWESNNNLMQLLSNEYRFMEEVETYNMGKQTKTLSYETVENMYVSPSVKRQIWQTLKIVKELEKVMKESPKRVFIEMAREKQESKRTESRKKQLIDLYKACKNEEKDWVKELGDQEEQKLRSDKLYLYYTQKGRCMYSGEVIELKDLWDNTKYDIDHIYPQSKTMDDSLNNRVLVKKKYNATKSDKYPLNENIRHERKGFWKSLLDGGFISKEKYERLIRNTELSPEELAGFIERQIVETRQSTKAVAEILKQVFPESEIVYVKAGTVSRFRKDFELLKVREVNDLHHAKDAYLNIVVGNSYYVKFTKNASWFIKENPGRTYNLKKMFTSGWNIERNGEVAWEVGKKGTIVTVKQIMNKNNILVTRQVHEAKGGLFDQQIMKKGKGQIAIKETDERLASIEKYGGYNKAAGAYFMLVESKDKKGKTIRTIEFIPLYLKNKIESDESIALNFLEKGRGLKEPKILLKKIKIDTLFDVDGFKMWLSGRTGDRLLFKCANQLILDEKIIVTMKKIVKFIQRRQENRELKLSDKDGIDNEVLMEIYNTFVDKLENTVYRIRLSEQAKTLIDKQKEFERLSLEDKSSTLFEILHIFQCQSSAANLKMIGGPGKAGILVMNNNISKCNKISIINQSPTGIFENEIDLLKI

SEQ ID NO: 8 - protein, 1338 aa, Coprococcus catus
MKQEYFLGLDMGTGSLGWAVTDSTYQVMRKHGKALWGTRLFESASTAEERRMFRTARRRLDRRNWRIQVLQEIFSEEISKVDPGFFLRMKESKYYPEDKRDAEGNCPELPYALFVDDNYTDKNYHKDYPTIYHLRKMLMETTEIPDIRLVYLVLHHMMKHRGHFLLSGDISQIKEFKSTFEQLIQNIQDEELEWHISLDDAAIQFVEHVLKDRNLTRSTKKSRLIKQLNAKSACEKAILNLLSGGTVKLSDIFNNKELDESERPKVSFADSGYDDYIGIVEAELAEQYYIIASAKAVYDWSVLVEILGNSVSISEAKIKVYQKHQADLKTLKKIVRQYMTKEDYKRVFVDTEEKLNNYSAYIGMTKKNGKKVDLKSKQCTQADFYDFLKKNVIKVIDHKEITQEIESEIEKENFLPKQVTKDNGVIPYQVHDYELKKILDNLGTRMPFIKENAEKIQQLFEFRIPYYVGPLNRVDDGKDGKFTWSVRKSDARIYPWNFTEVIDVEASAEKFIRRMTNKCTYLVGEDVLPKDSLVYSKFMVLNELNNLRLNGEKISVELKQRIYEELFCKYRKVTRKKLERYLVIEGIAKKGVEITGIDGDFKASLTAYHDFKERLTDVQLSQRAKEAIVLNVVLFGDDKKLLKQRLSKMYPNLTTGQLKGICSLSYQGWGRLSKTFLEEITVPAPGTGEVWNIMTALWQTNDNLMQLLSRNYGFTNEVEEFNTLKKETDLSYKTVDELYVSPAVKRQIWQTLKVVKEIQKVMGNAPKRVFVEMAREKQEGKRSDSRKKQLVELYRACKNEERDWITELNAQSDQQLRSDKLFLYYIQKGRCMYSGETIQLDELWDNTKYDIDHIYPQSKTMDDSLNNRVLVKKNYNAIKSDTYPLSLDIQKKMMSFWKMLQQQGFITKEKYVRLVRSDELSADELAGFIERQIVETRQSTKAVATILKEALPDTEIVYVKAGNVSNFRQTYELLKVREMNDLHHAKDAYLNIVVGNAYFVKFTKNAAWFIRNNPGRSYNLKRMFEFDIERSGEIAWKAGNKGSIVTVKKVMQKNNILVTRKAYEVKGGLFDQQIMKKGKGQVPIKGNDERLADIEKYGGYNKAAGTYFMLVKSLDKKGKEIRTIEFVPLYLKNQIEINHESAIQYLAQERGLNSPEILLSKIKIDTLFKVDGFKMWLSGRTGNQLIFKGANQLILSHQEAAILKGVVKYVNRKNENKDAKLSERDGMTEEKLLQLYDTFLDKLSNTVYSIRLSAQIKTLTEKRAKFIGLSNEDQCIVLNEILHMFQCQSGSANLKLIGGPGSAGILVMNNNITACKQISVINQSPTGIYEKEIDLIKL

SEQ ID NO: 9 - protein, 1350 aa, Enterococcus columbae
MQQYYLGVDMGSASVGWAVTDEKYQLVRKKGKDLWGVRTFDIAQTAEVRRVSRTNRRRQNRRKQRIQILQELLGEEVLKIDAGFFHRMKESRYVAEDKRTLDGKQVELPYALFVDQGFTDKDFYKQFPTINHLIVYLMTTSDTPDIRLVYLALHYYMKNRGNFLHSGDINDVKDIQSILEQLENVLKEYVDDWELSLKDKVDAIKEIYNKDLGRGERKKAFINTLGVKTKSAKAFCSLISGGSTNLAELFDDSGLKESEYAKIEFANANFEDSVEGIQALLEDRFAVIEAAKRLYDWKILTDILGDNASLAEARVKSYETHHEQLVELKSFIKKYLDRKIYQDIFINPNIANNYPAYVGHTKINGKKQELEVKRAKRNDFYAYIKKQVIDPIKKKVSDKAVLARLAEIESLIEVNKYLPLQVNSDNGVIPYQIKLNELRRIFNNLENRLPVLKENRDKIIKTFSYRIPYYVGPLNGVNRNGKSTNWMVRKEGEEGKIYPWNFEEKVDLEASAEKFIRRMTNKCTYLVNEDVLPKYSLLYSKYLVLSELNNLRLDGRPLEVSVKQEIYENVFKRNRKVTLKKIKNYLLKEGVISEKDELSGLADDVKSSLTAYHDFKEKLGHLTLTEDQMEKIILNVTLFGDDKKLLKKRLAALYPNIDEKSLSRMATFNYRDWGRLSKKFLSEITSVDQETGELRTIIQCMYETQNNLMQLLSEPYHFVEAIEKENPKVDLESISYRIVNDLYVSPAVKRQIWQTLLVIKDIKQVMKHDPKRIFIEMAREKQESKTTKSRKQVLSEVYKNAEKYKNLFEKLNSLTEEQLRSKKVYLYFTQLGKCMYTNDAIDFENLVSANSNYDIDHIYPQSKTIDDSFNNLVLVKKGINNDKSDRYPIDKNIRDDEKVKTLWNTLLSKGLITKEKFERLIRSTPFSDEELAGFIARQLVETRQSTKAVAEILSNWFPESEIVYSKAKHITNFRQDFEILKVRELNDCHHAHDAYLNIVVGNAYHTKFTNSPYRFIQNKANQEYNLRKLLQKAKKIESNGVIAWIGQSENNPGTIATVKKVISRNTVLISRMVKEVDGQLFDQQLMKKGKGQVPIKSSDDRLIDISKYGGYNKAKGAYFVFIKSVRRGKTIKSFEYIPVHLAKKFDCNLELLKEYLESEKDLNNVEILMPKVMINSLFNYNGSLIRIPGRYDKKSLLINVDVPLLLESQHIKQLKVIEKYMYKKRVSKNSNILLTKFASDQLKDLDALFDVLSYKLNENIYNVINDKYDKLVICRDKFISLDTEVKCEMIFELLHLFQCNSQLANITKIGATSKFGSISMSKNLKENDKMSIIHQSPSGIFEHEIELTAL

SEQ ID NO: 10 - protein, 1319 aa, Fructobacillus sp.
MGYNIGLDIGTGSVGWAALTDEGKLARAKGKNLIGVRLFDSAQSAAQRRSYRTTRRRLSRRKWRLRLLENIFSDEMGMIDENFFARLKYSYVHPKDEVNNAHYYGGYLFPTQQETHDFHEKFQTIYHLRLKLMIEDCKFDLREIYLAMHHIVKYRGHFLNSQSKMTIGDSYNPRDFQQAIQNYAEAKGLIWSLNDAQEMTDVLVGQAGFGLSKKAKAERLLSAFSFDTKEDKKAIQAILAGIVGNTTDFTKIFNRERSGDELKKWKLKLDSEAFDEQSQAIVDELDDDEMELFNAIRQAFDGFTLMDLLGDQTSISAAMVKRYQQHHDDLKMVKEIAKKQGLSHQDFSKIYTAFLKDDTDKGMKALLDKADLADDVLVEIQQRIESHDFLPKQRTKANSVIPYQLHLAELEKIIENQGKYYPFLLDTFTNKAGETINKLVELVKFRVPYYVGPMVTAADVEKAGGDATNHWVKRNEGYEKSPVTPWNFDQVFNRDQAAQDFIDRLTGTDTYLIGEPTLLKNSLKYQLFTVLNELNNVKINGHKIDEKTKHVLIQDLFKSKKTVSEKAIKDYYLSQGMGEIQIVGLADKTKFNSNLSSYIDLSKTFDAEFMENPANQELLENIIQIQTVFEDVKIAERELQKLALPDEQVQQLAKTHYTGWGNLSDKLLSTPIIQEGSQKVSILNKLQTTSKNFMSIITDNKFGVQQWIQEQNTAETADSIQDRIDELTTAPANKRGIKQAFNVLFDIQKAMGEEPNRVYLEFAKETQNSVRTNSRYNRLKDLYKSKTLSDDVKALKEELESQKSSLQSERIGDRLYLYFLQQGKDMYTGQPINIDKLSTDYDIDHIIPQAYTKDDSIDNRVLVSRPENARKSDSATYTTEVQQSAGGLWKSLKNAGFISQKKYDRLTKGGDYSKGQKTGFIARQLVETRQIIKNVASLIESEFSQTKAVAIRSEITADMRRLVAIKKHREINSFHHAFDALLITAAGQYMQARYPDRDGANVYNEFDYYTNTYLKELRQSSSSSQVRRLKPFGFVVGTMAKGNENWSEDDTQYLRHVMNFKNILTTRRNDKDNGALNKETIYAVDPKAKLIGTNKKRQDVSLYGGYIYPYSAYMTLVRANGKNLLVKVTISAAEKIKSGQIELSEYVQQRPEVKKFEKILINKLAIGQLVNNDGNLIYLTSYEFYHNAKQLWLPTEEADLISQLNKDSSDEDLIKGFDILTSPAILKRFPFYELDLKKLVNIRDKFIAVENKFDILMVILKALQLDAAQQKPVKMIDKKSADWKDYRQRGGIKLSDTSEIIYQSTTGIFEKRVKISNLL

SEQ ID NO: 11 - protein, 1319 aa, Weissella halotolerans
MAYSVGLDIGVGSVGFAGIDNQYNLVRTKGKNVIGVRLFDEADSAAERRGHRTNRRRLQRRRWRLRLLDDIFAKPLQAVDPNFLARLKYSYVNKKDQGQQDHYYGGYVFGSTAADQAYHQAYPTIYHLRKRLMEDDQKHDLREVYLAIHHIVKYRGNFLNPQSSLDIDQQFDVTDFAQALARFADHQALSWALEAPIRFLEAELATGLSNSARVDAAIEAFSFDTKVDRAAIKEMLKGLSGNQIDFTKLFVNVDSADWDQEERKQWKMKLSEEDFDEQALPILERLSQDETEFFLAIKRAYDGIALMRFLGDEQSLSSAMIKAYEDHRRDLTFLKTQVRTPQNRQALSEGYTNYLSVDDKKHKRGAKELAQLIEASDASEQDKATMLDRIANDQFAPKQRTKANGLIPYQLHLAELKKILAKQGQYYPFLLDTFAKQGQSVNKIEELVQFRVPYYVGPMVPKSETAGNAENHWVEKNDGQTKVSVTPWNFDQVFNRDRAAKSFIDRLTGTDTYLIGEPTLPRHSLTYETFTVLNELNNIRIDGKRLPVETKQAIVEDLFKKYRLVTKKRLQDYFASFGKREVELTGLADESRFTSSLTSYHDLQGLLGTDFITNPQNHSLLEKIVEIQTVFEDSDIAERELGKLGLEQKLIPRLAKKHYTGWGNLSRKLLDTSFIHDPERPEEPVSIMDLLYTTNKNFMEILHDSEYGVEEWLKSQNMIDDQKDIQMRIDELTTSPANKRGIKQAFNVLDDITQAMGEEPAYVYLEFAREKQASRRTVSRKKRLETLYKNAALKTEFKAIKEALAEESDDRMQDDRLYLYYAQLGRDMYTGQSISIDQLSSHYDIDHIVPRAFIKDDSLENKVLVNRTDNARKTDSATFTADVKAKAFPLWQQLKKLGLISAKKFRLLTRTGDFTEMERERFIARQLVETRQIIKNVAALIEGHFSQTQAVAIRAEVTGELRQLTQIKKDRDINDYHHAQDALLVATAGTYLHRHFPKRDARFIYNEFDYYTQHWLKNQGENRRRHPYSFVVGTMSKGNEDWTPDNLNYLRKVMQYKTMLMTRKPVGPEGALYKETLIAADPKKRLVGASKERQDPTIYGGYTKESSAYMSLVRAGGKNQLVKIPVRIANEIHSGQRKLDDYVQAKVKKFERILLPKISLGQLVEDEGQRFYLATNEMKHNAKQLWLDQKVVTTYKRLTAESPVEDFLTVFDALTSSATIHHFKFYQRDLELLRDNRAGFQDLAKATQLKVLKDVLYELHDNAGWRDPIKQYFKEIGLKVRMWTKLQKEGGIKLTDQAELIYQSPSGLFEKRRRVQDLL

SEQ ID NO: 12 - protein, 1364 aa, Pediococcus acidilactici
MGDRKYNLGLDIGTSSIGFAAVDENNQPIRVKGKTAIGVRLFEEGKTAADRRGFRTTRRRLSRRRWRINLLNEIFDAHLAEVDPTFLARLKESNRSNLDPKKSFQGSLLFPERKDYQFYEEYPTIYHLRKALMEKDRKFDFREIYLAVHHIIKYRGNFLNGTPMRSFKVENIELDTLFDQLNQLYAEIVPDNELAFDLAQVADVKDVLSSTTIYKMDKKKQLVKMMLLPASNKALQSENKKIVTQFVNAILNYKFKLDVLLQVETDADWSLKLNDEGADDKLEEFTGDLDENRLEIIDLLQRLHNWFSLNEITKDGNSLSAAMVEKYENHHHHLGLLKKVIENHPDAKKAKALKETYTAYVGKTDDKTQNQDDFYKAVEKNLDDSPDAKEIKRLIQLDQFMPKQRTGQNGAIPHQLHQQELDQIIEKQSKYYPFLAEPNPNVKRRKDAPYKLDELIAFKIPYYVGPLVTPEEQAQNGENVFAWMKRKAAGPITPWNFDEKVDRMESANRFIRRMTTKDTYLFGEDVLPAESMIYQKFVVLNELNNLKINGRHLSLKDKQDVYNDLFKQQKTVSIKALQNYYVTKKKAATAPTVGGLADPKKFLSSLSTYIDFKNMFGERVNDPQFQEDLEQIVEWSTIFEDRGIFKAKLQALGWLSEKQIQQLVAKRYKGWGRLSKKLLTGLKNAEGYSILDEMWRSTGNFMQIQSRPEFAALIQQANEKQFEGNDPDNVWENIENILGDAYTSPQNKKAIRQVVKVVQDIEKAVGNPPEKIAIEFTREAAANPQRTQSRLRTLEKLYESAEEVVDAGLTAELAEFKENKHVLSDKYYLYFTQLGRDVYTGDTISLDKLNDYDVDHILPQSFIKDDSLDNRVLTIRAVNNGKSDNVPAKMFGKKMGSFWRYLLDNGMISKRKYNNLITDPDNISKYAQKGFINRQLVETSQVIKLTANILNGIYDKDTEIIEVPAKMNSQMRKMFDLVKVREVNDYHHAFDAYLTIFIGNYLYKCYPKLQPYFVYDNFKKFGNKEDIGHKRFNFLGKIEREKKVVAPETGEILWSNVAPNETIKQIKKVYDYKFMIVSREITTRRAELFNQTVYPKNYHGKLIPIKEDRPTDLYGGYSGNTDAYLAIVALEDKKKGKYFKVVGIPTRVAAKLEKLKQQDSQQYLQALHKVIAPQFTKSTKKGIKKTEFEIVLDKVHYRQLVQDGPVKMMLGSSTYKYNAKQLVLSEKALQVIADDRKFDETQKDDNLIAVYDEILSIVNQSFDLYDINGFRKKLNDNRDQFIDLPAETKYEGRKVVAHGKREMILEILKGLHANAAFGNLKPIGFSTAFGQLQVPNGIILSKNAILIHQSPSGLFERKIKLSDL

SEQ ID NO: 13 - DNA, 36 bp, Artificial Sequence
  Description of Artificial Sequence Synthetic oligonucleotide
ctctagcaggcctggcaaatttctactgttgtagat

SEQ ID NO: 14 - DNA, 35 bp, Artificial Sequence
  Description of Artificial Sequence Synthetic oligonucleotide
gttaagttatatagaataatttctactgttgtaga

SEQ ID NO: 15 - DNA, 29 bp, Artificial Sequence
  Description of Artificial Sequence Synthetic oligonucleotide
actacattttttaagacctaattttgagt

SEQ ID NO: 16 - DNA, 35 bp, Artificial Sequence
  Description of Artificial Sequence Synthetic oligonucleotide
ctcaaaactcattcgaatctctactctttgtagat

SEQ ID NO: 17 - DNA, 36 bp, Artificial Sequence
  Description of Artificial Sequence Synthetic oligonucleotide
gtctaaaactcattcagaatttctactagtgtagat

SEQ ID NO: 18 - DNA, 32 bp, Artificial Sequence
  Description of Artificial Sequence Synthetic oligonucleotide
gtctaggtactctctttaatttctactattgt

SEQ ID NO: 19 - DNA, 33 bp, Artificial Sequence
  Description of Artificial Sequence Synthetic oligonucleotide
gtttaaaaccactttaaaatttctactattgta

SEQ ID NO: 20 - DNA, 24 bp, Artificial Sequence
  Description of Artificial Sequence Synthetic oligonucleotide
ataataatttctacttttgtagat

SEQ ID NO: 21 - DNA, 36 bp, Artificial Sequence
  Description of Artificial Sequence Synthetic oligonucleotide
atctacaatagtagaaatttttaaaaacgatttgac

SEQ ID NO: 22 - DNA, 36 bp, Artificial Sequence; identical to SEQ ID NO: 21
  Description of Artificial Sequence Synthetic oligonucleotide

SEQ ID NO: 23 - DNA, 35 bp, Artificial Sequence
  Description of Artificial Sequence Synthetic oligonucleotide
gtctaacgaccttttaaatttctactgtttgtaga

SEQ ID NO: 24 - DNA, 36 bp, Artificial Sequence
  Description of Artificial Sequence Synthetic oligonucleotide
gtttgagagatatgtaaattcaaaggataatcaaac

SEQ ID NO: 25 - DNA, 37 bp, Artificial Sequence
  Description of Artificial Sequence Synthetic oligonucleotide
ggttttagagttgtgttattttgaacagatacaaaac

SEQ ID NO: 26 - DNA, 36 bp, Artificial Sequence
  Description of Artificial Sequence Synthetic oligonucleotide
gcttgtgtaccatacatttttacatcattctcaaac

SEQ ID NO: 27 - DNA, 36 bp, Artificial Sequence
  Description of Artificial Sequence Synthetic oligonucleotide
gtttgagaatgatgtaaaaatgtatggtactcaagc

SEQ ID NO: 28 - DNA, 36 bp, Artificial Sequence
  Description of Artificial Sequence Synthetic oligonucleotide
gctttagatgtatgtcagattaatggggtttattcc

SEQ ID NO: 29 - DNA, 38 bp, Artificial Sequence
  Description of Artificial Sequence Synthetic oligonucleotide
gtttcagaaggatgttaaatcaataaggttaagatctt

SEQ ID NO: 30 - protein, 1307 aa, Acidamonococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 31 - protein, 1368 aa, Streptococcus pyogenes [sequence omitted]

SEQ ID NO: 32 - DNA, 36 bp, Artificial Sequence
  Description of Artificial Sequence Synthetic oligonucleotide
gttttagaagagtatcaaatcaatgagtagttcaac

SEQ ID NO: 33 - DNA, 36 bp, Artificial Sequence
  Description of Artificial Sequence Synthetic oligonucleotide
gtttgactaccatatgaaattacactactctcaaac

SEQ ID NO: 34 - protein, 7 aa, Simian virus 40
PKKKRKV

SEQ ID NO: 35 - protein, 16 aa, Unknown
  Description of Unknown Nucleoplasmin bipartite NLS sequence
KRPAATKKAGQAKKKK

SEQ ID NO: 36 - protein, 9 aa, Unknown
  Description of Unknown C-myc NLS sequence
PAAKRVKLD

SEQ ID NO: 37 - protein, 11 aa, Unknown
  Description of Unknown C-myc NLS sequence
RQRRNELKRSP

SEQ ID NO: 38 - protein, 38 aa, Homo sapiens
NQSSNFGPMKGGNFGGRSSGPYGGGGQYFAKPRNQGGY

SEQ ID NO: 39 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 40 - protein, 8 aa, Unknown
  Description of Unknown Myoma T protein sequence
VSRKRPRP

SEQ ID NO: 41 - protein, 8 aa, Unknown
  Description of Unknown Myoma T protein sequence
PPKKARED

SEQ ID NO: 42 - protein, 8 aa, Homo sapiens
PQPKKKPL

SEQ ID NO: 43 - protein, 12 aa, Mus musculus
SALIKKKKKMAP

SEQ ID NO: 44 - protein, 5 aa, Influenza virus
DRLRR

SEQ ID NO: 45 - protein, 7 aa, Influenza virus
PKQKKRK

SEQ ID NO: 46 - protein, 10 aa, Hepatitis delta virus
RKLKKKIKKL

SEQ ID NO: 47 - protein, 10 aa, Mus musculus
REKKKFLKRR

SEQ ID NO: 48 - protein, 20 aa, Homo sapiens
KRKGDEVDGVDEVAKKKSKK

SEQ ID NO: 49 - protein, 17 aa, Homo sapiens
RKCLQAGMNLEARKTKK

SEQ ID NO: 50 - protein, 1334 aa, Succinivibrio dextrinosolvens
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 51 - protein, 1211 aa, Candidatus Methanoplasma termitum
MEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 52 - protein, 1235 aa, Candidatus Methanomethylophilus alvus
MHTGGLLSMDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 53 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRHYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 54 - protein, 1318 aa, Flavobacterium branchiophilum
MTNKFTNQYSLSKTLRFELIPQGKTLEFIQEKGLLSQDKQRAESYQEMKKTIDKFHKYFIDLALSNAKLTHLETYLELYNKSAETKKEQKFKDDLKKVQDNLRKEIVKSFSDGDAKSIFAILDKKELITVELEKWFENNEQKDIYFDEKFKTFTTYFTGFHQNRKNMYSVEPNSTAIAYRLIHENLPKFLENAKAFEKIKQVESLQVNFRELMGEFGDEGLIFVNELEEMFQINYYNDVLSQNGITIYNSIISGFTKNDIKYKGLNEYINNYNQTKDKKDRLPKLKQLYKQILSDRISLSFLPDAFTDGKQVLKAIFDFYKINLLSYTIEGQEESQNLLLLIRQTIENLSSFDTQKIYLKNDTHLTTISQQVFGDFSVFSTALNYWYETKVNPKFETEYSKANEKKREILDKAKAVFTKQDYFSIAFLQEVLSEYILTLDHTSDIVKKHSSNCIADYFKNHFVAKKENETDKTFDFIANITAKYQCIQGILENADQYEDELKQDQKLIDNLKFFLDAILELLHFIKPLHLKSESITEKDTAFYDVFENYYEALSLLTPLYNMVRNYVTQKPYSTEKIKLNFENAQLLNGWDANKEGDYLTTILKKDGNYFLAIMDKKHNKAFQKFPEGKENYEKMVYKLLPGVNKMLPKVFFSNKNIAYFNPSKELLENYKKETHKKGDTFNLEHCHTLIDFFKDSLNKHEDWKYFDFQFSETKSYQDLSGFYREVEHQGYKINFKNIDSEYIDGLVNEGKLFLFQIYSKDFSPFSKGKPNMHTLYWKALFEEQNLQNVIYKLNGQAEIFFRKASIKPKNIILHKKKIKIAKKHFIDKKTKTSEIVPVQTIKNLNMYYQGKISEKELTQDDLRYIDNFSIFNEKNKTIDIIKDKRFTVDKFQFHVPITMNFKATGGSYINQTVLEYLQNNPEVKIIGLDRGERHLVYLTLIDQQGNILKQESLNTITDSKISTPYHKLLDNKENERDLARKNWGTVENIKELKEGYISQVVHKIATLMLEENAIVVMEDLNFGFKRGRFKVEKQIYQKLEKMLIDKLNYLVLKDKQPQELGGLYNALQLTNKFESFQKMGKQSGFLFYVPAWNTSKIDPTTGFVNYFYTKYENVDKAKAFFEKFEAIRFNAEKKYFEFEVKKYSDFNPKAEGTQQAWTICTYGERIETKRQKDQNNKFVSTPINLTEKIEDFLGKNQIVYGDGNCIKSQIASKDDKAFFETLLYWFKMTLQMRNSETRTDIDYLISPVMNDNGTFYNSRDYEKLENPTLPKDADANGAYHIAKKGLMLLNKIDQADLTKKVDLSISNRDWLQFVQKNK

SEQ ID NO: 55 - protein, 1230 aa, Lachnospiraceae bacterium COE1
MHENNGKIADNFIGIYPVSKTLRFELKPVGKTQEYIEKHGILDEDLKRAGDYKSVKKIIDAYHKYFIDEALNGIQLDGLKNYYELYEKKRDNNEEKEFQKIQMSLRKQIVKRFSEHPQYKYLFKKELIKNVLPEFTKDNAEEQTLVKSFQEFTTYFEGFHQNRKNMYSDEEKSTAIAYRVVHQNLPKYIDNMRIFSMILNTDIRSDLTELFNNLKTKMDITIVEEYFAIDGFNKVVNQKGIDVYNTILGAFSTDDNTKIKGLNEYINLYNQKNKAKLPKLKPLFKQILSDRDKISFIPEQFDSDTEVLEAVDMFYNRLLQFVIENEGQITISKLLTNFSAYDLNKIYVKNDTTISAISNDLFDDWSYISKAVRENYDSENVDKNKRAAAYEEKKEKALSKIKMYSIEELNFFVKKYSCNECHIEGYFERRILEILDKMRYAYESCKILHDKGLINNISLCQDRQAISELKDFLDSIKEVQWLLKPLMIGQEQADKEEAFYTELLRIWEELEPITLLYNKVRNYVTKKPYTLEKVKLNFYKSTLLDGWDKNKEKDNLGIILLKDGQYYLGIMNRRNNKIADDAPLAKTDNVYRKMEYKLLTKVSANLPRIFLKDKYNPSEEMLEKYEKGTHLKGENFCIDDCRELIDFFKKGIKQYEDWGQFDFKFSDTESYDDISAFYKEVEHQGYKITFRDIDETYIDSLVNEGKLYLFQIYNKDFSPYSKGTKNLHTLYWEMLFSQQNLQNIVYKLNGNAEIFYRKASINQKDVVVHKADLPIKNKDPQNSKKESMFDYDIIKDKRFTCDKYQFHVPITMNFKALGENHFNRKVNRLIHDAENMHIIGIDRGERNLIYLCMIDMKGNIVKQISLNEIISYDKNKLEHKRNYHQLLKTREDENKSARQSWQTIHTIKELKEGYLSQVIHVITDLMVEYNAIVVLEDLNFGFKQGRQKFERQVYQKFEKMLIDKLNYLVDKSKGMDEDGGLLHAYQLTDEFKSFKQLGKQSGFLYYIPAWNTSKLDPTTGFVNLFYTKYESVEKSKEFINNFTSILYNQEREYFEFLFDYSAFTSKAEGSRLKWTVCSKGERVETYRNPKKNNEWDTQKIDLTFELKKLFNDYSISLLDGDLREQMGKIDKADFYKKFMKLFALIVQMRNSDEREDKLISPVLNKYGAFFETGKNERMPLDADANGAYNIARKGLWIIEKIKNTDVEQLDKVKLTISNKEWLQYAQEHIL

SEQ ID NO: 56 - protein, 1264 aa, Prevotella brevis
MKQFTNLYQLSKTLRFELKPIGKTLEHINANGFIDNDAHRAESYKKVKKLIDDYHKDYIENVLNNFKLNGEYLQAYFDLYSQDTKDKQFKDIQDKLRKSIASALKGDDRYKTIDKKELIRQDMKTFLKKDTDKALLDEFYEFTTYFTGYHENRKNMYSDEAKSTAIAYRLIHDNLPKFIDNIAVFKKIANTSVADNFSTIYKNFEEYLNVNSIDEIFSLDYYNIVLTQTQIEVYNSIIGGRTLEDDTKIQGINEFVNLYNQQLANKKDRLPKLKPLFKQILSDRVQLSWLQEEFNTGADVLNAVKEYCTSYFDNVEESVKVLLTGISDYDLSKIYITNDLALTDVSQRMFGEWSIIPNAIEQRLRSDNPKKTNEKEEKYSDRISKLKKLPKSYSLGYINECISELNGIDIADYYATLGAINTESKQEPSIPTSIQVHYNALKPILDTDYPREKNLSQDKLTVMQLKDLLDDFKALQHFIKPLLGNGDEAEKDEKFYGELMQLWEVIDSITPLYNKVRNYCTRKPFSTEKIKVNFENAQLLDGWDENKESTNASIILRKNGMYYLGIMKKEYRNILTKPMPSDGDCYDKVVYKFFKDITTMVPKCTTQMKSVKEHFSNSNDDYTLFEKDKFIAPVVITKEIFDLNNVLYNGVKKFQIGYLNNTGDSFGYNHAVEIWKSFCLKFLKAYKSTSIYDFSSIEKNIGCYNDLNSFYGAVNLLLYNLTYRKVSVDYIHQLVDEDKMYLFMIYNKDFSTYSKGTPNMHTLYWKMLFDESNLNDVVYKLNGQAEVFYRKKSITYQHPTHPANKPIDNKNVNNPKKQSNFEYDLIKDKRYTVDKFMFHVPITLNFKGMGNGDINMQVREYIKTTDDLHFIGIDRGERHLLYICVINGKGEIVEQYSLNEIVNNYKGTEYKTDYHTLLSERDKKRKEERSSWQTIEGIKELKSGYLSQVIHKITQLMIKYNAIVLLEDLNMGFKRGRQKVESSVYQQFEKALIDKLNYLVDKNKDANEIGGLLHAYQLTNDPKLPNKNSKQSGFLFYVPAWNTSKIDPVTGFVNLLDTRYENVAKAQAFFKKFDSIRYNKEYDRFEFKFDYSNFTAKAEDTRTQWTLCTYGTRIETFRNAEKNSNWDSREIDLTTEWKTLFTQHNIPLNANLKEAILLQANKNFYTDILHLMKLTLQMRNSVTGTDIDYMVSPVANECGEFFDSRKVKEGLPVNADANGAYNIARKGLWLAQQIKNANDLSDVKLAITNKEWLQFAQKKQYLKD

SEQ ID NO: 57 - protein, 1274 aa, Smithella sp.
MKQFTNQFSLSKTLRFELIPQGKTKEFIEINGLIEKDNERAVSYKKVKKIIDEYHKYFIEMVLCDFKLHGLETYETIFNKKEKDDTDKKEFDNIRNSLRKQIADAFAKNPNDEIKERFKNLFAKELIKQDLLNFVDDEQKELVNEFKDFTTYFTGFHQNRRNMYVADEKATAIAYRLVNENLPKFIDNLKIYEKIKKDAPELISDLNKTLVEMEEIVQGKTLDEIFSLSFFNQTLTQTGIELYNIVIGGRTADEGKTKIKGLNEYINTDYNQKQTDKKKKQAKFKQLYKQILSDRHSVSFVAETFETDAQLLENIEQFYSSVLCNYEDDGHTTNIFEAIKNLIIGLKTFDLSKIYLRNDTSLTDISQKLFGDWSIISSALNDYYEKQNPISSKEKQEKYDERKAKWLKQDFNIETIQTALNECDSEIIKEKNNKNIVSEYFAKLGLDKDNKIDLLQKIHHNYVVIKDLLNEPYPENIKLGNQKEQVSQIKDFLDSILNLIHFLKPLSLKDKDKEKDELFYSLFTALFEHLSQTISIYNKVRNYLTQKAYSTEKIKLNFENSTLLNGWDVNKEPVNTSVIFRKNGLFYLGIMSKSNNRIFERNVPVCKNEETAFEKMNYKLLPGANKMLPKVFLSAKGIESFQPSAEIQSKYQKETHKKGDAFVRKDMENLIDFFKQSIAKHTDWKHFNHQFSKTETYNDLSEFYKEVEKQGYKLTFTKLDETYINQLVDEGKLYLFQIYNKDFSPFSKGKPNMHTLYWKMLFDEQNLQNVVYKLNGEAEVFFRQSSIKQTDRIIHKANQAIDNKNPLNNKKQSSFNYDLIKDKRFTLDKFQFHVPITLNFKAEGNEYLNTKVNEYLKSNSDVKIIGLDRGERHLIYLTLINQKGELLKQQSLNVIATSQEHETDYKNLLVNKENERANARQDWKTIETIKELKEGYLSQVVHQIATMMVDENAIVVMEDLNAGFMRGRQKVERQVYQKLEKMLIEKLNYLVFKNNDVNETAGVLNALQLTNKFESFEKMGKQSGFLFYVPAWNTSKIDPATGFVDFLKPKYESVEKAKLFFEKFESIKFNADKNYFEFEFDYKKFTEKAEGSQTKWTVCTHSDVRYRYNPQTKASDEVNVTNELKLIFDKFKIEYKNGKNLKTELLLQDDKQLFSKLLHYLALTLMLRQSKSGTDIDFILSPVAKNGVFYDSRNAMPNLPKDADANGAFHIALKGLWCVQQIKKADDLKKIKLAISNKEWLSFVQNLKEVMTEAKLFQKALLLTENMKKHQLEL

SEQ ID NO: 58 - protein, 1217 aa, Moraxella bovoculi
MYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 59 - protein, 1259 aa, Synergistes jonesii
MANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 60 - protein, 1262 aa, Bacteroidetes oral taxon 274
MRKFNEFVGLYPISKTLRFELKPIGKTLEHIQRNKLLEHDAVRADDYVKVKKIIDKYHKCLIDEALSGFTFDTEADGRSNNSLSEYYLYYNLKKRNEQEQKTFKTIQNNLRKQIVNKLTQSEKYKRIDKKELITTDLPDFLTNESEKELVEKFKNFTTYFTEFHKNRKNMYSKEEKSTAIAFRLINENLPKFVDNIAAFEKVVSSPLAEKINALYEDFKEYLNVEEISRVFRLDYYDELLTQKQIDLYNAIVGGRTEEDNKIQIKGLNQYINEYNQQQTDRSNRLPKLKPLYKQILSDRESVSWLPPKFDSDKNLLIKIKECYDALSEKEKVFDKLESILKSLSTYDLSKIYISNDSQLSYISQKMFGRWDIISKAIREDCAKRNPQKSRESLEKFAERIDKKLKTIDSISIGDVDECLAQLGETYVKRVEDYFVAMGESEIDDEQTDTTSFKKNIEGAYESVKELLNNADNITDNNLMQDKGNVEKIKTLLDAIKDLQRFIKPLLGKGDEADKDGVFYGEFTSLWTKLDQVTPLYNMVRNYLTSKPYSTKKIKLNFENSTLMDGWDLNKEPDNTTVIFCKDGLYYLGIMGKKYNRVFVDREDLPHDGECYDKMEYKLLPGANKMLPKVFFSETGIQRFLPSEELLGKYERGTHKKGAGFDLGDCRALIDFFKKSIERHDDWKKFDFKFSDTSTYQDISEFYREVEQQGYKMSFRKVSVDYIKSLVEEGKLYLFQIYNKDFSAHSKGTPNMHTLYWKMLFDEENLKDVVYKLNGEAEVFFRKSSITVQSPTHPANSPIKNKNKDNQKKESKFEYDLIKDRRYTVDKFLFHVPITMNFKSVGGSNINQLVKRHIRSATDLHIIGIDRGERHLLYLTVIDSRGNIKEQFSLNEIVNEYNGNTYRTDYHELLDTREGERTEARRNWQTIQNIRELKEGYLSQVIHKISELAIKYNAVIVLEDLNFGFMRSRQKVEKQVYQKFEKMLIDKLNYLVDKKKPVAETGGLLRAYQLTGEFESFKTLGKQSGILFYVPAWNTSKIDPVTGFVNLFDTHYENIEKAKVFFDKFKSIRYNSDKDWFEFVVDDYTRFSPKAEGTRRDWTICTQGKRIQICRNHQRNNEWEGQEIDLTKAFKEHFEAYGVDISKDLREQINTQNKKEFFEELLRLLRLTLQMRNSMPSSDIDYLISPVANDTGCFFDSRKQAELKENAVLPMNADANGAYNIARKGLLAIRKMKQEENDSAKISLAISNKEWLKFAQTKPYLED

SEQ ID NO: 61 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 62 - protein, 1263 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 63 - protein, 1368 aa, Lactobacillus curvatus
MSRPYNISLDIGTSSIGWSVVDDQSKLVSVRGKYGYGVRLYDEGQTAAERRSFRTTRRRLKRRKWRLGLLREIFEPYITPVDDTFFLRQKQSNLSPKDQRKLYPQTSLFNDRTDRAFYDDYPTIYHLRYKLMTEKRQFDIREIYLAMHHIVKYRGHFLNEAPVSSFKSSEINLVAHFDRLNTIFADLFSESGFQLETDKLAEVKALLLDNQQSASNRQRQALSLIYTPSTNKAVEKQNKAIATELLKAILGLKAKFNVLTGIEAEDVKAWTLTFNAENFDEEMVKLESSLDDNAHQIIESLQELYSGVLLAGIVPENQSLSQAMITKYDDHQKHLKMLKAVREALAPEDRQRLKQAYDQYVDGQENTKAYSKEDFYGDITKALKNNPDHPIVSEIKKLIELDQFMPKQRTKDNGAIPHQLHQQELDRIIENQQQYYPWLAELNPNSKRQTVAKYKLDELVAFRVPYYVGPLITAEQQQQSSDAKFAWMIRKAEGRITPWNFDDKVDRQASANEFIKRMTTTDTYLLAEDVLPKQSLIYQRFEVLNELNGLKIDDQPITTELKQAIFTDLFMQKISVTVKNIQDYLVSEKRYASRPAITGLSDENKFNSRLSTYHDLKAIVGDAVEDVDKQVDLEKCVEWSTIFEDGKIYSAKLNEIDWLTDQQRVQLAAKRYRGWGRLSAKLLTQIVNANGQRIMDLLWDTTDNFMRIVHSEDFDKLITEANQMMLAENDVQDVINDLYTSPQNKKALRQILLVVNDIQKAMKGQAPERILIEFAREDEVNRRLSVQRKRQVEQVYQNISNELLNNTEIRNELKDLSNSALSNTRLFLYFMQGGRDMYTGDSLNIDRLSTYDIDHILPQSFIKDNSLDNRVLVSQKMNRSKADQVPTDFTSVELGKKMQLQWEQMLRAGLITKKKYDNLTLNPDHISKYAMKGFINRQLVETRQVIKLATNLLMEQYGEDNIELITVKSGLTHQMRTEFDFPKNRNLNNHHHAFDAYLTAFVGLYLLKRYPKLKPYFVYGEYQKASQQDKWRNFNFLNGLKKDELVDENTEAVIWDKESGLAYLNKIYQFKKILVTREVHENSGALFNQTLYAAKDDKASGQGSKQLIPAKQNRPTALYGGYSGKTVAYMCIVRIKNKKGDLYKVCGVETSWLAQLKQLTDEDSQKAFLKQKISPQFTKVKKQKGTIVKVVEDFEVIAPHILINQRFFDNGQELTLGSATYKHNEQELILDKTAVKLLNGALPLTQSEELAEQVYDEILDQVMHYFPLYDTNQFRAKLSAGKAAFMKLPWKSQWDGNKMVQVGQQVILDRVLIGLHANAAVSDLGILKLTTPFGKLQKSSGIYLSPDTQIIYQSPTGLFERRVALRDL

SEQ ID NO: 64 - protein, 1347 aa, Lactobacillus versmoldensis
MTKREEPYNVGLDIGSSSVGWAVVDNNYHLLNIKKNNLWGARLFKEAETAQVTRGHRSMRRRYRRRRNRLNWLDELFADELAKIDPSFLLRMKNSWVSKKESTRKRDPYNLFIDEKYNDVDYFNQYPTIFHLRKELITEDKKVDIRLIYLAIHNIIKYRGNFTLENQNFDISQLSSNFSQQISDFFALFSDFGVIMPEDFDPDKISDILLNPNLSPSGKVSEAIATISPKTNVKAKIKIILLLLVGNNGDLKKLFDLETTEKIAVKLSSRHIDSELPIILSELNEQQENIITIANSIFGSIILKDFLGDETSISAAKVISFEDHKQDLQKLKTMWRETSNKEAVKAGKKAYEDYIGHEDSETFYKKIKKFLEKAQPVDLANKALAEIELENYLPKQRNRNNTVIPYQLNENELIAILDHQEKYYPFLKENRDKILSLLTFRIPYYVGPLQDSNNNRFSWMTRKASGAIRPWNFSEKVNVEQSSNDFIKRMRSTDTYLIGEPVLPKKSLIYQCYEVLSELNNTRVKDGSSNPKRLDVTIKQRIYNEIFKNQKSVSVKVLQNWLIKESYFKSPEISGLADKKKYLSSLSTYIDFKKIFGQRFVDDPVNSPQLEELAEWLTLFEDKKILLIKLQNSKYSYDQATINKLSTMRYQGTGKLSKKLLVDLKTTKKSIGKSGAESLSILDLMWSTKDNFMQIIHDADYTFEQQIKEFNYDTEDELTPLEKVANLHGSPALKRGLNQSIKVVADIVKFMGHDPEKIFIEFTRSDDFSKLTISRYRRIKKQYLEIAKAIKKIPAEFKDIKEYQTQLEENKGKLASERLMLYFLQCGHSLYSNKPIDLNMINSSKYHVDHILPQSYIKDDSLENKALVLASENENKIDNMLISHDIIATNLPRWQALKDQNLMGSKKFADLTRTTVTENQKKGFIQRQLVQTSQIVKNITLILNDLYKNTSCIETRATLSSEFRKAFSNFDETTYHYQFPEFVKNRDVNDFHHAQDAFLACVIGEYQLKKYPKDNLRLVYDQYSKFLDSLKKDTRKKNGRMPRYTQNGFIIGSMFNGKTYVDDNGEIIWDQKIKESIRKTFNYHQFNVVRQTIEQHGKLFNDTIQPHSDRYKLIPLKTNRDPAIYGGYNNDNNAYSVVLDVDGKKKINGIPIRIANQLKSDELDLSSWLENNIKHKKPMTILIDKVPKYQRIINEETGDLLITSANEVINNVQLFLPSMYTALISLLDSTKTEMYSKLLSNYEANILIDIYDYLLTKLKNNYPLYRKEWAKLAEHRDDFIESDLVTQASTLQQLIKFMHADPSNVNLKFGNFKGNRFGRKNGNIKLSKTDFIYESPTGLFKSIKHID

SEQ ID NO: 65 - protein, 1365 aa, Filifactor alocis
MTKEYYLGLDVGTNSVGWAVTDSQYNLCKFKKKDMWGIRLFESANTAKDRRLQRGNRRRLERKKQRIDLLQEIFSPEICKIDPTFFIRLNESRLHLEDKSNDFKYPLFIEKDYSDIEYYKEFPTIFHLRKHLIESEEKQDIRLIYLALHNIIKTRGHFLIDGDLQSAKQLRPILDTFLLSLQEEQNLSVSLSENQKDEYEEILKNRSIAKSEKVKKLKNLFEISDELEKEEKKAQSAVIENFCKFIVGNKGDVCKFLRVSKEELEIDSFSFSEGKYEDDIVKNLEEKVPEKVYLFEQMKAMYDWNILVDILETEEYISFAKVKQYEKHKTNLRLLRDIILKYCTKDEYNRMFNDEKEAGSYTAYVGKLKKNNKKYWIEKKRNPEEFYKSLGKLLDKIEPLKEDLEVLTMMIEECKNHTLLPIQKNKDNGVIPHQVHEVELKKILENAKKYYSFLTETDKDGYSVVQKIESIFRFRIPYYVGPLSTRHQEKGSNVWMVRKPGREDRIYPWNMEEIIDFEKSNENFITRMTNKCTYLIGEDVLPKHSLLYSKYMVLNELNNVKVRGKKLPTSLKQKVFEDLFENKSKVTGKNLLEYLQIQDKDIQIDDLSGFDKDFKTSLKSYLDFKKQIFGEEIEKESIQNMIEDIIKWITIYGNDKEMLKRVIRANYSNQLTEEQMKKITGFQYSGWGNFSKMFLKGISGSDVSTGETFDIITAMWETDNNLMQILSKKFTFMDNVEDFNSGKVGKIDKITYDSTVKEMFLSPENKRAVWQTIQVAEEIKKVMGCEPKKIFIEMARGGEKVKKRTKSRKAQLLELYAACEEDCRELIKEIEDRDERDFNSMKLFLYYTQFGKCMYSGDDIDINELIRGNSKWDRDHIYPQSKIKDDSIDNLVLVNKTYNAKKSNELLSEDIQKKMHSFWLSLLNKKLITKSKYDRLTRKGDFTDEELSGFIARQLVETRQSTKAIADIFKQIYSSEVVYVKSSLVSDFRKKPLNYLKSRRVNDYHHAKDAYLNIVVGNVYNKKFTSNPIQWMKKNRDTNYSLNKVFEHDVVINGEVIWEKCTYHEDTNTYDGGTLDRIRKIVERDNILYTEYAYCEKGELFNATIQNKNGNSTVSLKKGLDVKKYGGYFSANTSYFSLIEFEDKKGDRARHIIGVPIYIANMLEHSPSAFLEYCEQKGYQNVRILVEKIKKNSLLIINGYPLRIRGENEVDTSFKRAIQLKLDQKNYELVRNIEKFLEKYVEKKGNYPIDENRDHITHEKMNQLYEVLLSKMKKFNKKGMADPSDRIEKSKPKFIKLEDLIDKINVINKMLNLLRCDNDTKADLSLIELPKNAGSFVVKKNTIGKSKIILVNQSVTGLYENRREL

SEQ ID NO: 66 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEEYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKIYFRSGTSLTDVSRKVFGEWSIINRALDNYYATTYPIKPREKSEKYEERKEKWLKQDFNVSLIQTAIDEYDNETVKGKNSGKVIVDYFAKFCDDKETDLIQKVNEGYIAVKDLLNTPYPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKENLYYLGIMDKRHNRIFRNVPKADKKDSCYEKMVYKLLPGANKMLPKVFFSQSRIQEFTPSAKLLENYENETHKKGDNFNLNHCHQLIDFFKDSINKHEDWKNFDFRFSATSTYADLSGFYHEVEHQGYKISFQSIADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHVPITMNFKAEGIFNMNQRVNQFLKANPDINIIGIDRGERHLLYYTLINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLKQAKDFFEKFDSIRLNSKADYFEFAFDFKNFTGKADGGRTKWTVCTTNEDRYAWNRALNNNRGSQEKYDITAELKSLFDGKVDYKSGKDLKQQIASQELADFFRTLMKYLSVTLSLRHNNGEKGETEQDYILSPVADSMGKFFDSRKAGDDMPKNADANGAYHIALKGLWCLEQISKTDDLKKVKLAISNKEWLEFMQTLKG

SEQ ID NO: 67 - DNA, 2278 bp, Succinivibrio dextrinosolvens
aagcattggccgtaagtgcgattccggaaaggagatatacatgcaccatcatcatcaccattcatcgctcacgaaattcactaacaaatactctaaacagctcaccattaagaatgaactcatcccagttggcaaaacactggagaacatcaaagagaatggtctgatagatggcgacgaacagctgaatgagaattatcagaaggcgaaaattattgtggatgattttctgcgggacttcattaataaagcactgaataatacgcagatcgggaactggcgcgaactggcggatgcccttaataaagaggatgaagataacatcgagaaattgcaggataaaattcggggaatcattgtatccaaatttgaaacgtttgatctgtttagcagctattctattaagaaagatgaaaagattattgacgacgacaatgatgttgaagaagaggaactggatctgggcaagaagaccagctcatttaaatacatatttaaaaaaaacctgtttaagttagtgttgccatcctacctgaaaaccacaaaccaggacaagctgaagattattagctcgtttgataatttttcaacgtacttccgcgggttctttgaaaaccggaaaaacatttttaccaagaaaccgatctccacaagtattgcgtatcgcattgttcatgataacttcccgaaattccttgataacattcgttgttttaatgtgtggcagacggaatgcccgcaactaatcgtgaaagcagataactatctgaaaagcaaaaatgttatagcgaaagataaaagtttggcaaactattttaccgtgggcgcgtatgactatttcctgtctcagaatggtatagatttttacaacaatattataggtggactgccagcgttcgccggccatgagaaaatccaaggtctcaatgaattcatcaatcaagagtgccaaaaagacagcgagctgaaaagtaagctgaaaaaccgtcacgcgttcaaaatggcggtactgttcaaacagatactcagcgatcgtgaaaaaagttttgtaattgatgagttcgagtcggatgctcaagttattgacgccgttaaaaacttttacgccgaacagtgcaaagataacaatgttatttttaacttattaaatcttatcaagaatatcgctttcttaagtgatgacgaactggacggcatattcattgaagggaaatacctgtcgagcgttagtcaaaaactctatagcgattggtcaaaattacgtaacgacattgaggattcggctaactctaaacaaggcaataaagagctggccaagaagatcaaaaccaacaaaggggatgtagaaaaagcgatctcgaaatatgagttctcgctgtcggaactgaactcgattgtacatgataacaccaagttttctgacctccttagttgtacactgcataaggtggcttctgagaaactggtgaaggtcaatgaaggcgactggccgaaacatctcaagaataatgaagagaaacaaaaaatcaaagagccgcttgatgctctgctggagatctataatacacttctgatttttaactgcaaaagcttcaataaaaacggcaacttctatgtcgactatgatcgttgcatcaatgaactgagttcggtcgtgtatctgtataataaaacacgtaactattgcactaaaaaaccctataacacggacaagttcaaactcaattttaacagtccgcagctcggtgaaggcttttccaagtcgaaagaaaatgactgtctgactcttttgtttaaaaaagacgacaactattatgtaggcattatccgcaaaggtgcaaaaatcaattttgatgatacacaagcaatcgccgataacaccgacaattgcatctttaaaatgaattatttcctacttaaagacgcaaaaaaatttatcccgaaatgtagcattcagctgaaagaagtcaaggcccattttaagaaatctgaagatgattacattttgtctgataaagagaaatttgctagcccgctggtcattaaaaagagcacatttttgctggcaactgcacatgtgaaagggaaaaaaggcaatatcaagaaatttcagaaagaatattcgaaagaaaaccccactgagtatcgcaattctttaaacgaatggattgctttttgtaaagagttcttaaaaacttataaagcggctaccatttttgatataaccacattgaaaaaggcagaggaatatgctgatattgtagaattctacaaggat

SEQ ID NO: 68 - DNA, 2062 bp, Candidatus Methanoplasma termitum
aagcattggccgtaagtgcgattccggaaaggagatatacatgcaccatcatcatcaccataacaactacgacgaattcaccaaactgtacccgatccagaaaaccatccgtttcgaactgaaaccgcagggtcgtaccatggaacacctggaaaccttcaacttcttcgaagaagaccgtgaccgtgcggaaaaatacaaaatcctgaaagaagcgatcgacgaataccacaaaaaattcatcgacgaacacctgaccaacatgtctctggactggaactctctgaaacagatctctgaaaaatactacaaatctcgtgaagaaaaagacaaaaaagttttcctgtctgaacagaaacgtatgcgtcaggaaatcgtttctgaattcaaaaaagacgaccgtttcaaagacctgttctctaaaaaactgttctctgaactgctgaaagaagaaatctacaaaaaaggtaaccaccaggaaatcgacgcgctgaaatctttcgacaaattctctggttacttcatcggtctgcacgaaaaccgtaaaaacatgtactctgacggtgacgaaatcaccgcgatctctaaccgtatcgttaacgaaaacttcccgaaattcctggacaacctgcagaaataccaggaagcgcgtaaaaaatacccggaatggatcatcaaagcggaatctgcgctggttgcgcacaacatcaaaatggacgaagttttctctctggaatacttcaacaaagttctgaaccaggaaggtatccagcgttacaacctggcgctgggtggttacgttaccaaatctggtgaaaaaatgatgggtctgaacgacgcgctgaacctggcgcaccagtctgaaaaatcttctaaaggtcgtatccacatgaccccgctgttcaaacagatcctgtctgaaaaagaatctttctcttacatcccggacgttttcaccgaagactctcagctgctgccgtctatcggtggtttcttcgcgcagatcgaaaacgacaaagacggtaacatcttcgaccgtgcgctggaactgatctcttcttacgcggaatacgacaccgaacgtatctacatccgtcaggcggacatcaaccgtgtttctaacgttatcttcggtgaatggggtaccctgggtggtctgatgcgtgaatacaaagcggactctatcaacgacatcaacctggaacgtacctgcaaaaaagttgacaaatggctggactctaaagaattcgcgctgtctgacgttctggaagcgatcaaacgtaccggtaacaacgacgcgttcaacgaatacatctctaaaatgcgtaccgcgcgtgaaaaaatcgacgcggcgcgtaaagaaatgaaattcatctctgaaaaaatctctggtgacgaagaatctatccacatcatcaaaaccctgctggactctgttcagcagttcctgcacttcttcaacctgttcaaagcgcgtcaggacatcccgctggacggtgcgttctacgcggaattcgacgaagttcactctaaactgttcgcgatcgttccgctgtacaacaaagttcgtaactacctgaccaaaaacaacctgaacaccaaaaaaatcaaactgaacttcaaaaacccgaccctggcgaacggttgggaccagaacaaagtttacgactacgcgtctctgatcttcctgcgtgacggtaactactacctgggtatcatcaacccgaaacgtaaaaaaaacatcaaattcgaacagggttctggtaacggtccgttctaccgtaaaatggtttacaaacagatcccgggtccgaacaaaaacctgccgcgtgttttcctgacctctaccaaaggtaaaaaagaatacaaaccgtctaaagaaatcatcgaaggttacgaagcggacaaacacatccgtggtgacaaattcgacctggacttctgccacaaactgatcgacttcttcaaagaatctatcgaaaaacacaaagactggtctaaattcaacttctacttctctccgaccgaatcttacggtgacatctctgaattctacctggac

SEQ ID NO: 69 - DNA, 1917 bp, Thiomicrospira sp.
actaaaacatttgattcagagttttttaatttgtactcgctgcaaaaaacggtacgctttgagttaaaacccgtgggagaaaccgcgtcatttgtggaagactttaaaaacgagggcttgaaacgtgttgtgagcgaagatgaaaggcgagccgtcgattaccagaaagttaaggaaataattgacgattaccatcgggatttcattgaagaaagtttaaattattttccggaacaggtgagtaaagatgctcttgagcaggcgtttcatctttatcagaaactgaaggcagcaaaagttgaggaaagggaaaaagcgctgaaagaatgggaagcgctgcagaaaaagctacgtgaaaaagtggtgaaatgcttctcggactcgaataaagcccgcttctcaaggattgataaaaaggaactgattaaggaagacctgataaattggttggtcgcccagaatcgcgaggatgatatccctacggtcgaaacgtttaacaacttcaccacatattttaccggcttccatgagaatcgtaaaaatatttactccaaagatgatcacgccaccgctattagctttcgccttattcatgaaaatcttccaaagttttttgacaacgtgattagcttcaataagttgaaagagggtttccctgaattaaaatttgataaagtgaaagaggatttagaagtagattatgatctgaagcatgcgtttgaaatagaatatttcgttaacttcgtgacccaagcgggcatagatcagtataattatctgttaggagggaaaaccctggaggacgggacgaaaaaacaagggatgaatgagcaaattaatctgttcaaacaacagcaaacgcgagataaagcgcgtcagattcccaaactgatccccctgttcaaacagattcttagcgaaaggactgaaagccagtcctttattcctaaacaatttgaaagtgatcaggagttgttcgattcactgcagaagttacataataactgccaggataaattcaccgtgctgcaacaagccattctcggtctggcagaggcggatcttaagaaggtcttcatcaaaacctctgatttaaatgccttatctaacaccattttcgggaattacagcgtcttttccgatgcactgaacctgtataaagaaagcctgaaaacgaaaaaagcgcaggaggcttttgagaaactaccggcccattctattcacgacctcattcaatacttggaacagttcaattccagcctggacgcggaaaaacaacagagcaccgacaccgtcctgaactacttcatcaagaccgatgaattatattctcgcttcattaaatccactagcgaggctttcactcaggtgcagcctttgttcgaactggaagccctgtcatctaagcgccgcccaccggaatcggaagatgaaggggcaaaagggcaggaaggcttcgagcagatcaagcgtattaaagcttacctggatacgcttatggaagcggtacactttgcaaagccgttgtatcttgttaagggtcgtaaaatgatcgaagggctcgataaagaccagtccttttatgaagcgtttgaaatggcgtaccaagaacttgaatcgttaatcattcctatctataacaaagcgcggagctatctgtcgcggaaacctttcaaggccgataaattcaagattaattttgacaacaacacgctactgagcggatgggatgcgaacaaggaaactgctaacgcgtccattctgtttaagaaagacgggttatattaccttggaattatgccgaaaggtaagacctttctctttgactactttgtatcgagcgaggattcagagaaactgaaacagcgtcgccagaagaccgccgaagaagctctggcgcaggatggtgaaagttac

SEQ ID NO: 70 - DNA, 2044 bp, Candidatus Methanomethylophilus alvus
aagcattggccgtaagtgcgattccggaaaggagatatacatgcaccatcatcatcaccatcatacaggcggtcttcttagtatggacgcgaaagagttcacaggtcagtatccgttgtcgaaaacattacgattcgaacttcggcccatcggccgcacgtgggataacctggaggcctcaggctacttagcggaagaccgccatcgtgccgaatgttatcctcgtgcgaaagagttattggatgacaaccatcgtgccttcctgaatcgtgtgttgccacaaatcgatatggattggcacccgattgcggaggccttttgtaaggtacataaaaaccctggtaataaagaacttgcccaggattacaaccttcagttgtcaaagcgccgtaaggagatcagcgcatatcttcaggatgcagatggctataaaggcctgttcgcgaagcccgccttagacgaagctatgaaaattgcgaaagaaaacgggaacgaaagtgatattgaggttctcgaagcgtttaacggttttagcgtatacttcaccggttatcatgagtcacgcgagaacatttatagcgatgaggatatggtgagcgtagcctaccgaattactgaggataatttcccgcgctttgtctcaaacgctttgatctttgataaattaaacgaaagccatccggatattatctctgaagtatcgggcaatcttggagttgatgacattggtaagtactttgacgtgtcgaactataacaattttctttcccaggccggtatagatgactacaatcacattattggcggccatacaaccgaagacggactgatacaagcgtttaatgtcgtattgaacttacgtcaccaaaaagaccctggctttgaaaaaattcagttcaaacagctctacaaacaaatcctgagcgtgcgtaccagcaaaagctacatcccgaaacagtttgacaactctaaggagatggttgactgcatttgcgattatgtcagcaaaatagagaaatccgaaacagtagaacgggccctgaaactagtccgtaatatcagttctttcgacttgcgcgggatctttgtcaataaaaagaacttgcgcatactgagcaacaaactgataggagattgggacgcgatcgaaaccgcattgatgcatagttcttcatcagaaaacgataagaaaagcgtatatgatagcgcggaggcttttacgttggatgacatcttttcaagcgtgaaaaaattttctgatgcctctgccgaagatattggcaacagggcggaagacatctgtagagtgataagtgagacggccccttttatcaacgatctgcgagcggtggacctggatagcctgaacgacgatggttatgaagcggccgtctcaaaaattcgggagtcgctggagccttatatggatcttttccatgaactggaaattttctcggttggcgatgagttcccaaaatgcgcagcattttacagcgaactggaggaagtcagcgaacagctgatcgaaattattccgttattcaacaaggcgcgttcgttctgcacccggaaacgctatagcaccgataagattaaagtgaacttaaaattcccgaccttggcggacgggtgggacctgaacaaagagagagacaacaaagccgcgattctgcggaaagacggtaagtattatctggcaattctggatatgaagaaagatctgtcaagcattaggaccagcgacgaagatgaatccagcttcgaaaagatggagtataaactgttaccgagtccagtaaaaatgctgccaaagatattcgtaaaatcgaaagccgctaaggaaaaatatggcctgacagatcgtatgcttgaatgctacgataaaggtatgcataagtcgggtagtgcgtttgatcttggcttttgccatgaactcattgattattacaagcgttgtatcgcggagtacccaggctgggatgtgttcgatttcaagtttcgcgaaacttccgattatgggtccatgaaagagttcaatgaagat

SEQ ID NO: 71 - DNA, 1866 bp, Porphyromonas crevioricanis
gatagtttgaaagatttcaccaatctgtaccctgtcagtaagacattgagatttgaattaaagcccgttggaaagactttagaaaatatcgagaaagcaggtattttgaaagaggatgagcatcgtgcagaaagttatcggagggtgaagaaaataattgatacttatcataaggtatttatcgattcttctcttgaaaatatggctaaaatgggtattgagaatgaaataaaagcaatgctccaaagtttctgcgaattgtataaaaaagatcatcgcactgagggtgaagacaaggcattagataaaattcgagcagtacttcgtggcctgattgttggggctttcactggtgtttgcggaagacgggaaaatacagtccaaaacgagaagtacgagagtttgttcaaagaaaagttgataaaagaaattttacctgattttgtgctctctactgaggctgaaagcttgcctttctctgttgaagaagctacgaggtcactgaaggagtttgatagctttacatcctactttgctggtttttacgagaatagaaagaatatatactcgacgaaacctcaatccactgccattgcttatcgtcttattcatgagaacttgccgaagttcattgataatattcttgtttttcagaagatcaaagagcctatagccaaagagctggaacatattcgtgcggacttttctgccggggggtacataaaaaaggatgagagattggaggatattttttcgttgaactattatatccacgtgttatctcaggctgggatcgaaaaatataacgcattgattgggaagattgtgacagaaggagatggagagatgaaagggctcaatgaacacatcaacctttacaaccaacaaagaggcagagaggatcggctccctctttttaggcctctttataaacagatattgagtgacagagagcaattatcatacttgcctgagagttttgaaaaagatgaggagctcctcagggctctaaaagagttctatgatcatatcgcagaagacattctcggacgtactcaacagttgatgacttctatttcagaatatgatttatctcggatatacgtaaggaacgatagccaattgactgatatatcaaaaaaaatgttgggagattggaatgctatctacatggctagagaacgagcatatgaccacgagcaggctcccaaaagaatcacggcgaaatacgagagggacaggattaaagctcttaaaggagaagagagtataagtctggcaaatcttaatagttgtattgcctttctggacaatgttagagattgccgtgtagatacttatctttccacactgggccagaaggaaggaccacatggtctatctaatctcgttgagaacgtttttgcctcataccatgaagcagagcaattgttgagctttccataccccgaagagaataatctgattcaggacaaggacaatgtggtgttaattaagaatcttctcgacaatatcagtgatctgcagaggttcttgaaacctctttggggtatgggagacgaacccgataaagatgaaagattttatggagagtataattatatccgaggagctctagatcaggtgatccctctgtacaataaggtaaggaactacctcactcggaagccttattcgaccagaaaagtaaaactcaattttgggaattctcaattgcttagtggttgggatagaaataaggaaaaggataatagctgtgtgattttgcgtaaggggcagaacttctatttggctattatgaacaataggcacaaaagaagtttcgaaaacaaggtgttgcccgagtataaggagggagaaccttac

SEQ ID NO: 72 - DNA, 2096 bp, Eubacterium rectale
aagcattggccgtaagtgcgattccggaaaggagatatacatgaacaacggcacaaataattttcagaacttcatcgggatctcaagtttgcagaaaacgctgcgcaatgctctgatccccacggaaaccacgcaacagttcatcgtcaagaacggaataattaaagaagatgagttacgtggcgagaaccgccagattctgaaagatatcatggatgactactaccgcggattcatctctgagactctgagttctattgatgacatagattggactagcctgttcgaaaaaatggaaattcagctgaaaaatggtgataataaagataccttaattaaggaacagacagagtatcggaaagcaatccataaaaaatttgcgaacgacgatcggtttaagaacatgtttagcgccaaactgattagtgacatattacctgaatttgtcatccacaacaataattattcggcatcagagaaagaggaaaaaacccaggtgataaaattgttttcgcgctttgcgactagctttaaagattacttcaagaaccgtgcaaattgcttttcagcggacgatatttcatcaagcagctgccatcgcatcgtcaacgacaatgcagagatattcttttcaaatgcgctggtctaccgccggatcgtaaaatcgctgagcaatgacgatatcaacaaaatttcgggcgatatgaaagattcattaaaagaaatgagtctggaagaaatatattcttacgagaagtatggggaatttattacccaggaaggcattagcttctataatgatatctgtgggaaagtgaattcttttatgaacctgtattgtcagaaaaataaagaaaacaaaaatttatacaaacttcagaaacttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacatta

SEQ ID NO: 73 - DNA, 1890 bp, Flavobacterium branchiophilum
accaataaattcactaaccagtattctctctctaagaccctgcgctttgaactgattccgcaggggaaaaccttggagttcattcaagaaaaaggcctcttgtctcaggataaacagagggctgaatcttaccaagaaatgaagaaaactattgataagtttcataaatatttcattgatttagccttgtctaacgccaaattaactcacttggaaacgtatctggagttatacaacaaatctgccgaaactaagaaagaacagaaatttaaagacgatttgaaaaaagtacaggacaatctgcgtaaagaaattgtcaaatccttcagtgacggcgatgctaaaagcatttttgccattctggacaaaaaagagttgattactgtggaattagaaaagtggtttgaaaacaatgagcagaaagacatctacttcgatgagaaattcaaaactttcaccacctattttacaggatttcatcaaaaccggaagaacatgtactcagtagaaccgaactccacggccattgcgtatcgtttgatccatgagaatctgcctaaatttctggagaatgcgaaagcctttgaaaagattaagcaggtcgaatcgctgcaagtgaattttcgtgaactcatgggcgaatttggtgacgaaggtctaatcttcgttaacgaactggaagaaatgtttcagattaattactacaatgacgtgctatcgcagaacggtatcacaatctacaatagtattatctcagggttcacaaaaaacgatataaaatacaaaggcctgaacgagtatatcaataactacaaccaaacaaaggacaaaaaggataggcttccgaaactgaagcagttatacaaacagattttatctgacagaatctccctgagctttctgccggatgctttcactgatgggaagcaggttctgaaagcgattttcgatttttataagattaacttactgagctacacgattgaaggtcaagaagaatctcaaaacttactgctcttgatccgtcaaaccattgaaaatctatcatcgttcgatacgcagaaaatctacctcaaaaacgatactcacctgactacgatctctcagcaggttttcggggattttagtgtattttcaacagctctgaactactggtatgaaaccaaagtcaatccgaaattcgagacggaatattctaaggccaacgaaaaaaaacgtgagattcttgataaagctaaagccgtatttactaaacaggattacttttctattgctttcctgcaggaagttttatcggagtatatcctgaccctggatcatacatctgatatcgttaaaaaacacagcagcaattgcatcgctgactatttcaaaaaccactttgtcgccaaaaaagaaaacgaaacagacaagactttcgatttcattgctaacatcaccgcaaaataccagtgtattcagggtatcttggaaaacgccgaccaatacgaagacgaactgaaacaagatcagaagctgatcgataatttaaaattcttcttagatgcaatcctggagctgctgcacttcatcaaaccgcttcatttaaagagcgagtccattaccgaaaaggacaccgccttctatgacgtttttgaaaattattatgaagccctctccttgctgactccgctgtataatatggtacgcaattacgtaacccagaaaccatattctaccgaaaaaattaaactgaactttgaaaacgcacagctgctcaacggttgggacgcgaataaagaaggtgactacctcaccaccatcctgaaaaaagatggtaactattttctggcaattatggataagaaacataataaagcattccagaaatttcctgaagggaaagaaaat

SEQ ID NO: 74 - DNA, 2269 bp, Unknown
  Description of Unknown Uncultured bacterium sequence
aagcattggccgtaagtgcgattccggaaaggagatatacatgcaccatcatcatcaccattctttcgactctttcaccaacctgtactctctgtctaaaaccctgaaattcgaaatgcgtccggttggtaacacccagaaaatgctggacaacgcgggtgttttcgaaaaagacaaactgatccagaaaaaatacggtaaaaccaaaccgtacttcgaccgtctgcaccgtgaattcatcgaagaagcgctgaccggtgttgaactgatcggtctggacgaaaacttccgtaccctggttgactggcagaaagacaaaaaaaacaacgttgcgatgaaagcgtacgaaaactctctgcagcgtctgcgtaccgaaatcggtaaaatcttcaacctgaaagcggaagactgggttaaaaacaaatacccgatcctgggtctgaaaaacaaaaacaccgacatcctgttcgaagaagcggttttcggtatcctgaaagcgcgttacggtgaagaaaaagacaccttcatcgaagttgaagaaatcgacaaaaccggtaaatctaaaatcaaccagatctctatcttcgactcttggaaaggtttcaccggttacttcaaaaaattcttcgaaacccgtaaaaacttctacaaaaacgacggtacctctaccgcgatcgcgacccgtatcatcgaccagaacctgaaacgtttcatcgacaacctgtctatcgttgaatctgttcgtcagaaagttgacctggcggaaaccgaaaaatctttctctatctctctgtctcagttcttctctatcgacttctacaacaaatgcctgctgcaggacggtatcgactactacaacaaaatcatcggtggtgaaaccctgaaaaacggtgaaaaactgatcggtctgaacgaactgatcaaccagtaccgtcagaacaacaaagaccagaaaatcccgttcttcaaactgctggacaaacagatcctgtctgaaaaaatcctgttcctggacgaaatcaaaaacgacaccgaactgatcgaagcgctgtctcagttcgcgaaaaccgcggaagaaaaaaccaaaatcgttaaaaaactgttcgcggacttcgttgaaaacaactctaaatacgacctggcgcagatctacatctctcaggaagcgttcaacaccatctctaacaaatggacctctgaaaccgaaaccttcgcgaaatacctgttcgaagcgatgaaatctggtaaactggcgaaatacgaaaaaaaagacaactcttacaaattcccggacttcatcgcgctgtctcagatgaaatctgcgctgctgtctatctctctggaaggtcacttctggaaagaaaaatactacaaaatctctaaattccaggaaaaaaccaactgggaacagttcctggcgatcttcctgtacgaattcaactctctgttctctgacaaaatcaacaccaaagacggtgaaaccaaacaggttggttactacctgttcgcgaaagacctgcacaacctgatcctgtctgaacagatcgacatcccgaaagactctaaagttaccatcaaagacttcgcggactctgttctgaccatctaccagatggcgaaatacttcgcggttgaaaaaaaacgtgcgtggctggcggaatacgaactggactctttctacacccagccggacaccggttacctgcagttctacgacaacgcgtacgaagacatcgttcaggtttacaacaaactgcgtaactacctgaccaaaaaaccgtactctgaagaaaaatggaaactgaacttcgaaaactctaccctggcgaacggttgggacaaaaacaaagaatctgacaactctgcggttatcctgcagaaaggtggtaaatactacctgggtctgatcaccaaaggtcacaacaaaatcttcgacgaccgtttccaggaaaaattcatcgttggtatcgaaggtggtaaatacgaaaaaatcgtttacaaattcttcccggaccaggcgaaaatgttcccgaaagtttgcttctctgcgaaaggtctggaattcttccgtccgtctgaagaaatcctgcgtatctacaacaacgcggaattcaaaaaaggtgaaacctactctatcgactctatgcagaaactgatcgacttctacaaagactgcctgaccaaatacgaaggttgggcgtgctacaccttccgtcacctgaaaccgaccgaagaataccagaacaacatcggtgaattcttccgtgac

SEQ ID NO: 75 - DNA, 1056 bp, Acidamonococcus sp
acccagttcgaaggtttcaccaacctgtaccaggtttctaaaaccctgcgtttcgaactgatcccgcagggtaaaaccctgaaacacatccaggaacagggtttcatcgaagaagacaaagcgcgtaacgaccactacaaagaactgaaaccgatcatcgaccgtatctacaaaacctacgcggaccagtgcctgcagctggttcagctggactgggaaaacctgtctgcggcgatcgactcttaccgtaaagaaaaaaccgaagaaacccgtaacgcgctgatcgaagaacaggcgacctaccgtaacgcgatccacgactacttcatcggtcgtaccgacaacctgaccgacgcgatcaacaaacgtcacgcggaaatctacaaaggtctgttcaaagcggaactgttcaacggtaaagttctgaaacagctgggtaccgttaccaccaccgaacacgaaaacgcgctgctgcgttctttcgacaaattcaccacctacttctctggtttctacgaaaaccgtaaaaacgttttctctgcggaagacatctctaccgcgatcccgcaccgtatcgttcaggacaacttcccgaaattcaaagaaaactgccacatcttcacccgtctgatcaccgcggttccgtctctgcgtgaacacttcgaaaacgttaaaaaagcgatcggtatcttcgtttctacctctatcgaagaagttttctctttcccgttctacaaccagctgctgacccagacccagatcgacctgtacaaccagctgctgggtggtatctctcgtgaagcgggtaccgaaaaaatcaaaggtctgaacgaagttctgaacctggcgatccagaaaaacgacgaaaccgcgcacatcatcgcgtctctgccgcaccgtttcatcccgctgttcaaacagatcctgtctgaccgtaacaccctgtctttcatcctggaagaattcaaatctgacgaagaagttatccagtctttctgcaaatacaaaaccctgctgcgtaacgaaaacgttctggaaaccgcggaagcgctgttc

SEQ ID NO: 76 - DNA, 1098 bp, Eubacterium rectale
gtcgataatctgtgctacaaactggagttctgcccgattaaaacctcgtttatagaaaacctgatagataacggcgacctgtatctgtttcgcatcaataacaaagacttcagcagtaaatcgaccggcaccaagaaccttcatacgttatatttacaagctatattcgatgaacgtaatctgaacaatccgacaattatgctgaatgggggagcagaactgttctatcgtaaagaaagtattgagcagaaaaaccgtatcacacacaaagccggttcaattctcgtgaataaggtgtgtaaagacggtacaagcctggatgataagatacgtaatgaaatttatcaatatgagaataaatttattgataccctgtctgatgaagctaaaaaggtgttaccgaatgtcattaaaaaggaagctacccatgacattacaaaagataaacgtttcactagtgacaaattcttctttcactgccccctgacaattaattataaggaaggcgataccaagcagttcaataacgaagtgctgagttttctgcgtggaaatcctgacatcaacattatcggcattgaccgcggagagcgtaatttaatctatgtaacggttataaaccagaaaggcgagattctggattcggtttcattcaataccgtgaccaacaagagttcaaaaatcgagcagacagtcgattatgaagagaaattggcagtccgcgagaaagagaggattgaagcaaaacgttcctgggactctatctcaaaaattgcgacactaaaggaaggttatctgagcgcaatagttcacgagatctgtctgttaatgattaaacacaacgcgatcgttgtcttagagaatcttaatgcaggctttaagcgtattcgtggcggtttatcagaaaaaagtgtttatcaaaaattcgaaaaaatgttgattaacaaactgaactattttgtcagcaagaaggaatccgactggaataaaccgtctggtctgctgaatggactgcagctttcggatcagtttgaaagcttcgaaaaactgggtattcagtctggttttattttttacgtgccggctgcatatacctca

SEQ ID NO: 77 - DNA, 771 bp, Succinivibrio dextrinosolvens
aagattgatccgaccacgggcttcgccaatgttctgaatctgtcgaaggtacgcaatgttgatgcgatcaaaagctttttttctaacttcaacgaaattagttatagcaagaaagaagcccttttcaaattctcattcgatctggattcactgagtaagaaaggctttagtagctttgtgaaatttagtaagagtaaatggaacgtctacacctttggagaacgtatcataaagccaaagaataagcaaggttatcgggaggacaaaagaatcaacttgaccttcgagatgaagaagttacttaacgagtataaggtttcttttgatcttgaaaataacttgattccgaatctcacgagtgccaacctgaaggatactttttggaaagagctattctttatcttcaagactacgctgcagctccgtaacagcgttactaacggtaaagaagatgtgctcatctctccggtcaaaaatgcgaagggtgaattcttcgtttcgggaacgcataacaagactcttccgcaagattgcgatgcgaacggtgcataccatattgcgttgaaaggtctgatgatactcgaacgtaacaaccttgtacgtgaggagaaagatacgaaaaagattatggcgatttcaaacgtggattggttcgagtacgtgcagaaacgtagaggcgttctgtaagaaatcatccttagcgaaagctaaggattttttttatctgaaatgtagggagaccctcaggttaaatattcactcaggaagtta

SEQ ID NO: 78 - DNA, 738 bp, Candidatus Methanoplasma termitum
aaaatcgacccgaccaccggtttcgttaacctgttcaacacctcttctaaaaccaacgcgcaggaacgtaaagaattcctgcagaaattcgaatctatctcttactctgcgaaagacggtggtatcttcgcgttcgcgttcgactaccgtaaattcggtacctctaaaaccgaccacaaaaacgtttggaccgcgtacaccaacggtgaacgtatgcgttacatcaaagaaaaaaaacgtaacgaactgttcgacccgtctaaagaaatcaaagaagcgctgacctcttctggtatcaaatacgacggtggtcagaacatcctgccggacatcctgcgttctaacaacaacggtctgatctacaccatgtactcttctttcatcgcggcgatccagatgcgtgtttacgacggtaaagaagactacatcatctctccgatcaaaaactctaaaggtgaattcttccgtaccgacccgaaacgtcgtgaactgccgatcgacgcggacgcgaacggtgcgtacaacatcgcgctgcgtggtgaactgaccatgcgtgcgatcgcggaaaaattcgacccggactctgaaaaaatggcgaaactggaactgaaacacaaagactggttcgaattcatgcagacccgtggtgactaagaaatcatccttagcgaaagctaaggattttttttatctgaaatgtagggagaccctcaggttaaatattcactcaggaagtta

SEQ ID NO: 79 - DNA, 837 bp, Thiomicrospira sp.
gtgcggctgcattttttatgtgcctgctgcatacacgagcttctgttttacgtgccggcagattatacttcaaaaatcgatccaacaactggctttgtgaacttcctggacctgagatatcagtctgtagaaaaagctaaacaacttcttagcgattttaatgccattcgttttaacagcgttcagaattactttgaattcgaaattgactataaaaaacttactccgaaacgtaaagtcggaacccaaagtaaatgggtaatttgtacgtatggcgatgtcaggtatcagaaccgtcggaatcaaaaaggtcattgggagaccgaagaagtgaacgtgaccgaaaagctgaaggctctgttcgccagcgattcaaaaactacaactgtgatcgattacgcaaatgatgataacctgatagatgtgattttagagcaggataaagccagcttttttaaagaactgttgtggctcctgaaacttacgatgaccttacgacattccaagatcaaatcggaagatgattttattctgtcaccggtcaagaatgagcagggtgaattctatgatagtaggaaagccggcgaagtgtggccgaaagacgccgacgccaatggcgcctatcatatcgcgctcaaagggctttggaatttgcagcagattaaccagtgggaaaaaggtaaaaccctgaatctggctatcaaaaaccaggattggtttagctttatccaagagaaaccgtatcaggaatgagaaatcatccttagcgaaagctaaggattttttttatctgaaatgtagggagaccctcaggttaaatattcactcaggaagtta

SEQ ID NO: 80 - DNA, 772 bp, Candidatus Methanomethylophilus alvus
ggttatcttttatataccggcagcgttcactagtaaaatagatccgaccactggtttcgccgatctctttgccctgagtaacgttaaaaacgtagcgagcatgcgtgaattcttttccaaaatgaaatctgtcatttatgataaagctgaaggcaaattcgcattcacctttgattacttggattacaacgtgaagagcgaatgtggtcgtacgctgtggaccgtttacaccgttggtgagcgcttcacctattcccgtgtgaaccgcgaatatgtacgtaaagtccccaccgatattatctatgatgccctccagaaagcaggcattagcgtcgaaggagacttaagggacagaattgccgaaagcgatggcgatacgctgaagtctattttttacgcattcaaatacgcgctagatatgcgcgttgagaatcgcgaggaagactacattcaatcacctgtgaaaaatgcctctggggaatttttttgttcaaaaaatgctggtaaaagcctcccacaagatagcgatgcaaacggtgcatataacattgccctgaaaggtattcttcaattacgcatgctgtctgagcagtacgaccccaacgcggaatctattagacttccgctgataaccaataaagcctggctgacattcatgcagtctggcatgaagacctggaaaaattaggaaatcatccttagcgaaagctaaggattttttttatctgaaatgtagggagaccctcaggttaaatattcactcaggaagtta

SEQ ID NO: 81 - DNA, 775 bp, Porphyromonas crevioricanis
gttttatatcccggcttggaacacgagcaacatagatccgactactggatttgttaatttatttcatgcccagtatgaaaatgtagataaagcgaagagcttctttcaaaagtttgattcaattagttacaacccgaagaaagactggtttgagtttgcattcgattataaaaactttactaaaaaggctgaaggaagtcgttctatgtggatattatgcacacatggttcccgaataaagaattttagaaattcccagaagaatggtcaatgggattccgaagaattcgccttgacggaggcttttaagtctctttttgtgcgatatgagatagattataccgctgatttgaaaacagctattgtggacgaaaagcaaaaagacttcttcgtggatcttctgaagctattcaaattgacagtacagatgcgcaacagctggaaagagaaggatttggattatctaatctctcctgtagcaggggctgatggccgtttcttcgatacaagagagggaaataaaagtctgcctaaggatgcagatgccaatggagcttataatattgccctaaaaggactttgggctctacgccagattcggcaaacttcagaaggcggtaaactcaaattggcgatttccaataaggaatggctacagtttgtgcaagagagatcttacgagaaagactgagaaatcatccttagcgaaagctaaggattttttttatctgaaatgtagggagaccctcaggttaaatattcactcaggaagtta

SEQ ID NO: 82 - DNA, 787 bp, Eubacterium rectale
tttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaagaaatcatccttagcgaaagctaaggattttttttatctgaaatgtagggagaccctcaggttaaatattcactcaggaagtta

SEQ ID NO: 83 - DNA, 753 bp, Flavobacterium branchiophilum
atcgaccctacaaccggcttcgtcaattacttctatactaaatatgaaaacgtcgacaaagcaaaagcattctttgaaaagttcgaagcaatacgttttaacgctgagaaaaaatatttcgagttcgaagtcaagaaatactcagactttaaccccaaagctgagggcacacagcaagcgtggacaatctgcacctacggcgagcgcatcgaaacgaagcgtcaaaaagatcagaataacaaatttgtttcaacacctatcaacctgaccgagaagattgaagacttcttaggtaaaaatcagattgtttatggcgacggtaactgtataaaatctcaaatagcctcaaaggatgataaagcatttttcgaaacattattatattggttcaaaatgacactgcagatgcgcaatagtgagacgcgtacagatattgattatcttatcagcccggtcatgaacgacaacggtactttttacaactccagagactatgaaaaacttgagaatccaactctccccaaagatgctgatgcgaacggtgcttatcacatcgcgaaaaaaggtctgatgctgctgaacaaaatcgaccaagccgatctgactaagaaagttgacctaagcatttcaaatcgggactggttacagtttgttcaaaagaacaaatgagaaatcatccttagcgaaagctaaggattttttttatctgaaatgtagggagaccctcaggttaaatattcactcaggaagtta

SEQ ID NO: 84 - DNA, 782 bp, Unknown
  Description of Unknown Uncultured bacterium sequence
tctacacccaggcgtcttacacctctaaatctgacccggttaccggttggcgtccgcacctgtacctgaaatacttctctgcgaaaaaagcgaaagacgacatcgcgaaattcaccaaaatcgaattcgttaacgaccgtttcgaactgacctacgacatcaaagacttccagcaggcgaaagaatacccgaacaaaaccgtttggaaagtttgctctaacgttgaacgtttccgttgggacaaaaacctgaaccagaacaaaggtggttacacccactacaccaacatcaccgaaaacatccaggaactgttcaccaaatacggtatcgacatcaccaaagacctgctgacccagatctctaccatcgacgaaaaacagaacacctctttcttccgtgacttcatcttctacttcaacctgatctgccagatccgtaacaccgacgactctgaaatcgcgaaaaaaaacggtaaagacgacttcatcctgtctccggttgaaccgttcttcgactctcgtaaagacaacggtaacaaactgccggaaaacggtgacgacaacggtgcgtacaacatcgcgcgtaaaggtatcgttatcctgaacaaaatctctcagtactctgaaaaaaacgaaaactgcgaaaaaatgaaatggggtgacctgtacgtttctaacatcgactgggacaacttcgttgaaatcatccttagcgaaagctaaggattttttttatctgaaatgtagggagaccctcaggttaaatattcactcaggaagtta

SEQ ID NO: 85 - DNA, 782 bp, Acidamonococcus sp.; identical to SEQ ID NO: 84

SEQ ID NO: 86 - DNA, 1101 bp, Eubacterium rectale
gtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagc

SEQ ID NO: 87 - protein, 4 aa, Unknown
  Description of Unknown DEAD/DEAH box domain
  MOD_RES (4)..(4): Asp or His
DEAX

(19) United States
(12) Patent Application Publication
(10) Pub. No.: US 2019/0359976 A1
(43) Pub. Date: Nov. 28, 2019

(54) NOVEL ENGINEERED AND CHIMERIC NUCLEASES

(71) Applicants: The Regents of the University of Colorado, a body corporate, Denver, CO (US); Inscripta, Inc., Denver, CO (US)

(72) Inventors: Ryan T. GILL, Denver, CO (US); Andrew GARST, Boulder, CO (US); Tanya Elizabeth WARNECKE LIPSCOMB, Boulder, CO (US)

(21) Appl. No.: 16/357,443

(22) Filed: Mar. 19, 2019

Related U.S. Application Data
(63) Continuation of application No. PCT/US17/56344, filed on Oct. 12, 2017.
(60) Provisional application No. 62/483,948, filed on Apr. 10, 2017.
(60) Provisional application No. 62/407,326, filed on Oct. 12, 2016.

(51) Int. Cl. C12N 15/10; C12N 15/52; C12N 15/113; C40B 10/00; C40B 40/08; C40B 50/10; G16C 20/50

(52) U.S. Cl. CPC C12N 15/1082; C12N 15/52; C12N 15/1072; C12N 15/113; G16C 20/50; C40B 10/00; C40B 40/08; C40B 50/10; C12N 15/1089

(57) ABSTRACT

Disclosed herein are engineered nucleases and nuclease systems, including chimeric nucleases and chimeric nuclease systems. Engineered and chimeric nucleases disclosed herein include nucleic acid guided nucleases. Additionally disclosed herein are methods of generating engineered nucleases and methods of using the same.

### CROSS-REFERENCE

The present application is a continuation application of PCT/US2017/056344, filed Oct. 12, 2017, which claims priority to U.S. Provisional Application Ser. No. 62/407,326, filed Oct. 12, 2016 and U.S. Provisional Application Ser. No. 62/483,948 filed Apr. 10, 2017, the contents of each being hereby incorporated by reference in their entirety.

### SEQUENCE LISTING

The instant application contains a Sequence Listing which has been filed electronically in ASCII format and is hereby incorporated by reference in its entirety. Said ASCII copy, created on Jul. 31, 2019, is named 49022-706_301_SL.txt and is 395,941 bytes in size.

### BACKGROUND OF THE DISCLOSURE

Nucleases, including nucleic acid guided nucleases, have become important tools for research and genome engineering. The applicability of these tools can be limited by the sequence specificity requirements, expression, or delivery issues.

### SUMMARY OF THE DISCLOSURE

Disclosed herein are methods for generating a library of chimeric nuclease nucleic acid sequences, said method comprising: providing a plurality of at least a first and second nuclease nucleic acid comprising at least two domain sequences; replacing at least one of the two domain sequences of the first nuclease nucleic acid sequence with the corresponding domain sequence of the second nuclease nucleic acid sequence, thereby generating the library of chimeric nuclease nucleic acid sequences. In some embodiments, the first and second nucleic acid sequence comprise at least three domain sequences, and wherein two or more domain sequences of the first nuclease nucleic acid are replaced by the corresponding domain sequences of the second nuclease nucleic acid sequence, thereby generating the library of chimeric nuclease nucleic acid sequences. In some embodiments, replacing comprises PCR amplifying the domain sequences. In some embodiments, replacing further comprises performing an in vitro assembly method. In some embodiments, the chimeric nuclease is a chimeric nucleic acid-guided nuclease. In some embodiments, the chimeric nucleic acid-guided nuclease is capable of targeting a target nucleic acid sequence. In some embodiments, one or more of the domain sequences encodes a globular domain. In some embodiments, the one or more domain sequences encodes a modular looped out helical domain capable of mediating DNA binding. In some embodiments, one or more domain sequences encodes a globular domain capable of interacting with a displaced DNA sequence complementary to the target DNA sequence. In some embodiments, at least one nuclease sequence is from a nuclease of the Cpf1 family.

Disclosed herein are methods for generating a library of chimeric nuclease nucleic acid sequences, said method comprising: providing a plurality of at least three nuclease nucleic acids, the nucleases comprising at least three domain sequences; replacing at least one of the three domain sequences of the first nuclease nucleic acid sequence with the corresponding domain sequence of the second nuclease nucleic acid sequence, and replacing at least one of the other three domain sequences of the first nuclease nucleic acid sequence with the corresponding domain sequence of the third nuclease nucleic acid sequence, thereby generating the library of chimeric nuclease nucleic acid sequences. In some embodiments, replacing comprises PCR amplifying the domain sequences. In some embodiments, replacing further comprises performing an in vitro assembly method. In some embodiments, the chimeric nuclease is a chimeric nucleic acid-guided nuclease. In some embodiments, the chimeric nucleic acid-guided nuclease is capable of targeting a target nucleic acid sequence. In some embodiments, one or more of the domain sequences encodes a globular domain. In some embodiments, the one or more domain sequences encodes a modular looped out helical domain capable of mediating DNA binding. In some embodiments, one or more domain sequences encodes a globular domain capable of interacting with a displaced DNA sequence complementary to the target DNA sequence. In some embodiments, at least one nuclease nucleic acid is from the Cpf1 family. In some embodiments, at least two nuclease nucleic acids are from the Cpf1 family.

Disclosed herein are isolated nucleases sharing at least 85% sequence identity with a nuclease from an organism belonging to the group consisting of Priscirickettsiaceae, Thiomicrospira, and Thiomicrospira sp. XS5. In some embodiments, the isolated nuclease is a nucleic acid-guided nuclease. In some embodiments, the isolated nuclease comprises a modification or mutation compared to a corresponding wildtype sequence. In some embodiments, the isolated nuclease comprises at least 85% identity to SEQ ID No. 1. In some embodiments, the isolated nuclease comprises at least one RuvC or RuvC-like domain. In some embodiments, the isolated nuclease comprises two RuvC or RuvC-like domains. In some embodiments, the isolated nuclease comprises three RuvC or RuvC-like domain. In some embodiments, at least one of the RuvC or RuvC-like domains comprises a modification or mutation compared to a corresponding wildtype sequence. In some embodiments, the isolated nuclease comprises a RuvC I domain with at least 85% identity to the RuvC I domain of SEQ ID No. 1. In some embodiments, the isolated nuclease comprises a RuvC II domain with at least 85% identity to the RuvC II domain of SEQ ID No. 1. In some embodiments, the isolated nuclease comprises a RuvC III domain with at least 85% identity to the RuvC III domain of SEQ ID No. 1. In some embodiments, the isolated nuclease comprises a Zinc Finger or Zinc Finger-like domain. In some embodiments, the Zinc Finger or Zinc Finger-like domain comprises at least 85% identity to a Zinc Finger or Zinc Finger-like domain of SEQ ID No. 1. In some embodiments, the isolated nuclease comprises an amino acid sequence with at most 90% sequence identity to SEQ ID NO: 30. In some embodiments, the isolated nuclease comprises an amino acid sequence with at most 80% sequence identity to SEQ ID NO: 30. In some embodiments, the isolated nuclease comprises an amino acid sequence with at most 70% sequence identity to SEQ ID NO: 30. In some embodiments, the isolated nuclease comprises an amino acid sequence with at most 60% sequence identity to SEQ ID NO: 30. In some embodiments, the isolated nuclease comprises an amino acid sequence with at most 50% sequence identity to SEQ ID NO: 30. In some embodiments, the isolated nuclease comprises an amino acid sequence with at most 40% sequence identity to SEQ ID NO: 30. In some embodiments, the isolated nuclease comprises an amino acid sequence with at most 35% sequence identity to SEQ ID NO: 30. In some embodiments, the isolated nuclease is guided by a nucleic acid guide comprising at least 10 consecutive nucleotides of any one of SEQ ID NO. 13-2.

Disclosed herein are isolated nucleases sharing at least 85% sequence identity with a nuclease from an organism belonging to the group consisting of Erysipelotrichia, Enterococcaceae, Catenibacterium, Kandleria, Clostridiales, Lachnospiraceae, Dorea, Coprococcus, Enterococcus, Fructobacillus, Weisella, and Pediococcus. In some embodiments, the isolated nuclease is a nucleic acid-guided nuclease. In some embodiments, the isolated nuclease comprises a modification or mutation compared to a corresponding wildtype sequence. In some embodiments, the isolated nuclease comprises at least 85% identity to any one of SEQ ID No. 3-12. In some embodiments, the isolated nuclease comprises an RuvC or RuvC-like domain. In some embodiments, the isolated nuclease comprises at least one RuvC or RuvC-like domain. In some embodiments, the isolated nuclease comprises two RuvC or RuvC-like domains. In some embodiments, the isolated nuclease comprises three RuvC or RuvC-like domain. In some embodiments, at least one of the RuvC or RuvC-like domains comprises a modification or mutation compared to a corresponding wildtype sequence. In some embodiments, the isolated nuclease comprises a RuvC I domain with at least 85% identity to the RuvC I domain of any one of SEQ ID No. 3-12. In some embodiments, the isolated nuclease comprises a RuvC II domain with at least 85% identity to the RuvC II domain of any one of SEQ ID No. 3-12. In some embodiments, the isolated nuclease comprises a RuvC III domain with at least 85% identity to the RuvC III domain of any one of SEQ ID No. 3-12. In some embodiments, the isolated nuclease comprises a HNH or HNH-like domain. In some embodiments, the HNH or HNH-like domain comprises at least 85% identity to a HNH or HNH-like domain of any one of SEQ ID No. 3-12. In some embodiments, the isolated nuclease comprises an amino acid sequence with at most 90% sequence identity to SEQ ID NO: 31. In some embodiments, the isolated nuclease comprises an amino acid sequence with at most 80% sequence identity to SEQ ID NO: 31. In some embodiments, the isolated nuclease comprises an amino acid sequence with at most 70% sequence identity to SEQ ID NO: 31. In some embodiments, the isolated nuclease comprises an amino acid sequence with at most 60% sequence identity to SEQ ID NO: 31. In some embodiments, the isolated nuclease comprises an amino acid sequence with at most 50% sequence identity to SEQ ID NO: 31. In some embodiments, the isolated nuclease comprises an amino acid sequence with at most 40% sequence identity to SEQ ID NO: 31. In some embodiments, the isolated nuclease comprises an amino acid sequence with at most 35% sequence identity to SEQ ID NO: 31. In some embodiments, the isolated nuclease is guided by a nucleic acid guide comprising at least 10 consecutive nucleotides of any one of SEQ ID NO. 25-29, or 32-33.

Disclosed herein are engineered nucleases comprising a first fragment and a second fragment, wherein the first fragment is from a first protein and the second fragments is from a second protein, and wherein the first protein is a nuclease from an organism belonging to the group consisting of Piscirickettsiaceae, Thiomicrospira, Thiomicrospira sp. XS5, Eubacterium rectale, Succinivibrio dextrinosolvens, or any other nuclease disclosed herein. In some embodiments the first protein is a first nucleic acid-guided nuclease. In some embodiments, the engineered nuclease comprises a C-terminal fragment. In some embodiments, the first fragment comprises the C-terminal fragment. In some embodiments, the C-terminal fragment comprises a modification or mutation compared to a corresponding wildtype sequence. In some embodiments, the C-terminal fragment comprises at least 85% identity to a C-terminal fragment of SEQ ID No. 1, 2, or 50. In some embodiments, the engineered nuclease comprises an N-terminal fragment. In some embodiments, the first fragment comprises the N-terminal fragment. In some embodiments, the N-terminal fragment comprises a modification or mutation compared to a corresponding wildtype sequence. In some embodiments, the N-terminal fragment comprises at least 85% identity to an N-terminal fragment of SEQ ID No. 1, 2, or 50. In some embodiments, the engineered nuclease comprises a middle fragment. In some embodiments, the first fragment comprises the middle fragment. In some embodiments, the middle fragment comprises a modification or mutation compared to a corresponding wildtype sequence. In some embodiments, the middle fragment comprises at least 85% identity to a middle fragment of SEQ ID No. 1, 2, or 50. In some embodiments, the engineered nuclease comprises a polypeptide fragment or linker region. In some embodiments, the first fragment comprises the polypeptide fragment or linker region. In some embodiments, the polypeptide fragment or linker region comprises a modification or mutation compared to a corresponding wildtype sequence. In some embodiments, the polypeptide fragment or linker region comprises at least 85% identity to a polypeptide fragment or linker domain of SEQ ID No. 1, 2, or 50. In some embodiments, the engineered nuclease comprises an RuvC or RuvC-like domain. In some embodiments, the first fragment comprises the RuvC or RuvC-like domain. In some embodiments, the engineered nuclease comprises at least one RuvC or RuvC-like domain. In some embodiments, the first fragment comprises the at least one RuvC or RuvC-like domain. In some embodiments, the engineered nuclease comprises two RuvC or RuvC-like domains. In some embodiments, the first fragment comprises the two RuvC or RuvC-like domains. In some embodiments, the engineered nuclease comprises three RuvC or RuvC-like domain. In some embodiments, the first fragment comprises the three RuvC or RuvC-like domain. In some embodiments, at least one of the RuvC or RuvC-like domains comprises a modification or mutation compared to a corresponding wildtype sequence. In some embodiments, the engineered nuclease comprises a RuvC I domain with at least 85% identity to the RuvC I domain of SEQ ID No. 1, 2, or 50. In some embodiments, the first fragment comprises the RuvC I domain. In some embodiments, the engineered nuclease comprises a RuvC II domain with at least 85% identity to the RuvC II domain of SEQ ID No. 1, 2, or 50. In some embodiments, the first fragment comprises the RuvC II domain. In some embodiments, the engineered nuclease comprises a RuvC III domain with at least 85% identity to the RuvC III domain of SEQ ID No. 1, 2, or 50. In some embodiments, the first fragment comprises the RuvC III domain. In some embodiments, the engineered nuclease comprises a Zinc Finger or Zinc Finger-like domain. In some embodiments, the first fragment comprises the Zinc Finger or Zinc Finger-like domain. In some embodiments, the Zinc Finger or Zinc Finger-like domain comprises at least 85% identity to a Zinc Finger or Zinc Finger-like domain of SEQ ID No. 1, 2, or 50. In some embodiments, the first nucleic acid-guided nuclease is a Cpf1 ortholog. In some embodiments, the first nucleic acid-guided nuclease comprises an amino acid sequence with at most 90% sequence identity to SEQ ID NO: 30. In some embodiments, the first nucleic acid-guided nuclease comprises an amino acid sequence with at most 80% sequence identity to SEQ ID NO: 30. In some embodiments, the first nucleic acid-guided nuclease comprises an amino acid sequence with at most 70% sequence identity to SEQ ID NO: 30. In some embodiments, the first nucleic acid-guided nuclease comprises an amino acid sequence with at most 60% sequence identity to SEQ ID NO: 30. In some embodiments, the first nucleic acid-guided nuclease comprises an amino acid sequence with at most 50% sequence identity to SEQ ID NO: 30. In some embodiments, the first nucleic acid-guided nuclease comprises an amino acid sequence with at most 40% sequence identity to SEQ ID NO: 30. In some embodiments, the first nucleic acid-guided nuclease comprises an amino acid sequence with at most 35% sequence identity to SEQ ID NO: 30. In some embodiments, the engineered nuclease comprises an amino acid sequence with at most 90% sequence identity to SEQ ID NO: 30. In some embodiments, the engineered nuclease comprises an amino acid sequence with at most 80% sequence identity to SEQ ID NO: 30. In some embodiments, the engineered nuclease comprises an amino acid sequence with at most 70% sequence identity to SEQ ID NO: 30. In some embodiments, the engineered nuclease comprises an amino acid sequence with at most 60% sequence identity to SEQ ID NO: 30. In some embodiments, the engineered nuclease comprises an amino acid sequence with at most 50% sequence identity to SEQ ID NO: 30. In some embodiments, the engineered nuclease comprises an amino acid sequence with at most 40% sequence identity to SEQ ID NO: 30. In some embodiments, the engineered nuclease comprises an amino acid sequence with at most 35% sequence identity to SEQ ID NO: 30. In some embodiments, the second protein is a second nucleic acid-guided nuclease. In some embodiments, the second nucleic acid-guided nuclease is from an organism belonging to the group consisting of Piscirickettsiaceae, Thiomicrospira, Eubacterium rectale, and Succinivibrio dextrinosolvens. In some embodiments, the second nucleic acid-guided nuclease is from an organism belonging to the group consisting of Succinivibrio dextrinosolvens, Candidatus Methanoplasma termitum, Candidatus Methanomethylophilus alvus, Porphyromonas crevioricanis, Flavobacterium branchiophilum, Lachnospiraceae bacterium COE1, Prevotella brevis ATCC 19188, Smithella sp. SCADC, Moraxella bovoculi, Synergistes jonesii, Bacteroidetes oral taxon 274, Francisella tularensis, Leptospira inadai serovar Lyme str. 10, Acidomonococcus sp. crystal structure (5B43). In some embodiments, the second nucleic acid-guided nuclease is from an organism belonging to the group consisting of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In some embodiments, the engineered nuclease is guided by a nucleic acid guide comprising at least 10 consecutive nucleotides of any one of SEQ ID NO. 13-24, or 30. In some embodiments, an engineered nuclease further comprises a third fragment from a third protein. In some embodiments, the third protein is a nuclease.

Disclosed herein are engineered nucleases comprising a first fragment and a second fragment, wherein the first fragment is from a first protein and the second fragments is from a second protein, and wherein the first protein is a nuclease from an organism belonging to the group consisting of Erysipelotrichia, Enterococcacease, Catenibacterium, Kandleria, Clostridiales, Lachnospiraceae, Dorea, Coprococcus, Enterococcus, Fructobacillus, Weissella, Pediococcus. In some embodiments, the first protein is a first nucleic acid-guided nuclease. In some embodiments, the engineered nuclease comprises a C-terminal fragment. In some embodiments, the first fragment comprises the C-terminal fragment. In some embodiments, the C-terminal fragment comprises a modification or mutation compared to a corresponding wildtype sequence. In some embodiments, the C-terminal fragment comprises at least 85% identity to a C-terminal fragment of any one of SEQ ID No. 3-12. In some embodiments, the engineered nuclease comprises an N-terminal fragment. In some embodiments, the first fragment comprises the N-terminal fragment. In some embodiments, the N-terminal fragment comprises a modification or mutation compared to a corresponding wildtype sequence In some embodiments, the N-terminal fragment comprises at least 85% identity to an N-terminal fragment of any one of SEQ ID No. 3-12. In some embodiments, the engineered nuclease comprises a middle fragment. In some embodiments, the first fragment comprises the middle fragment. In some embodiments, the middle fragment comprises a modification or mutation compared to a corresponding wildtype sequence In some embodiments, the middle fragment comprises at least 85% identity to a middle fragment of any one of SEQ ID No. 3-12. In some embodiments, the engineered nuclease comprises a polypeptide fragment or linker region. In some embodiments, the first fragment comprises the polypeptide fragment or linker region. In some embodiments, the polypeptide fragment or linker region comprises a modification or mutation compared to a corresponding wildtype sequence In some embodiments, the polypeptide fragment or linker region comprises at least 85% identity to a polypeptide fragment or linker domain of any one of SEQ ID No. 3-12. In some embodiments, the engineered nuclease comprises an RuvC or RuvC-like domain. In some embodiments, the first fragment comprises the RuvC or RuvC-like domain. In some embodiments, the engineered nuclease comprises at least one RuvC or RuvC-like domain. In some embodiments, the first fragment comprises the at least one RuvC or RuvC-like domain. In some embodiments, the engineered nuclease comprises two RuvC or RuvC-like domains. In some embodiments, the first fragment comprises the two RuvC or RuvC-like domains. In some embodiments, the engineered nuclease comprises three RuvC or RuvC-like domain. In some embodiments, the first fragment comprises the three RuvC or RuvC-like domain. In some embodiments, at least one of the RuvC or RuvC-like domains comprises a modification or mutation compared to a corresponding wildtype sequence. In some embodiments, the engineered nuclease comprises a RuvC I domain with at least 85% identity to the RuvC I domain of any one of SEQ ID No. 3-12. In some embodiments, the first fragment comprises the RuvC I domain. In some embodiments, the engineered nuclease comprises a RuvC II domain with at least 85% identity to the RuvC II domain of any one of SEQ ID No. 3-12. In some embodiments, the first fragment comprises the RuvC II domain. In some embodiments, the engineered nuclease comprises a RuvC III domain with at least 85% identity to the RuvC III domain of any one of SEQ ID No. 3-12. In some embodiments, the first fragment comprises the RuvC III domain. In some embodiments, the engineered nuclease comprises a HNH or HNH-like domain. In some embodiments, the first fragment comprises the HNH or HNH-like domain. In some embodiments, the HNH or HNH-like domain comprises at least 85% identity to a HNH or HNH-like domain of any one of SEQ ID No. 3-12. In some embodiments, the first nucleic acid-guided nuclease is a Cas9 ortholog. In some embodiments, the first nucleic acid-guided nuclease comprises an amino acid sequence with at most 90% sequence identity to SEQ ID NO: 31. In some embodiments, the first nucleic acid-guided nuclease comprises an amino acid sequence with at most 80% sequence identity to SEQ ID NO: 31. In some embodiments, the first nucleic acid-guided nuclease comprises an amino acid sequence with at most 70% sequence identity to SEQ ID NO: 31. In some embodiments, the first nucleic acid-guided nuclease comprises an amino acid sequence with at most 60% sequence identity to SEQ ID NO: 31. In some embodiments, the first nucleic acid-guided nuclease comprises an amino acid sequence with at most 50% sequence identity to SEQ ID NO: 31. In some embodiments, the first nucleic acid-guided nuclease comprises an amino acid sequence with at most 40% sequence identity to SEQ ID NO: 31. In some embodiments, the first nucleic acid-guided nuclease comprises an amino acid sequence with at most 35% sequence identity to SEQ ID NO: 31. In some embodiments, the engineered nuclease comprises an amino acid sequence with at most 90% sequence identity to SEQ ID NO: 31. In some embodiments, the engineered nuclease comprises an amino acid sequence with at most 80% sequence identity to SEQ ID NO: 31. In some embodiments, the engineered nuclease comprises an amino acid sequence with at most 70% sequence identity to SEQ ID NO: 31. In some embodiments, the engineered nuclease comprises an amino acid sequence with at most 60% sequence identity to SEQ ID NO: 31. In some embodiments, the engineered nuclease comprises an amino acid sequence with at most 50% sequence identity to SEQ ID NO: 31. In some embodiments, the engineered nuclease comprises an amino acid sequence with at most 40% sequence identity to SEQ ID NO: 31. In some embodiments, the engineered nuclease comprises an amino acid sequence with at most 35% sequence identity to SEQ ID NO: 31. In some embodiments, the second protein is a second nucleic acid-guided nuclease. In some embodiments, the second nucleic acid-guided nuclease is from an organism belonging to the group consisting of Erysipelotrichia, Enterococcacease, Catenibacterium, Kandleria, Clostridiales, Lachnospiraceae, Dorea, Coprococcus, Enterococcus, Fructobacillus, Weissella, Pediococcus. In some embodiments, the second nucleic acid-guided nuclease is from an organism belonging to the group consisting of Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, Pediococcus acidilactici. In some embodiments, the second nucleic acid-guided nuclease is from an organism belonging to the group consisting of Lactobacillus curvatus, Streptococcus pyogenes, Lactobacillus versmoldensis, Filifactor alocis ATCC 35896. In some embodiments, the second nucleic acid-guided nuclease is from an organism belonging to the group consisting of Streptococcus, Lactobacillus, Staphylococcus, Roseburia, Filifactor, Eubacterium, Corynebacter, Bacteroides, Flaviivola, Flavobacterium, Parvibaculum, Azospirillum, Gluconacetobacter, Sutterella, Neisseria, Legionella, Nitratifractor, Campylobacter, Sphaerochaeta, Treponema, Mycoplasma. In some embodiments, the engineered nuclease is guided by a nucleic acid guide comprising at least 10 consecutive nucleotides of any one of SEQ ID NO. 25-29, or 31-33. In some embodiments, an engineered nuclease further comprises a third fragment from a third protein. In some embodiments, the third protein is a nuclease.

Disclosed herein are nucleic acid molecules encoding any isolated nuclease or engineered nuclease disclosed herein. In some embodiments, the nucleic acid molecule is codon-optimized for expression in a eukaryotic cell. In some embodiments, the nucleic acid molecule is codon-optimized for expression in a prokaryotic cell. In some embodiments, the nucleic acid molecule is synthesized.

Disclosed herein are vectors comprising a nucleic acid molecule encoding any isolated nuclease or engineered nuclease disclosed herein. In some embodiments, the vector further comprises a regulatory element operable in a eukaryotic cell operably linked to the nucleic acid molecules encoding the isolated nuclease or engineered nuclease. In some embodiments, the vector further comprises a regulatory element operable in a prokaryotic cell operably linked to the nucleic acid molecules encoding the isolated nuclease or engineered nuclease.

Disclosed herein are engineered nuclease systems that bind to at least one target sequence in a cell containing a DNA molecule comprising said target, wherein the engineered nuclease system comprises any isolated nuclease or engineered nuclease disclosed herein and a guide nucleic acid. In some embodiments, when introduced into said cell having said DNA molecule, the isolated nuclease or engineered nuclease cleaves said target sequence. In some embodiments, the guide nucleic acid is encoded on a nucleic acid. In some embodiments, the nucleic acid encoding said guide nucleic acid is a synthetic nucleic acid. In some embodiments, the guide nucleic acid comprises a single nucleic acid molecule. In some embodiments, the guide nucleic acid comprises two nucleic acid molecules. In some embodiments, the system further comprises template DNA for insertion into the cleaved strand of the DNA molecule.

Disclosed herein are methods of altering the sequence of at least one gene product in a cell containing a DNA molecule having a target sequence and encoding said gene product comprising introducing into said cell an engineered nuclease system comprising one or more vectors comprising: a) at least one nucleotide sequence encoding a guide nucleic acid that hybridizes with the target sequence, and b) a nucleotide sequence encoding any isolated nuclease or engineered nuclease disclosed herein, whereby said guide nucleic acid hybridizes to the target sequence and said isolated nuclease or engineered nuclease cleaves the DNA molecule; whereby the sequence of said at least one gene product is altered. In some embodiments, said guide nucleic acid comprises one polynucleotide molecule. In some embodiments, said guide nucleic acid comprises two polynucleotide molecules. In some embodiments, the method further comprises a first regulatory element operably linked to the at least one nucleotide sequence encoding a guide nucleic acid that hybridizes with the target sequence. In some embodiments, the method further comprises a second regulatory element operably linked to the nucleotide sequence encoding the isolated nuclease or engineered nuclease. In some embodiments, said first or second regulatory elements are selected from the group consisting of a promoter, terminator, enhancers, or stabilizing element. In some embodiments, components (a) and (b) are located the same vector of the system. In some embodiments, components (a) and (b) are located different vectors of the system. In some embodiments, the different vectors are introduced into said cell concurrently. In some embodiments, the different vectors are introduced into said cell sequentially. In some embodiments, the method further comprises inserting template DNA into a cleaved strand of the DNA molecule. In some embodiments, said cell is a eukaryotic cell. In some embodiments, said cell is a prokaryotic cell.

Disclosed herein are cells comprising any isolated nuclease or engineered nuclease disclosed herein.

Disclosed herein are cells comprising any nucleic acid molecule disclosed herein.

Disclosed herein are cells comprising any vector disclosed herein.

Disclosed herein are cells comprising any engineered nuclease system disclosed herein.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 depicts an example chimeric nuclease library construction scheme.

FIG. 2 depicts an example chimeric nuclease library constructions scheme.

### DETAILED DESCRIPTION OF THE DISCLOSURE

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present disclosure provides engineered nuclease systems comprising a nucleic acid-targeting system, wherein nucleic acid is DNA or RNA, and in some aspects may also refer to DNA-RNA hybrids or derivatives thereof, and wherein the system refers collectively to transcripts and other elements involved in the expression of or directing the activity of engineered nuclease genes, which may include sequences encoding an engineered nuclease protein and a guide nucleic acid as disclosed herein.

Methods, systems, vectors, polynucleotides, and compositions described herein may be used in various nucleic acids-targeting applications, altering or modifying synthesis of a gene product, such as a protein, nucleic acids cleavage, nucleic acids editing, nucleic acids splicing; trafficking of target nucleic acids, tracing of target nucleic acids, isolation of target nucleic acids, visualization of target nucleic acids, etc. Aspects of the invention also encompass methods and uses of the compositions and systems described herein in genome engineering, or gene regulation, e.g. for altering or manipulating the expression of one or more genes or the one or more gene products, in prokaryotic or eukaryotic cells, in vitro, in vivo or ex vivo.

### Novel Nucleases

Aspects of the invention relate to novel nucleic acid-guided nucleases and systems. In a further embodiment the nucleases are functional in prokaryotic or eukaryotic cells for in vitro, in vivo or ex vivo applications. The present disclosure relates to systems, methods and compositions used for genome engineering involving sequence targeting, such as genome perturbation or gene-editing, that relate to nucleic acid-guided nuclease systems and components thereof. In advantageous embodiments, a nuclease is a nucleic acid-guided nuclease.

Disclosed herein are nucleic acid-guided nucleases. Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof. Suitable nucleic acid-guided nucleases can be from an organism from a genus which includes but is not limited to Thiomicrospira, Succinivibrio, Candidatus, Porphyromonas, Acidomonococcus, Prevotella, Smithella, Moraxella, Synergistes, Francisella, Leptospira, Catenibacterium, Kandleria, Clostridium, Dorea, Coprococcus, Enterococcus, Fructobacillus, Weissella, Pediococcus, Corynebacter, Sutterella, Legionella, Treponema, Roseburia, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Alicyclobacillus, Brevibacilus, Bacillus, Bacteroidetes, Brevibacilus, Carnobacterium, Clostridiaridium, Clostridium, Desulfonatronum, Desulfovibrio, Helcococcus, Leptotrichia, Listeria, Methanomethyophilus, Methylobacterium, Opitutaceae, Paludibacter, Rhodobacter, Sphaerochaeta, Tuberibacillus, and Campylobacter. Species of organism of such a genus can be as otherwise herein discussed. Suitable nucleic acid-guided nucleases can be from an organism from a genus or unclassified genus within a kingdom which includes but is not limited to Firmicute, Actinobacteria, Bacteroidetes, Proteobacteria, Spirochates, and Tenericutes. Suitable nucleic acid-guided nucleases can be from an organism from a genus or unclassified genus within a phylum which includes but is not limited to Erysipelotrichia, Clostridia, Bacilli, Actinobacteria, Bacteroidetes, Flavobacteria, Alphaproteobacteria, Betaproteobacteria, Gammaproteobacteria, Deltaproteobacteria, Epsilonproteobacteria, Spirochaetes, and Mollicutes. Suitable nucleic acid-guided nucleases can be from an organism from a genus or unclassified genus within an order which includes but is not limited to Clostridiales, Lactobacillales, Actinomycetales, Bacteroidales, Flavobacteriales, Rhizobiales, Rhodospirillales, Burkholderiales, Neisseriales, Legionellales, Nautiliales, Campylobacterales, Spirochaetales, Mycoplasmatales, and Thiotrichales. Suitable nucleic acid-guided nucleases can be from an organism from a genus or unclassified genus within a family which includes but is not limited to Lachnospiraceae, Enterococcaceae, Leuconostocaceae, Lactobacillaceae, Streptococcaceae, Peptostreptococcaceae, Staphylococcaceae, Eubacteriaceae, Corynebacterineae, Bacteroidaceae, Flavobacterium, Cryomoorphaceae, Rhodobiaceae, Rhodospirillaceae, Acetobacteraceae, Sutterellaceae, Neisseriaceae, Legionellaceae, Nautiliaceae, Campylobacteraceae, Spirochaetaceae, Mycoplasmataceae, Pisciririckettsiaceae, and Francisellaceae.

Other nucleic acid-guided nucleases suitable for use in the methods, systems, and compositions of the present disclosure include those derived from an organism such as, but not limited to, Thiomicrospira sp. XS5, Eubacterium rectale, Succinivibrio dextrinosolvens, Candidatus Methanoplasma termitum, Candidatus Methanomethylophilus alvus, Porphyromonas crevioricanis, Flavobacterium branchiophilum, Acidomonococcus sp., Lachnospiraceae bacterium COE1, Prevotella brevis ATCC 19188, Smithella sp. SCADC, Moraxella bovoculi, Synergistes jonesii, Bacteroidetes oral taxon 274, Francisella tularensis, Leptospira inadai serovar Lyme str. 10, Acidomonococcus sp. crystal structure (5B43) S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, Porphyromonas macacae, Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, Pediococcus acidilactici, Lactobacillus curvatus, Streptococcus pyogenes, Lactobacillus versmoldensis, and Filifactor alocis ATCC 35896.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.).

In some instances, a nuclease disclosed herein comprises an amino acid sequence comprising at least 50% amino acid identity to any one of SEQ ID NO: 1-12, or 50-66. In some instances, a nuclease comprises an amino acid sequence comprising at least 10%, 20%, 30%, 40%, 50%, 60%, 70%, 80%, 90%, 95%, greater than 90%, or 100% amino acid identity to any one of SEQ ID NO: 1-12 or 50-66. In some instances, a nuclease comprises an amino acid sequence comprising at most 10%, 20%, 30%, 40%, 50%, 60%, 70%, 80%, 90%, or more than 90% amino acid identity to any one of SEQ ID NO: 30-31. In some instances, an engineered nuclease comprises an amino acid sequence comprising at most 50% amino acid identity to any one of SEQ ID NO: 30-31.

### Engineered Nucleases

Aspects of the invention relate to the engineering of novel nucleic acid-guided nucleases and systems. In further embodiments the engineered nucleases are functional in prokaryotic or eukaryotic cells for in vitro, in vivo or ex vivo applications. The present disclosure relates to the engineering and optimization of systems, methods and compositions used for genome engineering involving sequence targeting, such as genome perturbation or gene-editing, that relate to nucleic acid-guided nuclease systems and components thereof. In advantageous embodiments, the nucleic acid-guided nuclease is an engineered nuclease, e.g. an engineered Cas9 homolog or ortholog, an engineered Cpf1 homolog of ortholog, or an engineered chimeric nuclease comprising fragments of one or more Cas9 or Cpf1 homologs or orthologs.

Disclosed herein are engineered nucleases. Engineered nucleases can include nucleic acid guided nucleases, chimeric nuclease, and nuclease fusions. Such engineered nucleases include, but are not limited to, an engineered Cas9 homolog or ortholog, an engineered Cpf1 homolog of ortholog, a chimeric engineered nuclease comprising fragments of one or more Cas9 or Cpf1 homologs or orthologs, a chimeric engineered nuclease comprising fragments of one or more nucleic acid guided nucleases, or any combination thereof. Engineered nucleases or chimeric nucleases disclosed herein can comprise any nuclease disclosed in U.S. application Ser. No. 15/631,989 filed Jun. 23, 2017, or U.S. application Ser. No. 15/632,001 filed Jun. 23, 2017, the contents of each of which are herein incorporated by reference in their entirety.

Chimeric and/or Fusion Engineered Nucleases

Chimeric engineered nuclease as disclosed herein can comprise one or more fragments or domains, and the fragments or domains can be of a nuclease, such as nucleic acid-guided nuclease, orthologs of organisms of genuses, species, or other phylogenetic groups disclosed herein. Advantageously, the fragments can be from nuclease orthologs of different species. A chimeric engineered nuclease can be comprised of fragments or domains from at least two different nucleases. A chimeric engineered nuclease can be comprised of fragments or domains from nucleases from at least two different species. A chimeric engineered nuclease can be comprised of fragments or domains from at least 2, 3, 4, 5, 6, 7, 8, 9, 10, or more different nucleases or nucleases from different species. In some cases, an chimeric engineered nuclease comprises more than one fragment or domain from one nuclease, wherein the more than one fragment or domain are separated by fragments or domains from a second nuclease. In some examples, a chimeric engineered nuclease comprises 2 fragments, each from a different protein or nuclease. In some examples, a chimeric engineered nuclease comprises 3 fragments, each from a different protein or nuclease. In some examples, a chimeric engineered nuclease comprises 4 fragments, each from a different protein or nuclease. In some examples, a chimeric engineered nuclease comprises 5 fragments, each from a different protein or nuclease. In some examples, a chimeric engineered nuclease comprises 3 fragments, wherein at least one fragment is from a different protein or nuclease. In some examples, a chimeric engineered nuclease comprises 4 fragments, wherein at least one fragment is from a different protein or nuclease. In some examples, a chimeric engineered nuclease comprises 5 fragments, wherein at least one fragment is from a different protein or nuclease.

Junctions between fragments or domains from different nucleases or species can but need not to occur in stretches of unstructured regions. Unstructured regions may include regions which are exposed within a protein structure and/or are not conserved within various nuclease orthologs.

In some instances, an engineered nuclease comprises an amino acid sequence comprising at least 50% amino acid identity to an exemplary wild-type nuclease (e.g., SEQ ID NO: 1-12 or 50-66). In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66). In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66). In some instances, an engineered nuclease comprises an amino acid sequence comprising at most 50% amino acid identity to an exemplary wild-type nuclease (e.g., SEQ ID NO: 1-12 or 50-66).

An engineered nuclease can comprise one or more domains including an RuvC domain, RuvC-like domain, HNH domain, HNH-like domain, Zinc Finger domain, Zinc Finger-like domain, globular domain, modular looped out helical domain, and any combination thereof. RuvC domains or RuvC-like domains can comprise RuvC I domains, RuvC II domains, and/or RuvC III domains. In some cases an engineered nucleases comprises one, two, three, four, five, or more than five RuvC domains. In some cases, an engineered nuclease comprises three RuvC domains. In some cases, an engineered nuclease comprises an RuvC I, RuvC II, and RuvC III domains.

An engineered nuclease, including a chimeric engineered nuclease, can comprise one or more RuvC or RuvC-like domains. An RuvC or RuvC-like domain may be substituted or inserted with an RuvC or RuvC-like domain, or fragment thereof, derived from another nuclease from a different species. Non-native RuvC or RuvC-like domains may be derived from any suitable organism, such as those disclosed herein. In some cases, the nuclease and/or RuvC or RuvC-like domain may be derived from prokaryotic organisms, archaea, bacteria, protists (e.g., Thiomicrospira sp. XS5, Eubacterium rectale, or Succinivibrio dextrinosolvens). In some cases, the nuclease and/or RuvC or RuvC-like domain may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified RuvC or RuvC-like domain. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified RuvC or RuvC-like domain. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified RuvC or RuvC-like domain. In some instances, an engineered nuclease comprises an amino acid sequence comprising at most 50% amino acid identity to an exemplary wild-type nuclease (e.g., SEQ ID NO: 1-12 or 50-66), and a modified RuvC or RuvC-like domain.

An engineered nuclease, including a chimeric engineered nuclease, can comprise one or more HNH or HNH-like domains. An HNH or HNH-like domain may be substituted or inserted with an HNH or HNH-like domain, or fragment thereof, derived from another nuclease from a different species. Non-native HNH or HNH-like domains may be derived from any suitable organism, such as those disclosed herein. In some cases, the nuclease and/or HNH or HNH-like domain may be derived from prokaryotic organisms, archaea, bacteria, protists (e.g., Thiomicrospira sp. XS5, Eubacterium rectale, or Succinivibrio dextrinosolvens). In some cases, the nuclease and/or HNH or HNH-like domain may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified HNH or HNH-like domain. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified HNH or HNH-like domain. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified HNH or HNH-like domain. In some instances, an engineered nuclease comprises an amino acid sequence comprising at most 50% amino acid identity to an exemplary wild-type nuclease (e.g., SEQ ID NO: 1-12 or 50-66), and a modified HNH or HNH-like domain.

An engineered nuclease, including a chimeric engineered nuclease, can comprise one or more Zinc Finger or Zinc Finger-like domains. A Zinc Finger or Zinc Finger-like domain may be substituted or inserted with a Zinc Finger or Zinc Finger-like domain, or fragment thereof, derived from another nuclease from a different species. Non-native Zinc Finger or Zinc Finger-like domains may be derived from any suitable organism, such as those disclosed herein. In some cases, the nuclease and/or Zinc Finger or Zinc Finger-like domain may be derived from prokaryotic organisms, archaea, bacteria, protists (e.g., Thiomicrospira sp. XS5, Eubacterium rectale, or Succinivibrio dextrinosolvens). In some cases, the Zinc Finger or Zinc Finger-like domain may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified Zinc Finger or Zinc Finger-like domain. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified Zinc Finger or Zinc Finger-like domain. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified Zinc Finger or Zinc Finger-like domain. In some instances, an engineered nuclease comprises an amino acid sequence comprising at most 50% amino acid identity to an exemplary wild-type nuclease (e.g., SEQ ID NO: 1-12 or 50-66), and a modified Zinc Finger or Zinc Finger-like domain.

An engineered nuclease, including a chimeric engineered nuclease, can comprise one or more globular domains. A globular domain may be substituted or inserted with a globular domain, or fragment thereof, derived from another nuclease from a different species. Non-native globular domains may be derived from any suitable organism, such as those disclosed herein. In some cases, the globular domain may be derived from prokaryotic organisms, archaea, bacteria, protists (e.g., Thiomicrospira sp. XS5, Eubacterium rectale, or Succinivibrio dextrinosolvens). In some cases, the globular domain may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified globular domain. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified globular domain. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified globular domain. In some instances, an engineered nuclease comprises an amino acid sequence comprising at most 50% amino acid identity to an exemplary wild-type nuclease (e.g., SEQ ID NO: 1-12 or 50-66), and a modified globular domain.

An engineered nuclease, including a chimeric engineered nuclease, can comprise one or more modular looped out helical domains. A globular domain may be substituted or inserted with a modular looped out helical domain, or fragment thereof, derived from another nuclease from a different species. Non-native modular looped out helical domains may be derived from any suitable organism, such as those disclosed herein. In some cases, the modular looped out helical domain may be derived from prokaryotic organisms, archaea, bacteria, protists (e.g., Thiomicrospira sp. XS5, Eubacterium rectale, or Succinivibrio dextrinosolvens). In some cases, the modular looped out helical domain may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified modular looped out helical domain. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified modular looped out helical domain. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified modular looped out helical domain. In some instances, an engineered nuclease comprises an amino acid sequence comprising at most 50% amino acid identity to an exemplary wild-type nuclease (e.g., SEQ ID NO: 1-12 or 50-66), and a modified modular looped out helical domain.

An engineered nuclease, including a chimeric engineered nuclease, can comprise N-terminal fragment. An N-terminal fragment may be substituted or inserted with an N-terminal fragment derived from another nuclease from a different species. Non-native N-terminal fragments may be derived from any suitable organism, such as those disclosed herein. In some cases, the nuclease and/or N-terminal fragment may be derived from prokaryotic organisms, archaea, bacteria, protists (e.g., Thiomicrospira sp. XS5, Eubacterium rectale, or Succinivibrio dextrinosolvens). In some cases, the nuclease and/or N-terminal fragment may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified N-terminal fragment. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified N-terminal fragment. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified N-terminal fragment. In some instances, an engineered nuclease comprises an amino acid sequence comprising at most 50% amino acid identity to an exemplary wild-type nuclease (e.g., SEQ ID NO: 1-12 or 50-66), and a modified N-terminal fragment.

An engineered nuclease, including a chimeric engineered nuclease, can comprise middle fragment. A middle fragment may be substituted or inserted with a middle fragment derived from another nuclease from a different species. Non-native middle fragments may be derived from any suitable organism, such as those disclosed herein. In some cases, the nuclease and/or middle fragment may be derived from prokaryotic organisms, archaea, bacteria, protists (e.g., Thiomicrospira sp. XS5, Eubacterium rectale, or Succinivibrio dextrinosolvens). In some cases, the nuclease and/or N-terminal fragment may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified middle fragment. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified middle fragment. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified middle fragment. In some instances, an engineered nuclease comprises an amino acid sequence comprising at most 50% amino acid identity to an exemplary wild-type nuclease (e.g., SEQ ID NO: 1-12 or 50-66), and a modified middle fragment.

An engineered nuclease, including a chimeric engineered nuclease, can comprise C-terminal fragment. A C-terminal fragment may be substituted or inserted with a C-terminal fragment derived from another nuclease from a different species. Non-native C-terminal fragments may be derived from any suitable organism, such as those disclosed herein. In some cases, the nuclease and/or C-terminal fragment may be derived from prokaryotic organisms, archaea, bacteria, protists (e.g., Thiomicrospira sp. XS5, Eubacterium rectale, or Succinivibrio dextrinosolvens). In some cases, the nuclease and/or C-terminal fragment may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified C-terminal fragment. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified C-terminal fragment. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified C-terminal fragment. In some instances, an engineered nuclease comprises an amino acid sequence comprising at most 50% amino acid identity to an exemplary wild-type nuclease (e.g., SEQ ID NO: 1-12 or 50-66), and a modified C-terminal fragment.

An engineered nuclease, including a chimeric engineered nuclease, can comprise a polypeptide fragment and/or linker region. A polypeptide fragment and/or linker region may be substituted or inserted with a polypeptide fragment and/or linker region derived from another nuclease from a different species. Non-native polypeptide fragment and/or linker region may be derived from any suitable organism, such as those disclosed herein. In some cases, the nuclease and/or polypeptide fragment and/or linker region may be derived from prokaryotic organisms, archaea, bacteria, protists (e.g., Thiomicrospira sp. XS5, Eubacterium rectale, or Succinivibrio dextrinosolvens). In some cases, the nuclease and/or polypeptide fragment and/or linker region may be derived from prokaryotic organisms, archaea, bacteria, protists (e.g., Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici).

In some instances, an engineered nuclease comprises an amino acid sequence comprising at least 50% amino acid identity to an exemplary wild-type nuclease (e.g., SEQ ID NO: 1-12 or 50-66), and a modified polypeptide fragment and/or linker region. In some instances, an engineered nuclease comprises an amino acid sequence comprising at least 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified polypeptide fragment and/or linker region. In some instances, an engineered nuclease comprises an amino acid sequence comprising at most 10%, 20%, 30%, 40%, 50%, 60%, 70%, 80%, 90%, or more than 90% amino acid identity to an exemplary wild-type nuclease (e.g., SEQ ID NO: 1-12 or 50-66), and a modified polypeptide fragment and/or linker region. In some instances, an engineered nuclease comprises an amino acid sequence comprising at most 50% amino acid identity to an exemplary wild-type nuclease (e.g., SEQ ID NO: 1-12 or 50-66), and a modified polypeptide fragment and/or linker region.

Engineered nucleases as disclosed herein can comprise one or more fragments. Such fragments can include N-terminal fragments, C-terminal fragments, and middle fragments. Fragments can comprise functional domains, nonfunctional domains, linker sequence, regulatory elements, promoters, terminators, enhancers, untranslated regions, coding sequence, introns, exons, or other polynucleotide sequence. Fragments can but need not include all or a portion of one or more domains. Such domains can include functional domains including a nuclease domain, HNH domain, RuvC domain, RuvC-like domain, RuvC I domain, RuvC II domain, RuvC III domain, Zinc Finger domain, Zinc Finger-like domain, DNase domain, RNase domain, or other known nucleic acid cleavage domain or nucleic acid binding domain. More examples of functional domains include but are not limited to Fok1, VP64, P65, HSF1, MyoD1, translational initiator, translational activator, translational repressor, nucleases, in particular ribonucleases, a spliceosome, beads, a light inducible/controllable domain, a chemically inducible/controllable domain, or domain conferring methylase activity, demethylase activity, transcription release factor activity, histone modification activity, RNA cleavage activity, DNA cleavage activity, nucleic acid binding activity, and molecular switches. Other non-limiting examples of functional domains include regulatory domains, nucleases, transposases or methylases, to modify endogenous chromosomal sequences, transcription factor repressor or activator domains such as KRAB and VP16, co-repressor and co-activator domains, DNA methyl transferases, histone acetyltransferases, histone deacetylases, and DNA cleavage domains such as the cleavage domain from the endonuclease FokI.

In some instances, an engineered nuclease is modified such that it comprises a non-native sequence, for example that alters it from the allele or sequence it was derived from. The non-native sequence can also include one or more additional proteins, protein domains, subdomains or polypeptides. For example, an engineered nuclease may be fused with any suitable additional nonnative nucleic acid binding proteins and/or domains, including but not limited to transcription factor domains, nuclease domains, nucleic acid polymerizing domains. A non-native sequence can comprise a sequence of a nucleic acid-guided nuclease and/or an other nuclease homologue or ortholog.

A non-native sequence can confer new functions to the engineered nuclease. These functions can include for example, DNA methylation, DNA damage, DNA repair, modification of a target polypeptide associated with target DNA (e.g., a histone, a DNA-binding protein, etc.), leading to, for example, histone methylation, histone acetylation, histone ubiquitination, and the like. Other functions conferred can include methyltransferase activity, demethylase activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, and demyristoylation activity, or any combination thereof.

In some embodiments, an engineered nuclease as disclosed herein is part of a fusion protein comprising one or more heterologous protein domains (e.g. about or more than about 1, 2, 3, 4, 5, 6, 7, 8, 9, 10, or more domains in addition to nuclease domains). An engineered nuclease fusion protein may comprise any additional protein sequence, and optionally a linker sequence between any two domains. Examples of protein domains that may be fused to an engineered nuclease include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). An engineered nuclease may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising an engineered nuclease are described in US20110059502, incorporated herein by reference. In some embodiments, a tagged engineered nuclease is used to identify the location of a target sequence.

In some instances, an engineered nuclease as disclosed herein is a fusion protein comprising a chromatin-remodeling enzyme or functional domain thereof. Without wishing to be bound by theory, an engineered nuclease fusion protein as described herein may provide improved accessibility to regions of highly-structured DNA. Non-limiting examples of chromatin-remodeling enzymes that can be linked to a nucleic-acid guided nuclease may include: histone acetyl transferases (HATs), histone deacetylases (HDACs), histone methyltransferases (HMTs), chromatin remodeling complexes, and transcription activator-like (Tal) effector proteins. Histone deacetylases may include HDAC1, HDAC2, HDAC3, HDAC4, HDAC5, HDAC6, HDAC7, HDAC8, HDAC9, HDAC10, HDAC11, sirtuin 1, sirtuin 2, sirtuin 3, sirtuin 4, sirtuin 5, sirtuin 6, and sirtuin 7. Histone acetyl transferases may include GCN5, PCAF, Hat1, Elp3, Hpa2, Hpa3, ATF-2, Nut1, Esa1, Sas2, Sas3, Tip60, MOF, MOZ, MORF, HBO1, p300, CBP, SRC-1, ACTR, TIF-2, SRC-3, TAFII250, TFIIIC, Rtt109, and CLOCK. Histone methyltransferases may include ASH1L, DOT1L, EHMT1, EHMT2, EZH1, EZH2, MLL, MLL2, MLL3, MLL4, MLL5, NSD1, PRDM2, SET, SETBP1, SETD1A, SETD1B, SETD2, SETD3, SETD4, SETD5, SETD6, SETD7, SETD8, SETD9, SETDB1, SETDB2, SETMAR, SMYD1, SMYD2, SMYD3, SMYD4, SMYD5, SUV39H1, SUV39H2, SUV420H1, and SUV420H2. Chromatin-remodeling complexes may include SWI/SNF, ISWI, NuRD/Mi-2/CHD, INO80 and SWR1.

In some instances, an engineered nuclease as disclosed herein is a cell-cycle-dependent nuclease. A cell-cycle dependent nuclease generally includes a targeted nuclease as described herein linked to an enzyme that leads to degradation of the targeted nuclease during G1 phase of the cell cycle, and expression of the targeted nuclease during G2/M phase of the cell cycle. Such cell-cycle dependent expression may, for example, bias the expression of the nuclease in cells where homology-directed repair (HDR) is most active (e.g., during G2/M phase). In some cases, the nuclease is covalently linked to cell-cycle regulated protein such as one that is actively degraded during G1 phase of the cell cycle and is actively expressed during G2/M phase of the cell cycle. In a non-limiting example, the cell-cycle regulated protein is Geminin. Other non-limiting examples of cell-cycle regulated proteins may include: Skp2.

### Protein Modifications and Engineering

The terms “non-naturally occurring” or “engineered” are used interchangeably and indicate the involvement of the hand of man and/or woman. The terms, when referring to nucleic acid molecules or polypeptides mean that the nucleic acid molecule or the polypeptide is at least substantially free from at least one other component with which they are naturally associated in nature and as found in nature.

Engineered nucleases, as disclosed herein, can be modified or can comprise modifications. A modification can comprise modifications to an amino acid of the engineered nuclease. A modification can alter the primary amino acid sequence and/or the secondary, tertiary, and quaternary amino acid structure. In some cases, some amino acid sequences of an engineered nuclease of the invention can be varied without a significant effect on the structure or function of the protein. The type of modification or mutation may be completely unimportant if the alteration occurs in some regions (e.g. a non-critical) of the protein. In some cases, depending upon the location of the replacement, the modification or mutation may not have a major effect on the biological properties of the resulting variant. For example, properties and functions of the engineered nuclease can be of the same type as a wild-type nuclease. In some cases, the modification or mutation can critically impact the structure and/or function of the engineered nuclease.

Amino acids in an engineered nuclease of the present invention that are essential for function can be identified by methods such as site-directed mutagenesis, alanine-scanning mutagenesis, protein structure analysis, nuclear magnetic resonance, photoaffinity labeling, and electron tomography, high-throughput screening, ELISAs, biochemical assays, binding assays, cleavage assays (e.g., Surveyor assay), reporter assays, and the like.

Screens can be used to engineer or optimize an engineered nuclease. For example, a screen can be set up to screen for the effect of mutations in a region of the engineered nuclease. For example, a screen can be set up to test modifications of the highly basic patch on the affinity for RNA structure (e.g., guide nucleic acid), or processing capability (e.g., target sequence cleavage). For example, a screen can be set up to test various permutations of chimeric engineered nuclease combinations. Exemplary screening methods can include but are not limited to, protein sequence activity relationship mapping, cell sorting methods, mRNA display, phage display, and directed evolution.

The location of where to modify an engineered nuclease can be determined using sequence and/or structural alignment. Sequence alignment can identify regions of a polypeptide that are similar and/or dissimilar (e.g., conserved, not conserved, hydrophobic, hydrophilic, etc). In some instances, a region in the sequence of interest that is similar to other sequences is suitable for modification. In some instances, a region in the sequence of interest that is dissimilar from other sequences is suitable for modification. For example, sequence alignment can be performed by database search, pairwise alignment, multiple sequence alignment, genomic analysis, motif finding, benchmarking, and/or programs such as BLAST, CS-BLAST, HHPRED, psi-BLAST, LALIGN, PyMOL, and SEQALN. Structural alignment can be performed by programs such as Dali, PHYRE, Chimera, COOT, O, and PyMOL. Alignment can be performed by database search, pairwise alignment, multiple sequence alignment, genomic analysis, motif finding, or bench marking, or any combination thereof.

In some cases, the modification can comprise a conservative modification. A conservative amino acid change can involve substitution of one of a family of amino acids which are related in their side chains (e.g, cysteine/serine)

In some cases amino acid changes in the engineered nucleases disclosed herein are non-conservative amino acid changes, (i.e., substitutions of dissimilar charged or uncharged amino acids). A non-conservative amino acid change can involve substitution of one of a family of amino acids which may be unrelated in their side chains or a substitution that alters biological activity of the engineered nuclease.

The present disclosure provides methods, compositions, and/or systems, for modifying or using modified engineered nucleases, including chimeric engineered nucleases, engineered nucleic acid-guided nucleases, and chimeric engineered nucleic acid-guided nucleases. Modifications may include any covalent or non-covalent modification to engineered nucleases as disclosed herein. In some cases, this may include chemical modifications to one or more fragments, regions, domains, or sequences of the engineered nuclease. In some cases, modifications may include conservative or non-conservative amino acid substitutions of the engineered nuclease. In some cases, modifications may include the addition, deletion or substitution of any portion of the engineered nuclease with amino acids, peptides, or domains that are not found in the native nuclease. In some cases, one or more non-native domains may be added, deleted, or substituted in the engineered nuclease. In some cases the engineered nuclease may exist as a fusion protein or a chimeric protein.

In some cases, the present disclosure provides for the engineering of nucleases to recognize a desired guide nucleic acid or target sequence with desired enzyme specificity and/or activity. Modifications to an engineered nuclease can be performed through protein engineering. Protein engineering can include fusing functional domains to such engineered nuclease which can be used to modify the functional state of the overall engineered nuclease or the actual target nucleic acid sequence, such as a target sequence in a host cell.

Engineered nucleases as disclosed herein, including chimeric engineered nucleases, can comprise one or more modifications, including mutations, compared to a wildtype nuclease, or in the case of chimeric engineered nucleases, one or more mutations compared to wildtype sequences of fragments or domains of which the chimeric engineered nuclease is comprised. Such one or more mutations can be generated or engineered into a coding region, such as an open reading frame, exon, or sequence encoding a functional domain, or non-coding region, such as a 5′ UTR, promoter, intron, terminator, or 3′ UTR.

One or more mutations may be engineered into an engineered nuclease in order to reduce, enhance, add functionality, remove functionality, or any combination thereof. For example, one or more mutations may be engineered in order to reduce or eliminate nucleic acid cleavage function. In another example, one or more mutations may be engineered in order to reduce or eliminate off-target effects. It is to be understood that mutated engineered nucleases, including chimeric engineered nucleases, as described herein may be used in any of the methods according to the invention as described herein.

It will be appreciated that any of the functionalities described herein may be engineered into an engineered nucleic acid-guided nuclease from other orthologs, including chimeric enzymes comprising fragments from multiple orthologs. Examples of such orthologs are described elsewhere herein. Thus, chimeric enzymes may comprise fragments of nucleic acid-guided nucleases, such as CRISPR enzyme orthologs or homologs. In some examples, mutants can be generated which lead to inactivation of the enzyme or which modify the double strand nuclease to nickase activity. In some embodiments, this information is used to develop engineered nucleases with reduced off-target effects. Reduced off-target effects can be achieved by altering binding properties between the engineered nuclease and a guide nucleic acid or target sequence.

In some instances, one or more specific domains, regions, or structural elements of an engineered nuclease can be modified or mutated together. Modifications to an engineered nuclease may occur, but are not limited to nuclease elements such as regions that recognize or bind to nucleic acid target sequence. Modifications to an engineered nuclease may occur, but are not limited to nucleic acid-guided nuclease elements such as regions that bind or recognize a guide nucleic acid. Such binding or recognition elements may include a RuvC domain, a RuvC-like domain, a HNH domain, a HNH-like domain, a Zinc Finger domain, a Zinc Finger-like domain, a nuclease domain, a nucleic acid binding domain, a nucleic acid cleavage domain, a guide nucleic acid binding domain, or any combination thereof. Modifications may be made to additional domains, structural elements, sequence or amino acids within the engineered nuclease.

In certain embodiments, altered activity of an engineered nuclease comprises increased targeting efficiency or decreased off-target binding. In certain embodiments, the altered activity of the engineered nuclease comprises modified cleavage activity. In certain embodiments, the altered activity comprises altered binding property as to the guide nucleic acid or the target polynucleotide, altered binding kinetics as to the guide nucleic acid or the target polynucleotide, or altered binding specificity as to the guide nucleic acid or the target polynucleotide compared to off-target polynucleotide.

In certain embodiments, altered activity comprises increased targeting efficiency or decreased off-target binding. In certain embodiments, the altered activity comprises modified cleavage activity. In certain embodiments, the altered activity comprises increased cleavage activity as to the target polynucleotide. In certain embodiments, the altered activity comprises decreased cleavage activity as to the target polynucleotide. In certain embodiments, the altered activity comprises decreased cleavage activity as to off-target polynucleotide. In certain embodiments, the altered activity comprises increased cleavage activity as to off-target polynucleotide.

In certain embodiments, the altered activity comprises increased cleavage activity as to the target polynucleotide. In certain embodiments, the altered activity comprises decreased cleavage activity as to the target polynucleotide. In certain embodiments, the altered activity comprises decreased cleavage activity as to off-target polynucleotide. In certain embodiments, the altered activity comprises increased cleavage activity as to off-target polynucleotide. Accordingly, in certain embodiments, there is increased specificity for target polynucleotide as compared to off-target polynucleotide. In other embodiments, there is reduced specificity for target polynucleotide as compared to off-target polynucleotide.

In some aspects of the invention, the engineered nuclease comprises a modification that alters association of the protein with the guide nucleic acid, or a strand of the target polynucleotide, or a strand of off-target polynucleotide. In some aspects of the invention, the engineered nuclease comprises a modification that alters formation of the engineered nuclease complex.

In certain embodiments, the engineered nuclease comprises a modification that alters targeting of the guide nucleic acid to the target polynucleotide. In certain embodiments, the modification comprises a mutation in a region of the engineered nuclease that associates with the guide nucleic acid. In certain embodiments, the modification comprises a mutation in a region of the engineered nuclease that associates with a strand of the target polynucleotide. In certain embodiments, the modification comprises a mutation in a region of the engineered nuclease that associates with a strand of the off-target polynucleotide. In certain embodiments, the modification or mutation comprises decreased positive charge in a region of the engineered nuclease that associates with the guide nucleic acid, or a strand of the target polynucleotide, or a strand of off-target polynucleotide. In certain embodiments, the modification or mutation comprises decreased negative charge in a region of the engineered nuclease that associates with the guide nucleic acid, or a strand of the target polynucleotide, or a strand of off-target polynucleotide. In certain embodiments, the modification or mutation comprises increased positive charge in a region of the engineered nuclease that associates with the guide nucleic acid, or a strand of the target polynucleotide, or a strand of off-target polynucleotide. In certain embodiments, the modification or mutation comprises increased negative charge in a region of the engineered nuclease that associates with the guide nucleic acid, or a strand of the target polynucleotide, or a strand of off-target polynucleotide. In certain embodiments, the modification or mutation increases steric hindrance between the engineered nuclease and the guide nucleic acid, or a strand of the target polynucleotide, or a strand of off-target polynucleotide. In certain embodiments, the modification or mutation comprises a substitution of one or more amino acid residues, such as Lys, His, Arg, Glu, Asp, Ser, Gly, or Thr. In certain embodiments, the modification or mutation comprises a substitution with one or more amino acid residues, such as a Gly, Ala, Ile, Glu, or Asp. In certain embodiments, the modification or mutation comprises an amino acid substitution in a binding groove.

A modification may comprise modification of one or more amino acid residues of the engineered nuclease compared to a wild type nuclease, or in the case of a chimeric engineered nuclease, compared to wildtype sequences of fragments or domains of which the chimeric engineered enzyme comprises. In any such engineered nuclease, a modification may comprise modification of one or more amino acid residues located in a region which comprises residues which are positively charged in the corresponding unmodified nuclease, fragment, or domain. A modification may comprise modification of one or more amino acid residues which are positively charged in the corresponding unmodified nuclease, fragment, or domain. A modification may comprise modification of one or more amino acid residues which are not positively charged in the corresponding unmodified nuclease, fragment, or domain. A modification may comprise modification of one or more amino acid residues which are uncharged in the unmodified nuclease, fragment, or domain. A modification may comprise modification of one or more amino acid residues which are negatively charged in the unmodified nuclease, fragment, or domain. A modification may comprise modification of one or more amino acid residues which are hydrophobic in the unmodified nuclease, fragment, or domain. A modification may comprise modification of one or more amino acid residues which are polar in the unmodified nuclease, fragment, or domain. A modification may comprise modification of one or more residues located in a groove. A modification may comprise modification of one or more residues located outside of a groove. A modification may comprise a modification of one or more residues wherein the one or more residues comprises arginine, histidine or lysine.

In any of the engineered nucleases disclosed herein, the engineered nuclease may be modified by mutation of said one or more residues. In some cases, the mutation comprises substitution of a residue in the corresponding unmodified nuclease, fragment, or domain with an alanine residue. In some cases a mutation comprises substitution of a residue in the corresponding unmodified nuclease, fragment, or domain with aspartic acid or glutamic acid. In some cases, a mutation comprises substitution of a residue in the corresponding unmodified nuclease, fragment, or domain with serine, threonine, asparagine or glutamine. In some cases, a mutation comprises substitution of a residue in the corresponding unmodified nuclease, fragment, or domain with alanine, glycine, isoleucine, leucine, methionine, phenylalanine, tryptophan, tyrosine or valine. In some cases, a mutation comprises substitution of a residue in the corresponding unmodified nuclease, fragment, or domain with a polar amino acid residue. In some cases, a mutation comprises substitution of a residue in the corresponding unmodified nuclease, fragment, or domain with an amino acid residue which is not a polar amino acid residue. In some cases, a mutation comprises substitution of a residue in the corresponding unmodified nuclease, fragment, or domain with a negatively charged amino acid residue. In some cases, a mutation comprises substitution of a residue in the corresponding unmodified nuclease, fragment, or domain with an amino acid residue which is not a negatively charged amino acid residue. In some cases, a mutation comprises substitution of a residue in the corresponding unmodified nuclease, fragment, or domain with an uncharged amino acid residue. In some cases, a mutation comprises substitution of a residue in the corresponding unmodified nuclease, fragment, or domain with an amino acid residue which is not an uncharged amino acid residue. In some cases, a mutation comprises substitution of a residue in the corresponding unmodified nuclease, fragment, or domain with a hydrophobic amino acid residue. In some cases, a mutation comprises substitution of a residue in the corresponding unmodified nuclease, fragment, or domain with an amino acid residue which is not a hydrophobic amino acid residue.

Where an engineered nuclease comprises one or more mutations in one or more domains, the one or more additional mutations may be in a domain such as, though not limited to, RuvCI, RuvCII, RuvCIII, HNH, HNH-like, RuvC, RuvC-like, Zinc Finger, Zinc Finger-like, or any other functional domain or linker sequence within the engineered nuclease.

A mutation may result in a change that may comprise a change in any kinetic parameter of the engineered nuclease. The mutation may result in a change that may comprise a change in any thermodynamic parameter of the engineered nuclease. The mutation may result in in a change that may comprise a change in the surface charge, surface area buried, and/or folding kinetics of the engineered nuclease and/or enzymatic action of the engineered nuclease.

A mutation may result in a change that may comprise a change in dissociation constant (K_{d}) of binding between an engineered nuclease and a target sequence and/or guide nucleic acid. The change in K_{d} of binding between an engineered nuclease and a target sequence and/or guide nucleic acid may be more than 1000-fold, more than 500-fold, more than 100-fold, more than 50-fold, more than 25-fold, more than 10-fold, more than 5-fold, more than 4-fold, more than 3-fold, more than 2-fold higher or lower than the K_{d} of binding between a non-mutated nuclease and a target nucleic acid and/or guide nucleic acid. The change in K_{d} of binding between an engineered nuclease and a target sequence and/or guide nucleic acid may be less than 1000-fold, less than 500-fold, less than 100-fold, less than 50-fold, less than 25-fold, less than 10-fold, less than 5-fold, less than 4-fold, less than 3-fold, less than 2-fold higher or lower than the K_{d} of binding of binding between a non-mutated an nuclease and a target sequence and/or guide nucleic acid.

A mutation of an engineered nuclease can also change the kinetics of the enzymatic action of the engineered nuclease. The mutation may result in a change that may comprise a change in the Michaelis constant (Kₘ) of the engineered nuclease. The change in Kₘ of the engineered nuclease may be more than 1000-fold, more than 500-fold, more than 100-fold, more than 50-fold, more than 25-fold, more than 10-fold, more than 5-fold, more than 4-fold, more than 3-fold, more than 2-fold higher or lower than the Kₘ of a wild-type nuclease. The change in Kₘ of an engineered nuclease may be less than 1000-fold, less than 500-fold, less than 100-fold, less than 50-fold, less than 25-fold, less than 10-fold, less than 5-fold, less than 4-fold, less than 3-fold, less than 2-fold higher or lower than the Kₘ of a wild-type nuclease.

A mutation of an engineered nuclease may result in a change that may comprise a change in the turnover of the engineered nuclease. The change in the turnover of the engineered nuclease protein may be more than 1000-fold, more than 500-fold, more than 100-fold, more than 50-fold, more than 25-fold, more than 10-fold, more than 5-fold, more than 4-fold, more than 3-fold, more than 2-fold higher or lower than the turnover of a wild-type nuclease. The change in the turnover of an engineered nuclease may be less than 1000-fold, less than 500-fold, less than 100-fold, less than 50-fold, less than 25-fold, less than 10-fold, less than 5-fold, less than 4-fold, less than 3-fold, less than 2-fold higher or lower than the turnover of a wild-type nuclease.

A mutation may result in a change that may comprise a change in the free energy (ΔG) of the enzymatic action of an engineered nuclease. The change in the ΔG of the engineered nuclease may be more than 1000-fold, more than 500-fold, more than 100-fold, more than 50-fold, more than 25-fold, more than 10-fold, more than 5-fold, more than 4-fold, more than 3-fold, more than 2-fold higher or lower than the ΔG of a wild-type nuclease. The change in the turnover of an engineered nuclease may be less than 1000-fold, less than 500-fold, less than 100-fold, less than 50-fold, less than 25-fold, less than 10-fold, less than 5-fold, less than 4-fold, less than 3-fold, less than 2-fold higher or lower than the ΔG of a wild-type nuclease.

A mutation may result in a change that may comprise a change in the maximum rate of reaction (Vₘₐₓ) of the enzymatic action of an engineered nuclease. The change in the Vₘₐₓ of an engineered nuclease may be more than 1000-fold, more than 500-fold, more than 100-fold, more than 50-fold, more than 25-fold, more than 10-fold, more than 5-fold, more than 4-fold, more than 3-fold, more than 2-fold higher or lower than the Vₘₐₓ of a wild-type nuclease. The change in the turnover of an engineered nuclease may be less than 1000-fold, less than 500-fold, less than 100-fold, less than 50-fold, less than 25-fold, less than 10-fold, less than 5-fold, less than 4-fold, less than 3-fold, less than 2-fold higher or lower than the Vₘₐₓ of a wild-type nuclease.

Other amino acid alterations may also include amino acids with glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue. In some cases an engineered nuclease may also include allelic variants and species variants.

Truncations of regions which do not affect functional activity of an engineered nuclease may be engineered. Truncations of regions which do affect functional activity of an engineered nuclease may be engineered. A truncation may comprise a truncation of less than 5, less than 10, less than 15, less than 20, less than 25, less than 30, less than 35, less than 40, less than 45, less than 50, less than 60, less than 70, less than 80, less than 90, less than 100 or more amino acids. A truncation may comprise a truncation of more than 5, more than 10, more than 15, more than 20, more than 25, more than 30, more than 35, more than 40, more than 45, more than 50, more than 60, more than 70, more than 80, more than 90, more than 100 or more amino acids. A truncation may comprise truncation of about 5%, 10%, 15%, 20%, 25%, 30%, 35%, 40%, 45%, 50%, 55%, 60%, 65%, 70%, 75%, 80%, 85%, 90%, 95% or 100% of an engineered nuclease.

Deletions of regions which do not affect functional activity of an engineered nuclease may be engineered. Deletions of regions which do affect functional activity of an engineered nuclease may be engineered. A deletion can comprise a deletion of less than 5, less than 10, less than 15, less than 20, less than 25, less than 30, less than 35, less than 40, less than 45, less than 50, less than 60, less than 70, less than 80, less than 90, less than 100 or more amino acids. A deletion may comprise a deletion of more than 5, more than 10, more than 15, more than 20, more than 25, more than 30, more than 35, more than 40, more than 45, more than 50, more than 60, more than 70, more than 80, more than 90, more than 100 or more amino acids. A deletion may comprise deletion of about 5%, 10%, 15%, 20%, 25%, 30%, 35%, 40%, 45%, 50%, 55%, 60%, 65%, 70%, 75%, 80%, 85%, 90%, 95% or 100% of an engineered nuclease. A deletion can occur at the N-terminus, the C-terminus, or at any region in the polypeptide chain.

An engineered nuclease can comprise a RuvC domain or an RuvC-like domain. In some cases, an engineered nuclease comprises one, two, three, four, five, or more than five RuvC or RuvC-like domains. In some cases, an engineered nuclease comprises three RuvC or RuvC-like domains. In any of these cases, one or more of the RuvC or RuvC domains can be mutated or modified.

A RuvC or RuvC-like domain of an engineered nuclease may be modified. In some cases, an RuvC or RuvC-like domain may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with an RuvC or RuvC-like domain of an exemplary wild-type nuclease (e.g., SEQ ID NO: 1-12 or 50-66). An RuvC or RuvC-like domain may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with an RuvC or RuvC-like domain of an exemplary wild-type nuclease (e.g., SEQ ID NO: 1-12 or 50-66).

In some cases, modifications to an RuvC or RuvC-like domain may include but are not limited to individual amino acid modifications, as described herein. In some cases, modification to an RuvC or RuvC-like domain may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of an RuvC or RuvC-like domain. Modifications may include modifications to at most 1, 2, 3, 4, 5, 6, 7, 8, 9, 10, 11, 12, 13, 14, 15, 16, 17, 18, 19, 20 or more amino acids of an RuvC or RuvC-like domain. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an RuvC or RuvC-like domain. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an RuvC or RuvC-like domain.

In some cases, modifications to an RuvC or RuvC-like domain may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an RuvC or RuvC-like domain. In some cases, modifications to an RuvC or RuvC-like domain may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the an RuvC or RuvC-like domain.

In some cases, modifications to an RuvC or RuvC-like domain may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease RuvC or RuvC-like domain. In some cases, modifications to an RuvC or RuvC-like domain sequences may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease RuvC or RuvC-like domain.

Modifications to an RuvC or RuvC-like domain may include substitution or addition with one or more amino acid residues. In some cases, the RuvC or RuvC-like domain may be replaced or fused with other suitable nucleic acid binding domains. A nucleic acid-binding domain can comprise RNA. There can be a single nucleic acid-binding domain. Examples of nucleic acid-binding domains can include, but are not limited to, a helix-turn-helix domain, a zinc finger domain, a leucine zipper (bZIP) domain, a winged helix domain, a winged helix turn helix domain, a helix-loop-helix domain, a HMG-box domain, a Wor3 domain, an immunoglobulin domain, a B3 domain, a TALE domain, a Zinc-finger domain, a RNA-recognition motif domain, a double-stranded RNA-binding motif domain, a double-stranded nucleic acid binding domain, a single-stranded nucleic acid binding domains, a KH domain, a PUF domain, a RGG box domain, a DEAD/DEAH box domain, (SEQ ID NO: 87), a PAZ domain, a Piwi domain, a cold-shock domain, a RNAseH domain, a HNH domain, a RuvC-like domain, a RAMP domain, a Cas5 domain, and a Cas6 domain.

An engineered nuclease can comprise an HNH domain or an HNH-like domain. In some cases, an engineered nuclease comprises one, two, three, four, five, or more than five HNH domain or an HNH-like domains. In any of these cases, one or more of the HNH domain or an HNH-like domains can be mutated or modified.

A HNH domain or an HNH-like domain of an engineered nuclease may be modified. In some cases, an HNH domain or an HNH-like domain may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with an HNH domain or an HNH-like domain of an exemplary wild-type nuclease (e.g., SEQ ID NO: 1-12 or 50-66). An HNH domain or an HNH-like domain may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with an HNH domain or an HNH-like domain of an exemplary wild-type nuclease (e.g., SEQ ID NO: 1-12 or 50-66).

In some cases, modifications to an HNH domain or an HNH-like domain may include but are not limited to individual amino acid modifications, as described herein. In some cases, modification to an HNH domain or an HNH-like domain may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of an HNH domain or an HNH-like domain. Modifications may include modifications to at most 1, 2, 3, 4, 5, 6, 7, 8, 9, 10, 11, 12, 13, 14, 15, 16, 17, 18, 19, 20 or more amino acids of an HNH domain or an HNH-like domain. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an HNH domain or an HNH-like domain. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an HNH domain or an HNH-like domain.

In some cases, modifications to an HNH domain or an HNH-like domain may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an HNH domain or an HNH-like domain. In some cases, modifications to an HNH domain or an HNH-like domain may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the an HNH domain or an HNH-like domain.

In some cases, modifications to an HNH or HNH-like domain may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease HNH domain or an HNH-like domain. In some cases, modifications to an HNH domain or an HNH-like domain sequences may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease HNH domain or an HNH-like domain.

Modifications to a HNH or HNH-like domain may include substitution or addition with one or more amino acid residues. In some cases, the HNH domain may be replaced or fused with other suitable nucleic acid binding domains. A nucleic acid-binding domain can comprise RNA. There can be a single nucleic acid-binding domain. Examples of nucleic acid-binding domains can include, but are not limited to, a helix-turn-helix domain, a zinc finger domain, a leucine zipper (bZIP) domain, a winged helix domain, a winged helix turn helix domain, a helix-loop-helix domain, a HMG-box domain, a Wor3 domain, an immunoglobulin domain, a B3 domain, a TALE domain, a Zinc-finger domain, a RNA-recognition motif domain, a double-stranded RNA-binding motif domain, a double-stranded nucleic acid binding domain, a single-stranded nucleic acid binding domains, a KH domain, a PUF domain, a RGG box domain, a DEAD/DEAH box domain (SEQ ID NO: 87), a PAZ domain, a Piwi domain, and a cold-shock domain, a RNAseH domain, a HNH domain, a RuvC-like domain, a RAMP domain, a Cas5 domain, a Cas6 domain.

An engineered nuclease can comprise a Zinc Finger domain or a Zinc Finger-like domain. In some cases, an engineered nuclease comprises one, two, three, four, five, or more than five Zinc Finger domain or an Zinc Finger-like domain. In any of these cases, one or more of the Zinc Finger domain or a Zinc Finger-like domain can be mutated or modified.

A Zinc Finger domain or a Zinc Finger-like domain of an engineered nuclease may be modified. In some cases, a Zinc Finger domain or an Zinc Finger-like domain may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a Zinc Finger domain or a Zinc Finger-like domain of an exemplary wild-type nuclease (e.g., SEQ ID NO: 1-12 or 50-66). A Zinc Finger domain or a Zinc Finger-like domain may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a Zinc Finger domain or an Zinc Finger-like domain of an exemplary wild-type nuclease (e.g., SEQ ID NO: 1-12 or 50-66).

In some cases, modifications to a Zinc Finger domain or a Zinc Finger-like domain may include but are not limited to individual amino acid modifications, as described herein. In some cases, modification to a Zinc Finger domain or a Zinc Finger-like domain may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of a Zinc Finger domain or a Zinc Finger-like domain. Modifications may include modifications to at most 1, 2, 3, 4, 5, 6, 7, 8, 9, 10, 11, 12, 13, 14, 15, 16, 17, 18, 19, 20 or more amino acids of a Zinc Finger domain or a Zinc Finger-like domain. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a Zinc Finger domain or an Zinc Finger-like domain. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a Zinc Finger domain or an Zinc Finger-like domain.

In some cases, modifications to a Zinc Finger domain or an Zinc Finger-like domain may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a Zinc Finger domain or an Zinc Finger-like domain. In some cases, modifications to a Zinc Finger domain or an Zinc Finger-like domain may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the a Zinc Finger domain or an Zinc Finger-like domain.

In some cases, modifications to a Zinc Finger domain or an Zinc Finger-like domain may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a Zinc Finger domain or an Zinc Finger-like domain. In some cases, modifications to a Zinc Finger domain or an Zinc Finger-like domain sequences may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a Zinc Finger domain or an Zinc Finger-like domain.

Modifications to a Zinc Finger or Zinc Finger-like domain may include substitution or addition with one or more amino acid residues. In some cases, the Zinc Finger domain may be replaced or fused with other suitable nucleic acid binding domains. A nucleic acid-binding domain can comprise RNA. There can be a single nucleic acid-binding domain. Examples of nucleic acid-binding domains can include, but are not limited to, a helix-turn-helix domain, a zinc finger domain, a leucine zipper (bZIP) domain, a winged helix domain, a winged helix turn helix domain, a helix-loop-helix domain, a HMG-box domain, a Wor3 domain, an immunoglobulin domain, a B3 domain, a TALE domain, a Zinc-finger domain, a RNA-recognition motif domain, a double-stranded RNA-binding motif domain, a double-stranded nucleic acid binding domain, a single-stranded nucleic acid binding domains, a KH domain, a PUF domain, a RGG box domain, a DEAD/DEAH box domain (SEQ ID NO: 87), a PAZ domain, a Piwi domain, and a cold-shock domain, a RNAseH domain, a HNH domain, a RuvC-like domain, a RAMP domain, a Cas5 domain, a Cas6 domain.

A globular domain of an engineered nuclease may be modified. In some cases, a globular domain may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a globular domain or a of an exemplary wild-type nuclease (e.g., SEQ ID NO: 1-12 or 50-66). A globular domain may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a globular domain of an exemplary wild-type nuclease (e.g., SEQ ID NO: 1-12 or 50-66).

In some cases, modifications to a globular domain may include but are not limited to individual amino acid modifications, as described herein. In some cases, modification to a globular domain may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of a globular domain. Modifications may include modifications to at most 1, 2, 3, 4, 5, 6, 7, 8, 9, 10, 11, 12, 13, 14, 15, 16, 17, 18, 19, 20 or more amino acids of a globular domain. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a globular domain. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a globular domain.

In some cases, modifications to a globular domain may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a globular domain. In some cases, modifications to a globular domain may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the a globular domain.

In some cases, modifications to a globular domain may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a globular domain. In some cases, modifications to a globular domain sequences may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a globular domain.

Modifications to a globular domain may include substitution or addition with one or more amino acid residues. In some cases, a globular domain is capable of interacting with a displaced DNA sequence complementary to a target sequence. In some cases, the globular domain may be replaced or fused with other suitable nucleic acid binding domains, such as other suitable domains capable of interacting with a displaced DNA sequence complementary to a target sequence.

A modular looped out helical domain of an engineered nuclease may be modified. In some cases, a globular domain may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a modular looped out helical domain or a of an exemplary wild-type nuclease (e.g., SEQ ID NO: 1-12 or 50-66). A modular looped out helical domain may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a modular looped out helical domain of an exemplary wild-type nuclease (e.g., SEQ ID NO: 1-12 or 50-66).

In some cases, modifications to a modular looped out helical domain may include but are not limited to individual amino acid modifications, as described herein. In some cases, modification to a modular looped out helical domain may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of a modular looped out helical domain. Modifications may include modifications to at most 1, 2, 3, 4, 5, 6, 7, 8, 9, 10, 11, 12, 13, 14, 15, 16, 17, 18, 19, 20 or more amino acids of a modular looped out helical domain. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a modular looped out helical domain. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a modular looped out helical domain.

In some cases, modifications to a modular looped out helical domain may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a modular looped out helical domain. In some cases, modifications to a modular looped out helical domain may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the a modular looped out helical domain.

In some cases, modifications to a modular looped out helical domain may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a modular looped out helical domain. In some cases, modifications to a modular looped out helical domain sequences may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a modular looped out helical domain.

Modifications to a modular looped out helical domain may include substitution or addition with one or more amino acid residues. In some cases, a globular domain is capable of mediating DNA binding. In some cases, the modular looped out helical domain domain may be replaced or fused with other suitable domains capable of mediating DNA binding.

An engineered nuclease can comprise an N-terminal fragment. In some cases, an N-terminal fragment can be mutated or modified.

An N-terminal fragment of an engineered nuclease may be modified. In some cases, an N-terminal fragment may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with an N-terminal fragment of an exemplary wild-type nuclease (e.g., SEQ ID NO: 1-12 or 50-66). An N-terminal fragment may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with an N-terminal fragment of an exemplary wild-type nuclease (e.g., SEQ ID NO: 1-12 or 50-66).

In some cases, modifications to an N-terminal fragment may include but are not limited to individual amino acid modifications, as described herein. In some cases, modification to an N-terminal fragment may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of an N-terminal fragment. Modifications may include modifications to at most 1, 2, 3, 4, 5, 6, 7, 8, 9, 10, 11, 12, 13, 14, 15, 16, 17, 18, 19, 20 or more amino acids of an N-terminal fragment. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an N-terminal fragment. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an N-terminal fragment.

In some cases, modifications to an N-terminal fragment may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an N-terminal fragment. In some cases, modifications to an N-terminal fragment may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of an N-terminal fragment.

In some cases, modifications to an N-terminal fragment may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease an N-terminal fragment. In some cases, modifications to an N-terminal fragment sequence may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease an N-terminal fragment.

A middle fragment of an engineered nuclease may be modified. In some cases, a middle fragment may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a middle fragment of an exemplary wild-type nuclease (e.g., SEQ ID NO: 1-12 or 50-66). A middle fragment may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a middle fragment of an exemplary wild-type nuclease (e.g., SEQ ID NO: 1-12 or 50-66).

In some cases, modifications to a middle fragment may include but are not limited to individual amino acid modifications, as described herein. In some cases, modification to a middle fragment may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of a middle fragment. Modifications may include modifications to at most 1, 2, 3, 4, 5, 6, 7, 8, 9, 10, 11, 12, 13, 14, 15, 16, 17, 18, 19, 20 or more amino acids of a middle fragment. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a middle fragment. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a middle fragment.

In some cases, modifications to a middle fragment may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a middle fragment. In some cases, modifications to a middle fragment may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a middle fragment.

In some cases, modifications to a middle fragment may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a middle fragment. In some cases, modifications to a middle fragment sequence may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a middle fragment.

An engineered nuclease can comprise a C-terminal fragment. In some cases, a C-terminal fragment can be mutated or modified.

A C-terminal fragment of an engineered nuclease may be modified. In some cases, a C-terminal fragment may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a C-terminal fragment of an exemplary wild-type nuclease (e.g., SEQ ID NO: 1-12 or 50-66). A C-terminal fragment may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a C-terminal fragment of an exemplary wild-type nuclease (e.g., SEQ ID NO: 1-12 or 50-66).

In some cases, modifications to a C-terminal fragment may include but are not limited to individual amino acid modifications, as described herein. In some cases, modification to a C-terminal fragment may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of a C-terminal fragment. Modifications may include modifications to at most 1, 2, 3, 4, 5, 6, 7, 8, 9, 10, 11, 12, 13, 14, 15, 16, 17, 18, 19, 20 or more amino acids of a C-terminal fragment. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a C-terminal fragment. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a C-terminal fragment.

In some cases, modifications to a C-terminal fragment may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a C-terminal fragment. In some cases, modifications to a C-terminal fragment may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a C-terminal fragment.

In some cases, modifications to a C-terminal fragment may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a C-terminal fragment. In some cases, modifications to a C-terminal fragment sequence may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a C-terminal fragment.

An engineered nuclease can comprise a polypeptide fragment and/or linker region. In some cases, a polypeptide fragment and/or linker region can be mutated or modified.

A polypeptide fragment and/or linker region of an engineered nuclease may be modified. In some cases, a polypeptide fragment and/or linker region may share at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a polypeptide fragment and/or linker region of an exemplary wild-type nuclease (e.g., SEQ ID NO: 1-12 or 50-66). A polypeptide fragment and/or linker region may share at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% amino acid identity with a polypeptide fragment and/or linker region of an exemplary wild-type nuclease (e.g., SEQ ID NO: 1-12 or 50-66).

In some cases, modifications to a polypeptide fragment and/or linker region may include but are not limited to individual amino acid modifications, as described herein. In some cases, modification to a polypeptide fragment and/or linker region may include but are not limited to insertions, deletions or substitution of individual amino acids, or polypeptides, such as other protein elements (e.g domains, structural motifs, proteins).

Modifications may include modifications to at least 1, 2, 3, 4, 5, 6, 7, 8, 9, 10, 11, 12, 13, 14, 15, 16, 17, 18, 19, 20 or more amino acids of a polypeptide fragment and/or linker region. Modifications may include modifications to at most 1, 2, 3, 4, 5, 6, 7, 8, 9, 10, 11, 12, 13, 14, 15, 16, 17, 18, 19, 20 or more amino acids of a polypeptide fragment and/or linker region. Modifications may also include at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a polypeptide fragment and/or linker region. Modifications may also include at most 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a polypeptide fragment and/or linker region.

In some cases, modifications to a polypeptide fragment and/or linker region may include deletion of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a polypeptide fragment and/or linker region. In some cases, modifications to a polypeptide fragment and/or linker region may include deletion of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a polypeptide fragment and/or linker region.

In some cases, modifications to a polypeptide fragment and/or linker region may include addition or substitution of at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a polypeptide fragment and/or linker region. In some cases, modifications to a polypeptide fragment and/or linker region sequence may include addition or substitution of at most 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% of a homologous nuclease a polypeptide fragment and/or linker region.

### Guide Nucleic Acid

In general, a “guide sequence” is any polynucleotide sequence having sufficient complementarity with a target polynucleotide sequence to hybridize with the target sequence and direct sequence-specific binding of an engineered nuclease complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10-30 nucleotides long.

In general, a “scaffold sequence” includes any sequence that has sufficient sequence to promote formation of an engineered nuclease complex, wherein the engineered nuclease complex comprises an engineered nuclease and a guide nucleic acid comprising a scaffold sequence and a guide sequence. Sufficient sequence within the scaffold sequence to promote formation of an engineered nuclease complex may include a degree of complementarity along the length of two sequence regions within the scaffold sequence, such as two sequence regions involved in forming a secondary structure. In some cases, the two sequence regions are comprised or encoded on the same polynucleotide. In some cases, the two sequence regions are comprised or encoded on separate polynucleotides. Optimal alignment may be determined by any suitable alignment algorithm, and may further account for secondary structures, such as self-complementarity within either the two sequence regions. In some embodiments, the degree of complementarity between the two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions is about or more than about 5, 6, 7, 8, 9, 10, 11, 12, 13, 14, 15, 16, 17, 18, 19, 20, 25, 30, 40, 50, or more nucleotides in length.

In aspects of the invention the terms “guide nucleic acid” refers to a polynucleotide comprising 1) a guide sequence capable of hybridizing to a target sequence and 2) a scaffold sequence capable of interacting with an engineered nuclease as described herein. A guide nucleic acid together with an engineered nuclease forms an engineered nuclease complex which is capable of binding to a target sequence within a target polynucleotide, as determined by the guide sequence of the guide nucleic acid.

The ability of a guide sequence to direct sequence-specific binding of an engineered nuclease complex to a target sequence may be assessed by any suitable assay. For example, the components of a engineered nuclease system sufficient to form a engineered nuclease complex, including the guide sequence to be tested, may be provided to a host cell having the corresponding target sequence, such as by transfection with vectors encoding the components of the engineered nuclease system, followed by an assessment of preferential cleavage within the target sequence, such as by Surveyor assay as described herein. Similarly, cleavage of a target polynucleotide sequence may be evaluated in a test tube by providing the target sequence, components of a engineered nuclease complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence may be selected to target any target sequence. In some embodiments, the target sequence is a sequence within a genome of a cell. Exemplary target sequences include those that are unique in the target genome.

In general, a guide sequence is any polynucleotide sequence having sufficient complementarity with a target polynucleotide sequence to hybridize with the target sequence and direct sequence-specific binding of an engineered nuclease complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of an engineered nuclease complex to a target sequence may be assessed by any suitable assay. For example, the components of a engineered nuclease system sufficient to form a engineered nuclease complex, including the guide sequence to be tested, may be provided to a host cell having the corresponding target sequence, such as by transfection with vectors encoding the components of the engineered nuclease sequence, followed by an assessment of preferential cleavage within the target sequence, such as by Surveyor assay as described herein. Similarly, cleavage of a target sequence may be evaluated in a test tube by providing the target sequence, components of an engineered nuclease complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, a guide sequence is selected to reduce the degree secondary structure within the guide nucleic acid. In some embodiments, about or less than about 75%, 50%, 40%, 30%, 25%, 20%, 15%, 10%, 5%, 1%, or fewer of the nucleotides of the guide nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008. Cell 106(1): 23-24; and PA Carr and GM Church, 2009, Nature Biotechnology 27(12): 1151-62). A method of optimizing the guide nucleic acids of a Cas9 ortholog comprises breaking up polyU tracts in the guide RNA. PolyU tracts that may be broken up may comprise a series of 4, 5, 6, 7, 8, 9 or 10 Us.

In general, a scaffold sequence includes any sequence that has sufficient sequence to promote formation of an engineered nuclease complex at a target sequence, wherein the engineered nuclease complex comprises an engineered nucleic acid-guided nuclease and a guide nucleic acid comprising a scaffold sequence and a guide sequence. Sufficient sequence within the scaffold sequence to promote formation of an engineered nuclease complex may include a degree of complementarity along the length of two sequence regions within the scaffold sequence, such as two sequence regions involved in forming a secondary structure. In some cases, the two sequence regions are comprised or encoded on the same polynucleotide. In some cases, the two sequence regions are comprised or encoded on separate polynucleotides. Optimal alignment may be determined by any suitable alignment algorithm, and may further account for secondary structures, such as self-complementarity within either the two sequence regions. In some embodiments, the degree of complementarity between the two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions is about or more than about 5, 6, 7, 8, 9, 10, 11, 12, 13, 14, 15, 16, 17, 18, 19, 20, 25, 30, 40, 50, or more nucleotides in length. In some embodiments, the two sequence regions are contained within a single transcript, such that hybridization between the two produces a transcript having a secondary structure, such as a hairpin. In an embodiment of the invention, the transcript or transcribed polynucleotide sequence has at least two or more hairpins. In some embodiments, the transcript has two, three, four or five hairpins. In a further embodiment of the invention, the transcript has at most five hairpins.

### Polynucleic Acids and Vectors

In one aspect, the invention provides for vectors that are used in the engineering and optimization of nucleic acid-guided nuclease systems.

As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids. Further discussion of vectors is provided herein.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the .beta.-actin promoter, the phosphoglycerol kinase (PGK) promoter, and the EF1.alpha. promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit .beta.-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). With regard to regulatory sequences, mention is made of U.S. patent application Ser. No. 10/491,026, the contents of which are incorporated by reference herein in their entirety. With regards to promoters, mention is made of PCT publication WO 2011/028929 and U.S. application Ser. No. 12/511,940, the contents of which are incorporated by reference herein in their entirety.

Vectors can be designed for expression of engineered nuclease transcripts and/or guide nucleic acids (e.g. nucleic acid transcripts, proteins, enzymes, guide RNAs) in prokaryotic or eukaryotic cells. For example, engineered nuclease transcripts and/or guide nucleic acids can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerevisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the .alpha.-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments of the invention may relate to the use of viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety.

In some embodiments, a regulatory element is operably linked to one or more elements of an engineered nuclease system so as to drive expression of the one or more elements of the engineered nuclease system. In general, “engineered nuclease system” refers collectively to transcripts and other elements involved in the expression of or directing the activity of an engineered nuclease as disclosed herein, including sequences encoding an engineered nucleic acid-guided nuclease gene and a guide nucleic acid. A guide nucleic acid can comprise 1) a guide sequence capable of hybridizing to a target sequence, 2) a scaffold sequence comprising a protein binding sequence capable of interaction with an engineered nuclease as disclosed herein. In some embodiments, one or more elements of an engineered nuclease system is derived from a Type I, Type II, Type III, Type IV, Type V, or Type VI CRISPR system. In some embodiments, one or more elements of a CRISPR system is derived from one or more organisms comprising an endogenous CRISPR system, such as Eubacterium sp. XS5, Eubacterium rectale, or Succinivibrio dextrinosolvens. In general, an engineered nuclease system as disclosed herein is characterized by elements that promote the formation of a engineered nuclease complex at the site of a target sequence, wherein the engineered nuclease complex comprises an engineered nucleic acid-guided nuclease and a guide nucleic acid.

In the context of formation of a engineered nuclease complex, “target sequence” refers to a sequence to which a guide sequence is designed to have complementarity, where hybridization between a target sequence and a guide sequence promotes the formation of a engineered nuclease complex. A target sequence may comprise any polynucleotide, such as DNA or RNA polynucleotides. In some embodiments, a target sequence is located in the nucleus or cytoplasm of a cell.

Typically, formation of an engineered nuclease complex comprising a guide nucleic acid hybridized to a target sequence and complexed with one or more engineered nucleases as disclosed herein results in cleavage of one or both strands in or near (e.g. within 1, 2, 3, 4, 5, 6, 7, 8, 9, 10, 20, 50, or more base pairs from) the target sequence. In some embodiments, one or more vectors driving expression of one or more elements of an engineered nuclease system are introduced into a host cell such that expression of the elements of the engineered nuclease system direct formation of a engineered nuclease complex at one or more target sites. For example, an engineered nucleic acid-guided nuclease, and a guide nucleic acid could each be operably linked to separate regulatory elements on separate vectors. Alternatively, two or more of the elements expressed from the same or different regulatory elements, may be combined in a single vector, with one or more additional vectors providing any components of the engineered nuclease system not included in the first vector. Engineered nuclease system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding an engineered nuclease and one or more guide nucleic acids. In some embodiments, n engineered nuclease and one or more guide nucleic acids are operably linked to and expressed from the same promoter.

In some embodiments, a vector comprises one or more insertion sites, such as a restriction endonuclease recognition sequence (also referred to as a “cloning site”). In some embodiments, an insertion site can be used to incorporate a synthesized polynucleic acid comprising all or a portion of a guide nucleic acid. In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a scaffold sequence, and optionally downstream of a regulatory element operably linked to the scaffold sequence, such that following insertion of a guide sequence into the insertion site and upon expression the guide sequence directs sequence-specific binding of an engineered nuclease complex to a target sequence in a cell, such as a eukaryotic or prokaryotic cell. In some embodiments, a vector comprises two or more insertion sites, each insertion site being located between two scaffold sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target nuclease activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding an engineered nuclease as disclosed herein. An engineered nuclease can be a nucleic acid-guided nuclease. An engineered nuclease can be a chimeric nuclease comprising two or more fragments, each from a different nucleic acid-guided nuclease, such as nucleic acid-guided nucleases from different organisms.

In some embodiments, an enzyme coding sequence encoding an engineered nuclease is codon optimized for expression in particular cells, such as prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammal including non-human primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ (visited Jul. 9, 2002), and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

In some embodiments, a vector encodes an engineered nuclease comprising one or more nuclear localization sequences (NLSs), such as about or more than about 1, 2, 3, 4, 5, 6, 7, 8, 9, 10, or more NLSs. In some embodiments, the engineered nuclease comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the engineered nuclease comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO:34); the NLS from nucleoplasmin (e.g. the nucleoplasmin bipartite NLS with the sequence KRPAATKKAGQAKKKK (SEQ ID NO:35)); the c-myc NLS having the amino acid sequence PAAKRVKLD (SEQ ID NO:36) or RQRRNELKRSP (SEQ ID NO:37); the hRNPA1 M9 NLS having the sequence NQSSNFGPMKGGNFGGRSSGPYGGGGQYFAKPRNQGGY (SEQ ID NO:38); the sequence RMRIZFKNKGKDTAELRRRRVEVSVELRKAKKDEQILKRRNV (SEQ ID NO:39) of the IBB domain from importin-alpha; the sequences VSRKRPRP (SEQ ID NO:40) and PPKKARED (SEQ ID NO:41) of the myoma T protein; the sequence PQPKKKPL (SEQ ID NO:42) of human p53; the sequence SALIKKKKKMAP (SEQ ID NO:43) of mouse c-abl IV; the sequences DRLRR (SEQ ID NO:44) and PKQKKRK (SEQ ID NO:45) of the influenza virus NS1; the sequence RKLKKKIKKL (SEQ ID NO:46) of the Hepatitis virus delta antigen; the sequence REKKKFLKRR (SEQ ID NO:47) of the mouse Mxl protein; the sequence KRKGDEVDGVDEVAKKKSKK (SEQ ID NO:48) of the human poly(ADP-ribose) polymerase; and the sequence RKCLQAGMNLEARKTKK (SEQ ID NO:49) of the steroid hormone receptors (human) glucocorticoid.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the engineered nuclease, the particular NLS(s) used, or a combination of these factors. Detection of accumulation in the nucleus may be performed by any suitable technique. For example, a detectable marker may be fused to the engineered nuclease, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of the engineered nuclease complex formation (e.g. assay for DNA cleavage or mutation at the target sequence, or assay for altered gene expression activity affected by engineered nuclease complex formation and/or engineered nuclease activity), as compared to a control not exposed to the engineered nuclease or complex, or exposed to a engineered nuclease lacking the one or more NLSs.

### Delivery

An engineered nuclease and corresponding guide nucleic acid can be delivered either as DNA or RNA. Delivery of an engineered nuclease and guide nucleic acid both as RNA (normal or containing base or backbone modifications) molecules can be used to reduce the amount of time that the engineered nuclease persist in the cell. This may reduce the level of off-target cleavage activity in the target cell. Since delivery of an engineered nuclease as mRNA takes time to be translated into protein, it might be advantageous to deliver the guide nucleic acid several hours following the delivery of an engineered nuclease mRNA, to maximize the level of guide nucleic acid available for interaction with the engineered nuclease protein.

In situations where guide nucleic acid amount is limiting, it may be desirable to introduce an engineered nuclease as mRNA and guide nucleic acid in the form of a DNA expression cassette with a promoter driving the expression of the guide nucleic acid. This way the amount of guide nucleic acid available will be amplified via transcription.

Guide nucleic acid in the form of RNA or encoded on a DNA expression cassette can be introduced into a host cell comprising an engineered nuclease encoded on a vector or chromosome.

Methods and compositions disclosed herein may comprise more than one guide nucleic acid, wherein each guide nucleic acid has a different guide sequence, thereby targeting a different target sequence. In such cases, multiple guide nucleic acids can be using in multiplexing, wherein multiple targets are targeted simultaneously. Additionally or alternatively, the multiple guide nucleic acids are introduced into a population of cells, such that each cell in a population received a different or random guide nucleic acid, thereby targeting multiple different target sequences across a population of cells. In such cases, the collection of subsequently altered cells can be referred to as a library.

Methods and compositions disclosed herein may comprise multiple different engineered nucleases, each with one or more different corresponding guide nucleic acids, thereby allowing targeting of different target sequences by different engineered nucleases. In some such cases, each engineered nuclease can correspond to a distinct plurality of guide nucleic acids, allowing two or more non overlapping, partially overlapping, or completely overlapping multiplexing events.

A variety of delivery systems can be used to introduce an engineered nuclease (DNA or RNA) and guide nucleic acid (DNA or RNA) into a host cell. These include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires (Shalek et al., Nano Letters, 2012), exosomes. Molecular trojan horses liposomes (Pardridge et al., Cold Spring Harb Protoc; 2010; doi:10.1101/pdb.prot5407) may be used to deliver an engineered nuclease and guide nuclease across the blood brain barrier.

In some embodiments, a recombination template is also provided. A recombination template may be a component of another vector as described herein, contained in a separate vector, or provided as a separate polynucleotide, such as an oligonucleotide, linear polynucleotide, or synthetic polynucleotide. In some embodiments, a recombination template is designed to serve as a template in homologous recombination, such as within or near a target sequence nicked or cleaved by an engineered nuclease as a part of a complex as disclosed herein. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In some aspects, the invention provides methods comprising delivering one or more polynucleotides, such as or one or more vectors or linear polynucleotides as described herein, one or more transcripts thereof, and/or one or proteins transcribed therefrom, to a host cell. In some aspects, the invention further provides cells produced by such methods, and organisms comprising or produced from such cells. In some embodiments, an engineered nuclease in combination with (and optionally complexed with) a guide nucleic acid is delivered to a cell. Conventional viral and non-viral based gene transfer methods can be used to introduce nucleic acids in cells, such as prokaryotic cells, eukaryotic cells, mammalian cells, or target tissues. Such methods can be used to administer nucleic acids encoding components of an engineered nucleic acid-guided nuclease system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon. TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

In some embodiments, a host cell is transiently or non-transiently transfected with one or more vectors, linear polynucleotides, polypeptides, nucleic acid-protein complexes, or any combination thereof as described herein. In some embodiments, a cell in transfected in vitro, in culture, or ex vivo. In some embodiments, a cell is transfected as it naturally occurs in a subject. In some embodiments, a cell that is transfected is taken from a subject. In some embodiments, the cell is derived from cells taken from a subject, such as a cell line.

In some embodiments, a cell transfected with one or more vectors, linear polynucleotides, polypeptides, nucleic acid-protein complexes, or any combination thereof as described herein is used to establish a new cell line comprising one or more transfection-derived sequences. In some embodiments, a cell transiently transfected with the components of an engineered nucleic acid-guided nuclease system as described herein (such as by transient transfection of one or more vectors, or transfection with RNA), and modified through the activity of an engineered nuclease complex, is used to establish a new cell line comprising cells containing the modification but lacking any other exogenous sequence.

In some embodiments, one or more vectors described herein are used to produce a non-human transgenic cell, organism, animal, or plant. In some embodiments, the transgenic animal is a mammal, such as a mouse, rat, or rabbit. Methods for producing transgenic cells, organisms, plants, and animals are known in the art, and generally begin with a method of cell transformation or transfection, such as described herein.

### Engineered Nuclease Activity and Usage

In some embodiments, the engineered nuclease has DNA cleavage activity or RNA cleavage activity. In some embodiments, the engineered nuclease directs cleavage of one or both strands at the location of a target sequence, such as within the target sequence and/or within the complement of the target sequence. In some embodiments, the engineered nuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, an engineered nuclease may form a component of an inducible system. The inducible nature of the system would allow for spatiotemporal control of gene editing or gene expression using a form of energy. The form of energy may include but is not limited to electromagnetic radiation, sound energy, chemical energy, light energy, and thermal energy. Examples of inducible system include tetracycline inducible promoters (Tet-On or Tet-Off), small molecule two-hybrid transcription activations systems (FKBP, ABA, etc), or light inducible systems (Phytochrome, LOV domains, or cryptochorome). In one embodiment, the engineered nuclease may be a part of a Light Inducible Transcriptional Effector (LITE) to direct changes in transcriptional activity in a sequence-specific manner. The components of a light may include an engineered nuclease, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, which is hereby incorporated by reference in its entirety.

In some aspects, the invention provides for methods of modifying a target polynucleotide in a prokaryotic or eukaryotic cell, which may be in vivo, ex vivo, or in vitro. In some embodiments, the method comprises sampling a cell or population of cells such as prokaryotic cells, or those from a human or non-human animal or plant (including micro-algae), and modifying the cell or cells. Culturing may occur at any stage in vitro or ex vivo. The cell or cells may even be re-introduced into the host, such as a non-human animal or plant (including micro-algae). For re-introduced cells it is particularly preferred that the cells are stem cells.

In some embodiments, the method comprises allowing an engineered nuclease complex to bind to the target polynucleotide to effect cleavage of said target polynucleotide thereby modifying the target polynucleotide, wherein the engineered nuclease complex comprises an engineered nuclease complexed with a guide nucleic acid wherein the guide sequence of the guide nucleic acid is hybridized to a target sequence within said target polynucleotide.

In some aspects, the invention provides a method of modifying expression of a polynucleotide in a prokaryotic or eukaryotic cell. In some embodiments, the method comprises allowing an engineered nuclease complex to bind to the polynucleotide such that said binding results in increased or decreased expression of said polynucleotide; wherein the engineered nuclease complex comprises an engineered nuclease complexed with a guide nucleic acid, and wherein the guide sequence of the guide nucleic acid is hybridized to a target sequence within said polynucleotide. Similar considerations apply as above for methods of modifying a target polynucleotide. In fact, these sampling, culturing and re-introduction options apply across the aspects of the present invention.

In some aspects, the invention provides kits containing any one or more of the elements disclosed in the above methods and compositions. Elements may provide individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

In some aspects, the invention provides methods for using one or more elements of an engineered nucleic acid-guided nuclease system. An engineered nuclease complex of the invention provides an effective means for modifying a target sequence within a target polynucleotide. An engineered nuclease complex of the invention has a wide variety of utility including modifying (e.g., deleting, inserting, translocating, inactivating, activating) a target sequence in a multiplicity of cell types. As such an engineered nuclease complex of the invention has a broad spectrum of applications in, e.g., biochemical pathway optimization, genome-wide studies, genome engineering, gene therapy, drug screening, disease diagnosis, and prognosis. An exemplary engineered nuclease complex comprises a engineered nuclease as disclosed herein complexed with a guide nucleic acid, wherein the guide sequence of the guide nucleic acid is hybridized to a target sequence within the target polynucleotide. A guide nucleic acid can comprise a guide sequence linked to a scaffold sequence. A scaffold sequence can comprise two sequence regions with a degree of complementarity such that together they form a secondary structure. In some cases, the two sequence regions are comprised or encoded on the same polynucleotide. In some cases, the two sequence regions are comprised or encoded on separate polynucleotides.

In some embodiments, this invention provides methods of cleaving a target polynucleotide. The method comprises modifying a target polynucleotide using an engineered nuclease complex that binds to a target sequence within a target polynucleotide and effect cleavage of said target polynucleotide. Typically, the engineered nuclease complex of the invention, when introduced into a cell, creates a break (e.g., a single or a double strand break) in the genome sequence. For example, the method can be used to cleave a disease gene in a cell, or to replace a wildtype sequence with a modified sequence.

In some embodiments, when the target sequence is double stranded DNA, binding of the engineered nuclease to the target sequence can induce separation of the DNA strands. In such cases, one nuclease domain can bind and cleave one strand, such as the one containing the target sequence. A second nuclease domain can bind and cleave the complementary sequence of the target sequence, which is the non-target strand.

In some embodiments, an engineered nuclease comprises one or more domain that is capable of mediating DNA binding. In some examples, such the domain is a modular looped out helical domain capable of mediating DNA binding.

In some embodiments, an engineered nuclease comprises one or more domain that is capable of interacting with a displaced DNA sequence complementary to the target DNA sequence. In some examples, this domain is a globular domain. In some examples, a globular domain capable of interacting with a displaced DNA sequence complementary to the target DNA sequence.

In some embodiments, an engineered nuclease comprises one or more domains capable of cleaving a target sequence. In some examples, such a domain is a nuclease domain. In some examples, such a domain is a RuvC domain, RuvC-like domain, HNH domain, HNH-like domain, Zinc Finger domain, Zinc Finger-like domain.

In some embodiments, one or more of a RuvC domain, RuvC-like domain, HNH domain, HNH-like domain, Zinc Finger domain, Zinc Finger-like domain, a globular domain, is a modular looped out helical domain, or any combination thereof is comprised within an N-terminal fragment, domain, or sequence.

In some embodiments, one or more of a RuvC domain, RuvC-like domain, HNH domain, HNH-like domain, Zinc Finger domain, Zinc Finger-like domain, a globular domain, is a modular looped out helical domain, or any combination thereof is comprised within a middle fragment, domain, or sequence.

In some embodiments, one or more of a RuvC domain, RuvC-like domain, HNH domain, HNH-like domain, Zinc Finger domain, Zinc Finger-like domain, a globular domain, is a modular looped out helical domain, or any combination thereof is comprised within a C-terminal fragment, domain, or sequence.

The break created by the engineered nuclease complex can be repaired by a repair processes such as the error prone non-homologous end joining (NHEJ) pathway, the high fidelity homology-directed repair (HDR), or by recombination pathways. During these repair processes, an exogenous polynucleotide template can be introduced into the genome sequence. In some methods, the HDR or recombination process is used to modify a genome sequence. For example, an exogenous polynucleotide template comprising a sequence to be integrated flanked by an upstream sequence and a downstream sequence is introduced into a cell. The upstream and downstream sequences share sequence similarity with either side of the site of integration in the chromosome, target vector, or target polynucleotide.

Where desired, a donor template polynucleotide can be DNA, e.g., a DNA plasmid, a bacterial artificial chromosome (BAC), a yeast artificial chromosome (YAC), a viral vector, a linear piece of DNA, a PCR fragment, oligonucleotide, synthetic polynucleotide, a naked nucleic acid, or a nucleic acid complexed with a delivery vehicle such as a liposome or poloxamer.

An exogenous template polynucleotide can comprise a sequence to be integrated (e.g., a mutated gene). A sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function. Sequence to be integrated may be a mutated or variant of an endogenous wildtype sequence. Alternatively, sequence to be integrated may be a wildtype version of an endogenous mutated sequence. Additionally or alternatively, sequenced to be integrated may be a variant or mutated form of an endogenous mutated or variant sequence. In any of these examples, the exogenous template may also comprise a screenable marker, a selectable marker, a nucleic acid barcode, any other targeting or tracking mechanism, or any combination thereof.

Upstream and downstream sequences in the exogenous template polynucleotide are selected to promote recombination between the target polynucleotide of interest and the donor template polynucleotide. The upstream sequence is a nucleic acid sequence that can share sequence similarity with the sequence upstream of the targeted site for integration. Similarly, the downstream sequence is a nucleic acid sequence that can share sequence similarity with the sequence downstream of the targeted site of integration. The upstream and downstream sequences in the exogenous template polynucleotide can have 75%, 80%, 85%, 90%, 95%, or 100% sequence identity with the targeted polynucleotide. Preferably, the upstream and downstream sequences in the exogenous template polynucleotide have about 95%, 96%, 97%, 98%, 99%, or 100% sequence identity with the targeted polynucleotide. In some methods, the upstream and downstream sequences in the exogenous template polynucleotide have about 99% or 100% sequence identity with the targeted polynucleotide.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence has about 200 bp to about 2000 bp, about 600 bp to about 1000 bp, or more particularly about 700 bp to about 1000 bp.

In some methods, the exogenous template polynucleotide may further comprise a marker. Such a marker may make it easy to screen for targeted integrations. Examples of suitable markers include restriction sites, fluorescent proteins, or selectable markers. The exogenous polynucleotide template of the invention can be constructed using recombinant techniques (see, for example, Sambrook et al., 2001 and Ausubel et al., 1996).

In an exemplary method for modifying a target polynucleotide by integrating an exogenous template polynucleotide, a double stranded break is introduced into the genome sequence by an engineered nuclease complex, the break is repaired via homologous recombination using an exogenous template polynucleotide such that the template is integrated into the target polynucleotide. The presence of a double-stranded break facilitates integration of the template.

In some embodiments, this invention provides methods of modifying expression of a polynucleotide in a cell. The method comprises increasing or decreasing expression of a target polynucleotide by using an engineered nuclease complex that binds to the target polynucleotide.

In some methods, a target polynucleotide can be inactivated to effect the modification of the expression in a cell. For example, upon the binding of an engineered nuclease complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein is not produced.

In some methods, a control sequence can be inactivated such that it no longer functions as a control sequence. As used herein. “control sequence” refers to any nucleic acid sequence that effects the transcription, translation, or accessibility of a nucleic acid sequence. Examples of a control sequence include, a promoter, a transcription terminator, and an enhancer are control sequences.

An inactivated target sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). In some methods, the inactivation of a target sequence results in “knockout” of the target sequence.

An altered expression of one or more target polynucleotides associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product.

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the accompanying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency, for instance as described herein. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive in Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, .beta.-galactosidase, urease, alkaline phosphatase or peroxidase, avidin/biotin complex.

Detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent:protein complex so formed. In one aspect of this embodiment, the agent that specifically binds a protein associated with a signaling biochemical pathway is an antibody, preferably a monoclonal antibody.

The reaction can be performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent:polypeptide complex. However, the label is typically designed to be accessible to an antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Anti-phosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2.alpha.). Alternatively, these antibodies can be generated using conventional polyclonal or monoclonal antibody technologies by immunizing a host animal or an antibody-producing cell with a target protein that exhibits the desired post-translational modification.

In practicing the subject method, it may be desirable to discern the expression pattern of an protein associated with a signaling biochemical pathway in different bodily tissue, in different cell types, and/or in different subcellular structures. These studies can be performed with the use of tissue-specific, cell-specific or subcellular structure specific antibodies capable of binding to protein markers that are preferentially expressed in certain tissues, cell types, or subcellular structures.

An altered expression of a gene associated with a signaling biochemical pathway can also be determined by examining a change in activity of the gene product relative to a control cell. The assay for an agent-induced change in the activity of a protein associated with a signaling biochemical pathway will dependent on the biological activity and/or the signal transduction pathway that is under investigation. For example, where the protein is a kinase, a change in its ability to phosphorylate the downstream substrate(s) can be determined by a variety of assays known in the art. Representative assays include but are not limited to immunoblotting and immunoprecipitation with antibodies such as anti-phosphotyrosine antibodies that recognize phosphorylated proteins. In addition, kinase activity can be detected by high throughput chemiluminescent assays such as AlphaScreen™ (available from Perkin Elmer) and eTag™ assay (Chan-Hui, et al. (2003) Clinical Immunology 111: 162-174).

Where the protein associated with a signaling biochemical pathway is part of a signaling cascade leading to a fluctuation of intracellular pH condition, pH sensitive molecules such as fluorescent pH dyes can be used as the reporter molecules. In another example where the protein associated with a signaling biochemical pathway is an ion channel, fluctuations in membrane potential and/or intracellular ion concentration can be monitored. A number of commercial kits and high-throughput devices are particularly suited for a rapid and robust screening for modulators of ion channels. Representative instruments include FLIPR™ (Molecular Devices, Inc.) and VIPR (Aurora Biosciences). These instruments are capable of detecting reactions in over 1000 sample wells of a microplate simultaneously, and providing real-time measurement and functional data within a second or even a minisecond.

In practicing any of the methods disclosed herein, a suitable vector can be introduced to a cell, tissue, organism, or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

A target polynucleotide of an engineered nuclease complex can be any polynucleotide endogenous or exogenous to the host cell. For example, the target polynucleotide can be a polynucleotide residing in the nucleus of the eukaryotic cell, the genome of a prokaryotic cell, or an extrachromosomal vector of a host cell. The target polynucleotide can be a sequence coding a gene product (e.g., a protein) or a non-coding sequence (e.g., a regulatory polynucleotide or a junk DNA).

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Embodiments of the invention also relate to methods and compositions related to knocking out genes, editing genes, altering genes, amplifying genes, and repairing particular mutations. Altering genes may also mean the epigenetic manipulation of a target sequence. This may be the chromatin state of a target sequence, such as by modification of the methylation state of the target sequence (i.e. addition or removal of methylation or methylation patterns or CpG islands), histone modification, increasing or reducing accessibility to the target sequence, or by promoting 3D folding. It will be appreciated that where reference is made to a method of modifying a cell, organism, or mammal including human or a non-human mammal or organism by manipulation of a target sequence in a genomic locus of interest, this may apply to the organism (or mammal) as a whole or just a single cell or population of cells from that organism (if the organism is multicellular). In the case of humans, for instance, Applicants envisage, inter alia, a single cell or a population of cells and these may preferably be modified ex vivo and then re-introduced. In this case, a biopsy or other tissue or biological fluid sample may be necessary. Stem cells are also particularly preferred in this regard. But, of course, in vivo embodiments are also envisaged. And the invention is especially advantageous as to HSCs.

Other methods, uses, or suitable systems for any of the engineered nucleases disclosed herein are described in Internation Application No. PCT/US2012/033799 filed Apr. 16, 2012, International Application No. PCT/US2015/015476 filed Feb. 11, 2015, and International Application No. PCT/US2017/039146 filed Jun. 23, 2017, the contents of each of which are herein incorporated by reference in their entirety.

### Library Generation and Screening

Libraries or engineered nucleases, including chimeric nucleases and chimeric nucleic acid-guided nucleases, can be generated using any molecular methods known in the field. In some examples, chimeric nuclease libraries can be generating by combining one or more fragments or domains from a first nuclease with one or more fragments or domains from a second nuclease in order to generate a chimeric nuclease.

In some cases, a nuclease can comprise one or more fragments or domains. To generate a chimeric nuclease, any of these fragments or domains from a first nuclease can be replaced with a corresponding fragment or domain from a different second nuclease. In some cases, two fragments or domains from a first nucleic acid are replaced by corresponding fragments or domains from a different second nuclease. In some cases, three fragments or domains from a first nucleic acid are replaced by corresponding fragments or domains from a different second nuclease. In some cases, four fragments or domains from a first nucleic acid are replaced by corresponding fragments or domains from a different second nuclease.

In some cases, a nuclease can comprise one or more fragments or domains. To generate a chimeric nuclease, any of these fragments or domains from a first nuclease can be replaced with a corresponding fragment or domain from two or more different nucleases. In some cases, two fragments or domains from a first nucleic acid are replaced by corresponding fragments or domains from two or more different nucleases. In some cases, three fragments or domains from a first nucleic acid are replaced by corresponding fragments or domains from two or more different nucleases. In some cases, four fragments or domains from a first nucleic acid are replaced by corresponding fragments or domains from two or more different nucleases.

In some cases, a nuclease can comprise one or more fragments or domains. To generate a chimeric nuclease, any of these fragments or domains from a first nuclease can be replaced with a corresponding fragment or domain from three or more different nucleases. In some cases, two fragments or domains from a first nucleic acid are replaced by corresponding fragments or domains from three or more different nucleases. In some cases, three fragments or domains from a first nucleic acid are replaced by corresponding fragments or domains from three or more different nucleases. In some cases, four fragments or domains from a first nucleic acid are replaced by corresponding fragments or domains from three or more different nucleases.

In some cases, a nuclease can comprise one or more fragments or domains. To generate a chimeric nuclease, any of these fragments or domains from a first nuclease can be replaced with a corresponding fragment or domain from four or more different nucleases. In some cases, two fragments or domains from a first nucleic acid are replaced by corresponding fragments or domains from four or more different nucleases. In some cases, three fragments or domains from a first nucleic acid are replaced by corresponding fragments or domains from four or more different nucleases. In some cases, four fragments or domains from a first nucleic acid are replaced by corresponding fragments or domains from four or more different nucleases.

In any of these cases, the one or more fragments or domains can comprise a RuvC domain, RuvC-like domain, HNH domain, HNH-like domain, Zinc Finger domain, Zinc Finger-like domain, globular domain, modular looped out helical domain, N-terminal fragment, middle fragment, C-terminal fragment, or any combination thereof.

An N-terminal fragment can comprise one or more domains. Such domains can comprise a RuvC domain, RuvC-like domain, HNH domain, HNH-like domain, Zinc Finger domain, Zinc Finger-like domain, globular domain, modular looped out helical domain, linker domain, or any combination thereof.

A middle fragment can comprise one or more domains. Such domains can comprise a RuvC domain, RuvC-like domain, HNH domain, HNH-like domain, Zinc Finger domain, Zinc Finger-like domain, globular domain, modular looped out helical domain, linker domain, or any combination thereof.

A C-terminal fragment can comprise one or more domains. Such domains can comprise a RuvC domain, RuvC-like domain, HNH domain, HNH-like domain, Zinc Finger domain, Zinc Finger-like domain, globular domain, modular looped out helical domain, linker domain, or any combination thereof.

In some cases, a nuclease can comprise an N-terminal fragment, middle fragment, and C-terminal fragment. To generate a chimeric nuclease, any of these fragments, or a portion of these fragments from a first nuclease, can be replaced with a corresponding fragment or portion of the fragment from one or more different nucleases. A fragment or portion of a fragment can comprise one or more functional domains. A fragment or portion of a fragment can comprise a linker domain.

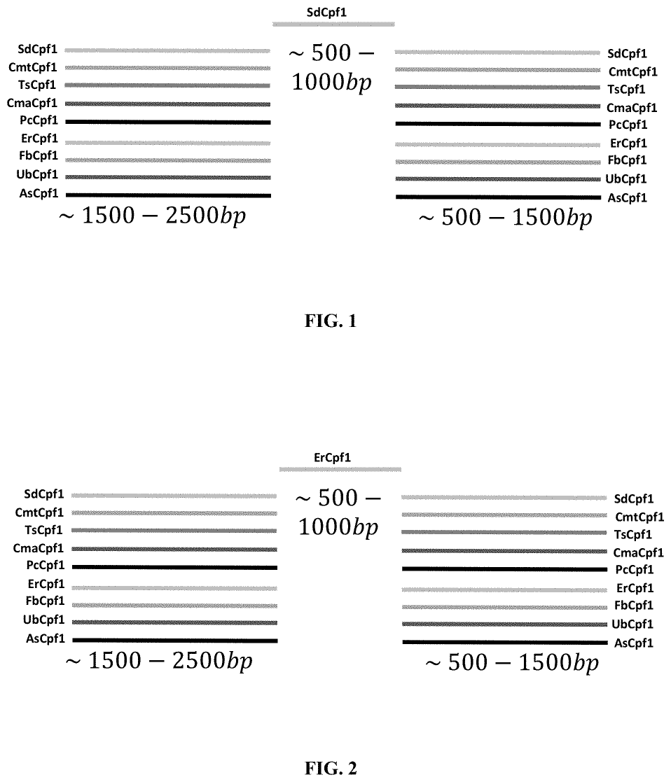

Chimeric nuclease libraries can be generated by combining nucleic acid sequences encoding one or more fragments, portion of fragments, functional domains, or linker regions. Combining these nucleic acid sequences can occur by chemical synthesis, Gibson assembly, SLIC, CPEC, PCA, ligation-free cloning, other in vitro oligo assembly techniques, traditional ligation-based cloning, or any combination thereof. The starting material for any of these generation methods can be PCR amplified fragments, synthesized oligonucleotides, or digested fragments of isolated genomic DNA. Examples of an assembly scheme are depicted in FIG. 1 and FIG. 2.

A nucleic acid sequence encoding an engineered or chimeric nuclease can be from 20 nucleotides to 5000 nucleotides in length. For example, a particular sub-segment can comprise about 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, or greater than 2500 nucleotides. It should be understood that a nucleic acid sequence to be used in a library generation can be any length, including any whole number in between the explicitly recited numbers, as well as any whole number outside the indicated range. The length of the nucleic acid sequence sub-segment used will depend on the design of the experiment, the length of the protein fragment or domain to be assembled, or any other number of factors that change or guide experimental design.

In some cases, an N-terminal nucleic acid sequence is about 500 to about 2500 nucleotides in length. For example, the N-terminal nucleic acid sequence can be about 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, or 2500 nucleotides in length. In some cases, the N-terminal nucleic acid sequence is greater the 500 nucleotides in length. In some cases, the N-terminal nucleic acid sequence is less than 500 nucleotides in length. In some cases, the N-terminal nucleic acid sequence is greater the 2500 nucleotides in length. In some cases, the N-terminal nucleic acid sequence is less than 2500 nucleotides in length.

In some cases, a middle nucleic acid sequence is about 500 to about 2500 nucleotides in length. For example, the middle nucleic acid sequence can be about 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, or 2500 nucleotides in length. In some cases, the middle nucleic acid sequence is greater the 500 nucleotides in length. In some cases, the middle nucleic acid sequence is less than 500 nucleotides in length. In some cases, the middle nucleic acid sequence is greater the 2500 nucleotides in length. In some cases, the middle nucleic acid sequence is less than 2500 nucleotides in length.

In some cases, an C-terminal nucleic acid sequence is about 500 to about 2500 nucleotides in length. For example, the C-terminal nucleic acid sequence can be about 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, or 2500 nucleotides in length. In some cases, the C-terminal nucleic acid sequence is greater the 500 nucleotides in length. In some cases, the C-terminal nucleic acid sequence is less than 500 nucleotides in length. In some cases, the C-terminal nucleic acid sequence is greater the 2500 nucleotides in length. In some cases, the C-terminal nucleic acid sequence is less than 2500 nucleotides in length.

Nucleic acid sub-segments can comprise can comprise flanking homology regions that share homology to the adjacent nucleic acid sub-segment to which is will be combined. In other words, two adjacent sub-segments that are to be combined, such as by a DNA assembly method, can have overlapping regions of homology to enable homologous recombination or recombineering. These overlapping homology regions can be about 5, 10, 15, 20, 25, 30, 35, 40, 45, 50, 60, 70, 80, 90, 100, 200, 300, 400, 500, 600, 700, 800, or more than 800 nucleotides in length. The length of the overlapping homology region can depend on the experimental design, method of cloning, and many other factors, so it should be recognized that any suitable overlapping homology region length is envisioned. Overlapping homology regions can be added to nucleic acid sub-segments through any method disclosed herein, including PCR, DNA synthesis, or DNA assembly.

Generated nucleic acid sequences encoding an engineered or chimeric nuclease can be cloned into a vector backbone. The vector backbone can be added during the generation of the chimeric nuclease nucleic acid generation, or the vector backbone can be added subsequent to the generation. The vector backbone can be added by any method disclosed herein or known in the art, including DNA assembly, Gibson assembly, PCR, and ligation-based cloning.

A vector backbone used in the generation of an engineered or chimeric nuclease library can be any vector disclosed herein. The vector can comprise additional elements, such as a selectable marker, promoter, terminator, or other regulatory element operable in a suitable host cell. The vector can comprise any other additional element disclosed herein, including a nucleic acid barcode or inducible expression system. In some examples, the vector may also comprise other components of a nucleic acid guided-nuclease system, such as a guide nucleic acid or donor template.

It should be recognized that there are numerous possible permutations of chimeric nucleases generated from any of the nucleases disclosed herein. Therefore, it can be advantageous to screen or select for chimeric nucleases with a desired function or property.

In some examples, functional selection may include selecting for chimeric nucleases capable of cleaving a target sequence. Selections can be design that enrich for such functional nucleases. For example, a positive selection method can require a target sequence be cleaved by the chimeric nuclease in order to escape cell death. In such cases, surviving cells are enriched for cells comprising a functional chimeric nuclease. The vector comprised within cells surviving the positive selection can be subsequently sequenced to determine the identity of the encoded chimeric nuclease. In cases where the vectors comprise a barcode, the barcode can be sequenced to identify the encoded chimeric nuclease.

Positive selectable markers can be an element that confers a selective advantage to the host cell, such as an antibiotic resistance gene. A positive selection can also be the disablement of a negative selectable marker that would otherwise eliminate or inhibit the growth of the host cell. In such cases, cells expressing function nucleases capable of cleaving the negative selectable marker will survive, but host cells expressing a non-functional nuclease will be unable to cleave the target sequence and with therefore die.

In some examples, the chimeric nuclease library comprises a library of chimeric nucleic acid-guided nucleases. In such cases, functional selection methods can further comprise delivery of a compatible guide nucleic acid, and optionally a donor template. The guide nucleic acid can be designed to target the target sequence involved in the positive selection. The optional donor template can comprise a desired mutation or stop codon involved in the positive selection.

It should be understood that negative selection experiments can also be used to identify functional nucleases. In such cases, the selection used in the experimental design will cause cell death in the cells expressing a functional nuclease. In these cases, a control population without the selective pressure is replica plates alongside the cells subjected to the selection pressure. Cells that die under the selection pressure can then be identified by picking the cells or colony from the control replica plate.

Negative selectable markers can be an element that eliminates or inhibits growth of the host cell upon selection. A negative selection can also be achieved by targeting a positive selectable marker, such as an antibiotic resistance gene. In such cases, cells expressing function nucleases capable of cleaving the positive selectable marker will die, but host cells expressing a non-functional nuclease will be unable to cleave the target sequence and will therefore survive.

It should be understood that screening methods can also be used to identify function nucleases. In such cases, the screenable marker can be targeting by the library of nucleases. The experiment can be designed to have the screenable marked, such as GFP or other fluorescent protein or marker, be turned on or off in the present of a function nuclease.

Screenable and selectable markers and genes are well known in the art. The disclosed methods envision use of any suitable selectable or screenable marker. Selection of the suitable marker can depend on the host cell and experimental goal.

### Some Definitions

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993). Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25 degrees Celsius. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15 degrees Celsius lower than the Tm. In order to require at least about 70% nucleotide complementarity of hybridized sequences, moderately-stringent washing conditions are selected to be about 15 to 30 degrees Celsius lower than the Tm. Highly permissive (very low stringency) washing conditions may be as low as 50 degrees Celsius below the Tm, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “domain” or “protein domain” refers to a part of a protein sequence that may exist and function independently of the rest of the protein chain.

As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. Sequence homologies may be generated by any of a number of computer programs known in the art, for example BLAST or FASTA, etc. A suitable computer program for carrying out such an alignment is the GCG Wisconsin Bestfit package (University of Wisconsin. U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Percent homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity.

However, these more complex methods assign “gap penalties” to each gap that occurs in the alignment so that, for the same number of identical amino acids, a sequence alignment with as few gaps as possible—reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore first requires the production of an optimal alignment, taking into consideration gap penalties. A suitable computer program for carrying out such an alignment is the GCG Wisconsin Bestfit package (Devereux et al., 1984 Nuc. Acids Research 12 p 387). Examples of other software that may perform sequence comparisons include, but are not limited to, the BLAST package (see Ausubel et al., 1999 Short Protocols in Molecular Biology, 4th Ed.—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health).

Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result.

Sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. However, it is more useful to include mutation data as well. The sets of amino acids thus derived are likely to be conserved for structural reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids.

Embodiments of the invention include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids such as basic for basic, acidic for acidic, polar for polar, etc. Non-homologous substitution may also occur i.e., from one class of residue to another or alternatively involving the inclusion of unnatural amino acids such as omithine (hereinafter referred to as Z), diaminobutyric acid omithine (hereinafter referred to as B), norleucine ornithine (hereinafter referred to as O), pyridylalanine, thienylalanine, naphthylalanine and phenylglycine.

Variant amino acid sequences may include suitable spacer groups that may be inserted between any two amino acid residues of the sequence including alkyl groups such as methyl, ethyl or propyl groups in addition to amino acid spacers such as glycine or .beta.-alanine residues. A further form of variation, which involves the presence of one or more amino acid residues in peptoid form, may be well understood by those skilled in the art. For the avoidance of doubt, “the peptoid form” is used to refer to variant amino acid residues wherein the .alpha.-carbon substituent group is on the residue's nitrogen atom rather than the .alpha.-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Engineered Nucleases

Nucleases with approximately 35% identity to SEQ ID NO: 30 or approximately 35% identity to SEQ ID NO: 31 were identified, some of which are listed in Table 1 and Table 2 respectively. Coding sequences for select orthologues were optionally codon optimized and then synthesized and assembled into an expression vector. Variant libraries are generated by separately mutating each amino acid residue using recombineering with barcoded synthetic constructs. Viable variants are assessed in a functional cleavage assay.

TABLE 1

| SEQ ID NO: | Organism |
|---|---|
| 1 | Thiomicrospira sp. XS5 |
| 2 | Eubacterium rectale |
| 50 | Succinivibrio dextrinosolvens |
| 51 | Candidatus Methanoplasma termitum |
| 52 | Candidatus Methanomethylophilus alvus |
| 53 | Porphyromonas crevioricanis |
| 54 | Flavobacterium branchiophilum |
| 55 | Lachnospiraceae bacterium COE1 |
| 56 | Prevotella brevis ATCC 19188 |
| 57 | Smithella sp. SCADC protein 1 |
| 58 | Moraxella bovoculi |
| 59 | Synergistes jonesii |
| 60 | Bacteroidetes oral taxon 274 |
| 61 | Francisella tularensis |
| 62 | Leptospira inadai serovar Lyme str. 10 |
| 30 | Acidomonococcus sp. |
| 66 | Smithella sp. SCADC protein 2 |

TABLE 2

| SEQ ID NO: | Organism |
|---|---|
| 3 | Catenibacterium sp. CAG: 290 |
| 4 | Kandleria vitulina |
| 5 | Clostridiales bacterium KA00274 |
| 6 | Lachnospiraceae bacterium 3-2 |
| 7 | Dorea longicatena |
| 8 | Coprococcus catus GD/7 |
| 9 | Enterococcus columbae DSM 7374 |
| 10 | Fructobacillus sp. EFB-N1 |
| 11 | Weissella halotolerans |
| 12 | Pediococcus acidilactici |
| 31 | Streptococcus pyogenes |
| 63 | Lactobacillus curvatus |
| 64 | Lactobacillus versmoldensis |
| 65 | Filifactor alocis ATCC 35896 |

### Example 2. Chimeric Nucleases

Chimeric nucleases are generated with fragments from Cpf1 orthologues and variants identified in Example 1. Some of the chimeric nucleases contain at least one RuvC domain and/or a Zinc finger-like domain from Eubacterium rectale or Succinivibrio dextrinosolvens. Other chimeric nucleases contain at least one RuvC domain or a Zinc finger-like domain from any nuclease listed in Table 1. Some of the chimeric nucleases contain an N-terminal fragment or a C-terminal fragment from Eubacterium rectale or Succinivibrio dextrinosolvens. Other chimeric nucleases contain an N-terminal fragment or a C-terminal fragment from any nuclease listed in Table 1. Some of the chimeric nucleases comprise a RuvC domain from first nuclease and a Zinc finger-like domain from a second nuclease, where the first and second nucleases are any two nucleases listed in Table 1. Examples of such pairs are listed in Table 3. Some of the chimeric nucleases comprise an N-terminal fragment from first nuclease and a C-terminal fragment from a second nuclease, where the first and second nucleases are any two nucleases listed in Table 1. Examples of such pairs are listed in Table 3.

In other experiments, chimeric nucleases are generated such that the middle sequence of a first nuclease is replaced with the middle sequence of a second nuclease. The resulting chimeric nuclease has a N-terminal sequence of the first nuclease, followed by the middle sequence of the second nuclease, followed by the C-terminal sequence of the first nuclease. Combinations of the first and second nucleases to be used in these chimeric nucleases are any two nucleases listed in Table 1. Examples of such pairs are listed in Table 3. In some examples, the middle sequence is from either Eubacterium rectale or Succinivibrio dextrinosolvens. The N-terminal, middle, and C-terminal sequences can be determined as described in Example 6.

In other experiments, chimeric nucleases are generated such that the middle sequence of a first nuclease is replaced with the middle sequence of a second nuclease, and the C-terminal sequence of the first nuclease is replaced by the C-terminal sequence of a third nuclease. The resulting chimeric nuclease has a N-terminal sequence of the first nuclease, followed by the middle sequence of the second nuclease, followed by the C-terminal sequence of the third nuclease. Combinations of the first, second, and third nucleases to be used in these chimeric nucleases are any three nucleases listed in Table 1. In some examples, the example pairs listed in Table 3 are combined with one other nuclease selected from Table 1. In some examples, the middle sequence is from either Eubacterium rectale or Succinivibrio dextrinosolvens.

TABLE 3

| Chimeric | | |
|---|---|---|
| protein # | First nuclease derived from | Second nuclease derived from |
| 1 | Succinivibrio dextrinosolvens | Succinivibrio dextrinosolvens |
| 2 | Succinivibrio dextrinosolvens | Eubacterium rectale |
| 3 | Succinivibrio dextrinosolvens | Thiomicrospira sp. XS5 |
| 4 | Succinivibrio dextrinosolvens | Candidatus Methanoplasma termitum |
| 5 | Succinivibrio dextrinosolvens | Candidatus Methanomethylophilus alvus |
| 6 | Succinivibrio dextrinosolvens | Porphyromonas crevioricanis |
| 7 | Succinivibrio dextrinosolvens | Flavobacterium branchiophilum |
| 8 | Succinivibrio dextrinosolvens | Lachnospiraceae bacterium COE1 |
| 9 | Succinivibrio dextrinosolvens | Prevotella brevis ATCC 19188 |
| 10 | Succinivibrio dextrinosolvens | Smithella sp. SCADC protein 1 or 2 |
| 11 | Succinivibrio dextrinosolvens | Moraxella bovoculi |
| 12 | Succinivibrio dextrinosolvens | Synergistes jonesii |
| 13 | Succinivibrio dextrinosolvens | Bacteroidetes oral taxon 274 |
| 14 | Succinivibrio dextrinosolvens | Francisella tularensis |
| 15 | Succinivibrio dextrinosolvens | Leptospira inadai serovar Lyme str. 10 |
| 16 | Succinivibrio dextrinosolvens | Acidomonococcus sp. |
| 32 | Eubacterium rectale | Eubacterium rectale |
| 33 | Eubacterium rectale | Succinivibrio dextrinosolvens |
| 34 | Eubacterium rectale | Candidatus Methanoplasma termitum |
| 35 | Eubacterium rectale | Candidatus Methanomethylophilus alvus |
| 36 | Eubacterium rectale | Porphyromonas crevioricanis |
| 37 | Eubacterium rectale | Flavobacterium branchiophilum |
| 38 | Eubacterium rectale | Lachnospiraceae bacterium COE1 |
| 39 | Eubacterium rectale | Prevotella brevis ATCC 19188 |
| 40 | Eubacterium rectale | Smithella sp. SCADC protein 1 or 2 |
| 41 | Eubacterium rectale | Moraxella bovoculi |
| 42 | Eubacterium rectale | Synergistes jonesii |
| 43 | Eubacterium rectale | Bacteroidetes oral taxon 274 |
| 44 | Eubacterium rectale | Francisella tularensis |
| 45 | Eubacterium rectale | Leptospira inadai serovar Lyme str. 10 |
| 46 | Eubacterium rectale | Acidomonococcus sp. |

### Example 3. Chimeric Nucleases

Chimeric nucleases are generated with fragments from Cas9 orthologues and variants identified in Example 1. Some of the chimeric nucleases contain at least one RuvC domain and/or a HNH domain from Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici. Other examples contain at least one RuvC domain and/or a HNH domain from any nuclease listed in table 2. Some of the chimeric nucleases contain an N-terminal fragment and/or a C-terminal fragment from Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici. Other example chimeric nucleases contain an N-terminal fragment and/or a C-terminal fragment from any nuclease listed in Table 2. Some of the chimeric nucleases comprise a RuvC domain from first nuclease and a HNH domain from a second nuclease, where the first and second nucleases are any two nucleases listed in Table 2. Some of the chimeric nucleases comprise an N-terminal fragment from first nuclease and a C-terminal fragment from a second nuclease, where the first and second nucleases are any two nucleases listed in Table 2.

In other experiments, chimeric nucleases are generated such that the middle sequence of a first nuclease is replaced with the middle sequence of a second nuclease. The resulting chimeric nuclease has an N-terminal sequence of the first nuclease, followed by the middle sequence of the second nuclease, followed by the C-terminal sequence of the first nuclease. Combinations of the first and second nucleases to be used in these chimeric nucleases are any two nucleases listed in Table 2. In some cases, at least one of the nucleases is Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici. The N-terminal, middle, and C-terminal sequences can be determined as described in Example 6.

In other experiments, chimeric nucleases are generated such that the middle sequence of a first nuclease is replaced with the middle sequence of a second nuclease, and the C-terminal sequence of the first nuclease is replaced by the C-terminal sequence of a third nuclease. The resulting chimeric nuclease has a N-terminal sequence of the first nuclease, followed by the middle sequence of the second nuclease, followed by the C-terminal sequence of the third nuclease. Combinations of the first, second, and third nucleases to be used in these chimeric nucleases are any three nucleases listed in Table 2. In some cases, at least one of the nucleases is Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, or Pediococcus acidilactici.

### Example 4. Engineered Nucleases Cloning and Functional Assay

Chimeric nucleases described in Examples 2-3 are codon optimized for expression in E. coli and are integrated into a safe site using 200 bp homology arms. Coding sequences are under the control of an arabinose inducible promoter.

Chimeric nucleases and corresponding guide nucleic acids were used in a functional cleavage assay. Initial tests are performed using an assumed protospacer adjacent motif (PAM) of TTT. Data from initial tests are used to refine PAM specificity or to determine Pam by depletion assay.

Functional cleavage assay is performed by transforming a guide nucleic acid and editing template into E. coli expressing a chimeric nuclease to be tested. Following transformation, cells are plated and, following overnight selection, editing efficiency is assessed by colorimetric colony screening and/or sequencing.

### Example 5. Genome Editing with Chimeric Nuclease

A chimeric nuclease as described in Example 4 is separately introduced into E. coli and yeast. A guide nucleic acid targeting a gene of interest, along with a repair template comprising a desired mutation, are introduced into the E. coli and yeast cells. Within the cells, the chimeric nuclease forms a complex with the guide nucleic acid and subsequently cleaves the target gene. The provided repair template is used to repair the cleaved gene by recombination, homology driven repair, or non-homologous end joining. Repaired cells are selected and confirmed to carry the desired gene mutation.

### Example 6. Construction of a First Chimeric Nuclease Library

A first chimeric nuclease library was constructed using a mixture of N-terminal, middle, and C-terminal sequences from various enzymes of the Cpf1 family. A PCR and Gibson-based assembly approach was used to construct these chimeric protein libraries. The strategy was based on the dissection of the Cpf1 proteins into three segments based on an optimized amino acid alignment. The alignment demarcates the proteins (e.g., Succinivibrio dextrinosolvens Cpf1 (“SdCpf1”, refseq AJI56734.1, SEQ ID NO: 50) and Eubacterium rectale Cpf1 (“ErCpf1”, refseq WP_055225123.1, SEQ ID NO: 2) proteins) into 3 basic units. The N-terminal portion of the protein (amino acids 1-651 of SEQ ID NO: 50 for SdCpf1 and 1-672 of SEQ ID NO: 2 for ErCpf1) demarcate the globular domains that end at the modular looped out helical domain (LHD). The LHD acts to mediate DNA binding (Dong et al. Nature. 2016 Apr. 28; 532(7600):522-6). The C-terminal portion was derived from the downstream portions of these nucleases and contains a second globular domain that is positioned to interact with the displaced non-target DNA.

Chimeric nucleases were made using N-terminal and C-terminal sequences from the following Cpf1 family enzymes: Succinivibrio dextrinosolvens (SdCpf1, SEQ ID NO: 50), Candidatus methanoplasma termitum (CmtCpf1, SEQ ID NO: 51), Thiomicrospira sp. XS5 (TsCpf1, SEQ ID NO: 1), Candidatus methanomethylophilus alvus (CmaCpf1, SEQ ID NO: 52), Porphyromonas crevioricanis (PcCpf1, SEQ ID NO: 53), Eubacterium rectale (ErCpf1, SEQ ID NO: 2), Flavobacterium branchiophilum (FbCpf1, SEQ ID NO: 54), an uncultured bacterium (UbCpf1) and Acidomonococcus sp. (AsCpf1, SEQ ID NO: 30). The middle region of the first library included sequences from SdCpf1. As shown in FIG. 1, between approximately 500 to 1500 base pairs of the middle region of SdCpf1 was assembled with flanking N-terminal and C-terminal regions of the indicated Cpf1 family members, each comprising between approximately 500 to 2500 base pairs. Corresponding sequence identifiers for the nucleic acid sequences used in the library generation are provided in Table 5.

TABLE 5

| Name | Sequences |
|---|---|
| SdCpf1 N-Terminus Sequence | SEQ ID NO: 67 |
| CmtCpf1 N-Terminus Sequence | SEQ ID NO: 68 |
| TsCpf1 N-Terminus Sequence | SEQ ID NO: 69 |
| CmaCpf1 N-Terminus Sequence | SEQ ID NO: 70 |
| PcCpf1 N-Terminus Sequence | SEQ ID NO: 71 |
| ErCpf1 N-Terminus Sequence | SEQ ID NO: 72 |
| FbCpf1 N-Terminus Sequence | SEQ ID NO: 73 |
| UbCpf1 N-Terminus Sequence | SEQ ID NO: 74 |
| AsCpf1 N-Terminus Sequence | SEQ ID NO: 75 |
| ErCpf1 Middle Sequence | SEQ ID NO: 76 |
| SdCpf1 C-Terminus Sequence | SEQ ID NO: 77 |
| CmtCpf1 C-Terminus Sequence | SEQ ID NO: 78 |
| TsCpf1 C-Terminus Sequence | SEQ ID NO: 79 |
| CmaCpf1 C-Terminus Sequence | SEQ ID NO: 80 |
| PcCpf1 C-Terminus Sequence | SEQ ID NO: 81 |
| ErCpf1 C-Terminus Sequence | SEQ ID NO: 82 |
| FbCpf1 C-Terminus Sequence | SEQ ID NO: 83 |
| UbCpf1 C-Terminus Sequence | SEQ ID NO: 84 |
| AsCpf1 C-Terminus Sequence | SEQ ID NO: 85 |

The various domains were separately PCR amplified using the Q5 polymerase from NEB (Ipswich, Mass.) according to the manufacturer's protocol. Following PCR each middle fragment amplicon was pooled with orthogonal upstream or downstream fragments in a separate Gibson reaction to create combinatorial libraries. The N-terminal sequences, the middle sequence, the C-terminus sequences, and the vector backbone were combined to a final concentration of 0.2 pmol of all the segments. Vector alone was used as control, with the amount of vector standardized to be the same as the final concentration of vector in the chimeric nuclease reactions.

The various sequence regions were assembled using Gibson Assembly@ HiFi 1-Step Kit (SGI-DNA, La Jolla, Calif.), 50° C. for 4 hours. Following assembly, the DNA vectors were transformed into E. coli 10GF′ ELITE™ Electrocompetent Cells (Lucigen, Middleton, Wis.). After recovery, 50 μl of cells were transformed with the chimeric nuclease library or the control vector, and were plated and cultured at 30° C. overnight. Next day, the plasmid library was purified from the transformed cells using a Qiagen plasmid miniprep kit.

A library coverage of >95% was estimated based on >10 fold colony counts relative to the possible library size.

### Example 7: Construction of a Second Chimeric Nuclease Library

A second library was constructed as set forth above in Example 6. The sdCPF1 middle sequence was replaced in this library by an ErCpf1. The chimeric nucleases were structured as depicted in FIG. 2. Chimeric nucleases were again made using sequences from the following Cpf1 family enzymes: Succinivibrio dextrinosolvens (SdCpf1), Candidatus Methanoplasma termitum (CmtCpf1), Thiomicrospira sp. XS5 (TsCpf1), Candidatus methanomethylophilus alvus (CmaCpf1), Porphyromonas crevioricanis (PcCpf1), Eubacterium rectale (ErCpf1), Flavobacterium branchiophilum (FbCpf1) an uncultured bacterium (UbCpf1) and Acidomonococcus sp. (AsCpf1). The middle region of the second library included sequences from ErCpf1 (SEQ ID NO: 86). Between approximately 500 to 1500 base pairs of the middle region of ErCpf1 was assembled with flanking N-terminal and C-terminal regions of the indicated Cpf1 family members, each comprising between approximately 500 to 2500 base pairs.

### Example 8: Enrichment of Functional Chimeric Nucleases

The chimeric nucleases of the first and second libraries (from Examples 6 and 7 respectively) were tested for functionality by performing functional editing using the 2-deoxygalactose (2-DOG) selections as previously described. See, e.g., WO 2016105405 A1; Warming, et al., Nucleic Acids Res. 33, e36 (2005); Herring, C. et. al., Gene 311, 153-163 (2003). The 2-DOG selection enriches for mutations that eliminate truncation of the GalK protein in E. coli using a galK Y145OFF mutation. Recombineering selections of the pooled chimeric libraries were transformed with plasmids that were designed to introduce a premature stop codon into the galK gene in E. coli. The galK gene encodes the galactose-kinase enzyme, which will metabolize 2-DOG into the toxic intermediate 2-deoxygalactose phosphate, which leads to cell death. Knockout constructs of this gene can thus be positively selected on 2-DOG minimal media plates supplemented with glycerol.

In brief, E. coli cells harboring the chimeric nuclease libraries were electroporated with plasmids containing a cassette for a GalK Y145OFF mutation, and allowed to recover for 3 hours. Selections were performed by transferring the cells at 3 hours post transformation into LB media with antibiotics to select for maintenance of the chimeric nuclease construct. After overnight recovery, 5 mL of saturated culture were concentrated to 100 μL and plated to M63 plates containing 0.2% 2-DOG and 0.2% glycerol. A control containing a nuclease that does not function with the cassette architecture was performed in parallel to monitor the rate of background mutations. The cells were allowed to grow overnight. Direct comparison of the number of viable cells at different times of growth after transformation allows one to distinguish between conditions where editing is expected at rates above background mutations.

Colonies that survived the above-described selection—and thus were presumed functionally active for editing capability—were picked and sequenced to confirm the presence of chimeric nuclease protein sequences by Sanger sequencing. The resultant clones were then purified from the edited colonies and reintroduced into naive MG1655 host cells and selected on plates containing chloramphenicol. These clones were subsequently screened by performing single plating on Mackonkey agar with 1% galactose.

The population of chimeric nucleases resulting from the 2-DOG selection were plated and individual colonies were isolated for follow up analyses including sequencing of the chimeric nuclease protein encoded on the plasmid. Colonies were picked from the 2-DOG selections and the GalK target region was sequenced to quantify editing. Sequence confirmation of the mutation of an editing region of an exemplary number of the mutated chimeric nucleases was performed, and each showed a mutation of the genome at the expected edit site.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCE LISTING

SEQ ID NO: 1
MTKTFDSEFFNLYSLQKTVRFELKPVGETASFVEDFKNEGLKRVVSEDERRAVDYQKV
KEIIDDYHRDFIEESLNYFPEQVSKDALEQAFHLYQKLKAAKVEEREKALKEWEALQKK
LREKVVKCFSDSNKARFSRIDKKELIKEDLINWLVAQNREDDIPTVETFNNFTTYFTGFH
ENRKNIYSKDDHATAISFRLIHENLPKFFDNVISFNKLKEGFPELKFDKVKEDLEVDYDL
KHAFEIEYFVNFVTQAGIDQYNYLLGGKTLEDGTKKQGMNEQINLFKQQQTRDKARQIP
KLIPLFKQILSERTESQSFIPKQFESDQELFDSLQKLHNNCQDKFTVLQQAILGLAEADLK
KVFIKTSDLNALSNTIFGNYSVFSDALNLYKESLKTKKAQEAFEKLPAHSIHDLIQYLEQF
NSSLDAEKQQSTDTVLNYFIKTDELYSRFIKSTSEAFTQVQPLFELEALSSKRRPPESEDE
GAKGQEGFEQIKRIKAYLDTLMEAVHFAKPLYLVKGRKMIEGLDKDQSFYEAFEMAYQ
ELESLIIPIYNKARSYLSRKPFKADKFKINFDNNTLLSGWDANKETANASILFKKDGLYYL
GIMPKGKTFLFDYFVSSEDSEKLKQRRQKTAEEALAQDGESYFEKIRYKLLPGASKMLP
KVFFSNKNIGFYNPSDDILRIRNTASHTKNGTPQKGHSKVEFNLNDCHKMIDFFKSSIQK
HPEWGSFGFTFSDTSDFEDMSAFYREVENQGYVISFDKIKETYIQSQVEQGNLYLFQIYN
KDFSPYSKGKPNLHTLYWKALFEEANLNNVVAKLNGEAEIFFRRHSIKASDKVVHPAN
QAIDNKNPHTEKTQSTFEYDLVKDKRYTQDKFFFHVPISLNFKAQGVSKFNDKVNGFLK
GNPDVNIIGIDRGERHLLYFTVVNQKGEILVQESLNTLMSDKGHVNDYQQKLDKKEQER
DAARKSWTTVENIKELKEGYLSHVVHKLAHLIIKYNAIVCLEDLNFGFKRGRFKVEKQV
YQKFEKALIDKLNYLVFKEKELGEVGHYLTAYQLTAPFESFKKLGKQSGILFYVPADYT
SKIDPTTGFVNFLDLRYQSVEKAKQLLSDFNAIRFNSVQNYFEFEIDYKKLTPKRKVGTQ
SKWVICTYGDVRYQNRRNQKGHWETEEVNVTEKLKALFASDSKTTTVIDYANDDNLID
VILEQDKASFFKELLWLLKLTMTLRHSKIKSEDDFILSPVKNEQGEFYDSRKAGEVWPK
DADANGAYHIALKGLWNLQQINQWEKGKTLNLAIKNQDWFSFIQEKPYQE
SEQ ID NO: 2
MNNGTNNFQNFIGISSLQKTLRNALIPTETTQQFIVKNGIIKEDELRGENRQILKDIMDDY
YRGFISETLSSIDDIDWTSLFEKMEIQLKNGDNKDTLIKEQTEYRKAIHKKFANDDRFKN
MFSAKLISDILPEFVIHNNNYSASEKEEKTQVIKLFSRFATSFKDYFKNRANCFSADDISSS
SCHRIVNDNAEIFFSNALVYRRIVKSLSNDDINKISGDMKDSLKEMSLEEIYSYEKYGEFI
TQEGISFYNDICGKVNSFMNLYCQKNKENKNLYKLQKLHKQILCIADTSYEVPYKFESD
EEVYQSVNGFLDNISSKHIVERLRKIGDNYNGYNLDKIYIVSKFYESVSQKTYRDWETIN
TALEIHYNNILPGNGKSKADKVKKAVKNDLQKSITEINELVSNYKLCSDDNIKAETYIHE
ISHILNNFEAQELKYNPEIHLVESELKASELKNVLDVIMNAFHWCSVFMTEELVDKDNN
FYAELEEIYDEIYPVISLYNLVRNYVTQKPYSTKKIKLNFGIPTLADGWSKSKEYSNNAII
LMRDNLYYLGIFNAKNKPDKKIIEGNTSENKGDYKKMIYNLLPGPNKMIPKVFLSSKTG
VETYKPSAYILEGYKQNKHIKSSKDFDITFCHDLIDYFKNCIAIHPEWKNFGFDFSDTSTY
EDISGFYREVELQGYKIDWTYISEKDIDLLQEKGQLYLFQIYNKDFSKKSTGNDNLHTM
YLKNLFSEENLKDIVLKLNGEAEIFFRKSSIKNPIIHKKGSILVNRTYEAEEKDQFGNIQIV
RKNIPENIYQELYKYFNDKSDKELSDEAAKLKNVVGHHEAATNIVKDYRYTYDKYFLH
MPITINFKANKTGFINDRILQYIAKEKDLHVIGIDRGERNLIYVSVIDTCGNIVEQKSFNIV
NGYDYQIKLKQQEGARQIARKEWKEIGKIKEIKEGYLSLVIHEISKMVIKYNAIIAMEDLS
YGFKKGRFKVERQVYQKFETMLINKLNYLVFKDISITENGGLLKGYQLTYIPDKLKNVG
HQCGCIFYVPAAYTSKIDPTTGFVNIFKFKDLTVDAKREFIKKFDSIRYDSEKNLFCFTFD
YNNFITQNTVMSKSSWSVYTYGVRIKRRFVNGRFSNESDTIDITKDMEKTLEMTDINWR
DGHDLRQDIIDYEIVQHIFEIFRLTVQMRNSLSELEDRDYDRLISPVLNENNIFYDSAKAG
DALPKDADANGAYCIALKGLYEIKQITENWKEDGKFSRDKLKISNKDWFDFIQNKRYL
SEQ ID NO: 3
MSQNIVDYCIGLDLGTGSVGWAVVDMNHRLMKRNGKHLWGSRLFSNAETAANRRAS
RSIRRRYNKRRERIRLLRAILQDMVLENDPTFFIRLEHTSFLDEEDKANYLGADYKDNYN
LFIDEDFNDYTYYHKYPTIYHLRKALCESTEKADPRLIYLALHHIVKYRGNFLYEGQKFN
MDASNIEDRLSDVFTQFADFNNIPYEDDEKKNLEILEILKKPLSKKAKVDEVMALIAPEK
DFKSAYKELVTGIAGNKMNVTKMILCEPIKQGDSEIKLKFSDSNYDDQFSEVENDLGEY
VEFIDSLHNIYSWVELQTIMGATHTYNASISEAMVSRYNKHHEDLQLLKKCIKDNVPKK
YFDMFRNDSEKLKGYYNYINHPSKAPVDEFYKYVKKCIEKVDTPEAKQILHDIELENFL
LKQNSRTNGSVPYQMQLDEMIKIIDNQAKYYPVLKEKREQLLSILTFKIPYYFGPLNETS
EHAWIKRLEGKENQRILPWNYQDIVDVDATAEGFIKRMRSYCTYFPDEEVLPKNSLIVS
KYEVYNELNKIRVDDKLLEVDIKNDIYNELFMNNKTVTEKKLKNWLVNNQCCNKNAEI
KGFQKENQFSTSLTPWIDFTNIFGEINQSNFDLIEDITYDLTVFEDKKIMKRRLKKKYALP
DDKIKQILKLKYKDWSRLSKKLLDGIVADNKFGSSVTVLDVLEMSRLNLMEIINDRDLG
YAQMIEAAASCPEDGKFTYKEVQRLAGSPALKRGIWQSLQIVEEITKVMKCRPKYIYIEF
ERSEETKERTESKIKKLENVYKDLDEQTKVEYKTVLEELKGFDNTKKISSDSLFLYFTQL
GKCMYSGKKLDIDSLDKYQIDHIVPQSLVKDDSFDNRVLVVPSENQRKLDDLVVPSDIR
VKMNSFWKLLFDHELISPKKFYSLIKTEYTERDEERFINRQLVETRQITKNVTQIIEDHYS
TTKVAAIRANLSHEFRVKNHIYKNRDINDYHHAHDAYIVALIGGFMRDRYPNMHDSKA
VYSEYMKMFRKNKNDKKRWKDGFVINSMNYPYEVDGELIWNPDIINEIRKCFYYKDCY
CTTKLDQKSGQMFNLTVLPNDAHSPKGTTEAVIPVNKNRKDVNKYGGFSGLQYVIVAIE
GKKKRGKKTKLVKKISGVPLHLKAASLDEKIKYIEEKENLTDVKIIKDSIPVNQMIEMDG
GEYLLTSPIEFVNGRQLVLNEKQCALIADIYNAIYKQDCDNLDDVLMIQLYIELINKMKA
LYPAYQSIAEKFESMTEDYVAVSKEEKADIIKQMLIIMHRGPRNGKIQYADFNVGDRIGR
KNKMSLDLERVTFVSQSPTGIYTKKYKL
SEQ ID NO: 4
MSQNNNKIYNIGLDIGDASVGWAVVDEHYNLLKRHGKHMWGSRLFTQANTAVERRSS
RSTRRRYNKRRERIRLLREIMEDMVLDVDPTFFIRLANVSFLDQEDKKDYLKENYHSNY
NLFIDKDFNDKTYYDKYPTIYHLRKHLCESKEKEDPRLIYLALHHIVKYRGNFLYEGQKF
SMDVSNIEDKMIDVLRQFNEINLFEYVEDRKKIDEVLNVLKEPLSKKHKAEKAFALFDT
TKDNKAAYKELCAALAGNKFNVTKMLKEAELHDEDEKDISFKFSDATFDDAFVEKQPL
LGDCVEFIDLLHDIYSWVELQNILGSAHTSEPSISAAMIQRYEDHKNDLKLLKDVIRKYL
PKKYFEVFRDEKSKKNNYCNYINHPSKTPVDEFYKYIKKLIEKIDDPDVKTILNKIELESF
MLKQNSRTNGAVPYQMQLDELNKILENQSVYYSDLKDNEDKIRSILTFRIPYYFGPLNIT
KDRQFDWIIKKEGKENERILPWNANEIVDVDKTADEFIKRMRNFCTYFPDEPVMAKNSL
TVSKYEVLNEINKLRINDHLIKRDMKDKMLHTLFMDHKSISANAMKKWLVKNQYFSNT
DDIKIEGFQKENACSTSLTPWIDFTKIFGKINESNYDFIEKIIYDVTVFEDKKILRRRLKKE
YDLDEEKIKKILKLKYSGWSRLSKKLLSGIKTKYKDSTRTPETVLEVMERTNMNLMQVI
NDEKLGFKKTIDDANSTSVSGKFSYAEVQELAGSPAIKRGIWQALLIVDEIKKIMKHEPA
HVYIEFARNEDEKERKDSFVNQMLKLYKDYDFEDETEKEANKHLKGEDAKSKIRSERL
KLYYTQMGKCMYTGKSLDIDRLDTYQVDHIVPQSLLKDDSIDNKVLVLSSENQRKLDD
LVIPSSIRNKMYGFWEKLFNNKIISPKKFYSLIKTEFNEKDQERFINRQIVETRQITKHVAQ
IIDNHYENTKVVTVRADLSHQFRERYHIYKNRDINDFFIHAHDAYIATILGTYIGHRFESL
DAKYIYGEYKRIFRNQKNKGKEMKKNNDGFILNSMRNIYADKDTGEIVWDPNYIDRIK
KCFYYKDCFVTKKLEENNGTFFNVTVLPNDTNSDKDNTLATVPVNKYRSNVNKYGGFS
GVNSFIVAIKGKKKKGKKVIEVNKLTGIFLMYKNADEEIKINYLKQAEDLEEVQIGKEIL
KNQLIEKDGGLYYIVAPTEIINAKQLILNESQTKLVCEIYKAMKYKNYDNLDSEKIIDLYR
LLINKMELYYPEYRKQLVKKFEDRYEQLKVISIEEKCNIIKQILATLHCNSSIGKIMYSDF
KISTTIGRLNGRTISLDDISFIAESPTGMYSKKYKL
SEQ ID NO: 5
MAKKDYTIGLDIGTNSVGWAIIDDNLKLLKRNMTIKGNTDKKSVKRDLWGSLLYSGNS
DKTTSAADARSKRGLRRRLRRRKYRLDRLKQIFSEIINDKAPNFFDKLNESFLNPKDKKY
GKYQIFDTEKEEKDYYRRYPTIYHLRKDLIESSKKQDIRLVYLALAHILKSRGNFLFEGNI
DDLKNDFAGIYEEVVELCMTINAEDVDLEFEEVDKQSLNSIIKNEDISEIEQGLENFADEH
VIFKEQNKKKNDLFSNCCKIICGHTVKANKFASELDSELFISFKSDDYVDVIDVIQSGNEN
IANLLLACRKAYDYIMFNRLVDLNIDSPAKLSSNMVSLYNQHEKDLKAYKKLIKEFNKF
KRSNGCKDLEMIILTADDIDSFRKKVDKKEGKLNGINKKITHEQALKKQLKDMKKILED
KNTEAEDKQINDILKMITSIEERVNKSCFLKNLRSTDNASIPNQIQRQEMEAILDKQAKFY
PFLNEHKDELLQLLSFRIPYYVGPLVNKKYSRFAWLVRKEGQVQKITPTNFDGVVDKHK
TAEKFMERLIGKDVYLPNERVLPKASLLYQEYCIFNELTKVAYIDSTGKKNNFSSEEKLN
IFEKLFKTKREVTKTDLCKCLNNVCKLKEKVKETEIIGIKAKFNAKYSTYHDLKKINGME
QLIADEEGKPLCEDIISILTIFEDKDIRLVRLKELLCQNKDLINKFSLSAEKLAKVLSTKHY
KGFGNVSAKLINGIRDKNCKTILDYLIEDDKEAYYGRNNPNRNLMQLVNDSRLAFKGQI
DREQNTHLEDLSLDEFLDDLYVSPSIRRGIRLTIRLVDELVEIMGYLPKNIVIEMPREDGE
KGKIADTRYSKLEKMLKKDAALEDLYRVLKTYEKNKKALANDALYLYFLQNGRDMYT
GKEINLSELHSYDIDHIIPKSFKYDDSLDNKVLTAKKMNMDKRTGALDHNIIENQCGFW
RVLLQQDKISLEKYTNLMKTEFTEADKAGFIMRQLVETRQITKFVARYLDNKFNGLISDP
NDKVNILLPRASLCHQFRETFGFYKVRELNDMHHAHDAYLNAVIANTLNKNAYLSDLL
KYGAYSKYKKNGFNNSNGIMDYFGNTQFNCLFVVERTLDKCRVNIVKHPETASGEFYN
ETIQKNKVNGGSSTRSLKSSVKVLQNTEQYGGFTNVNNAYFILFDYKAKSKLKRKLIGV
PIVDRQKFEQDPVTYLEAKGFDEPKLVQKLLKYTLLEYEDGKRRYLTGVTGKRCELVR
ANQLLLPRNMMALLHHLQEWQKHDFGIKEMTKVIKNTNNIEAKFDKLFEHMMKFIDK
YSEPPKIVSSKISEEYHKLRESLCQDDNKIKIYAEIGKALLSLLHLVDSKSACVFKFSGLEI
NRIRYQSINEKKEPVIIFQSLSGLRESRYKYNQ
SEQ ID NO: 6
MRDYYIGLDLGTGSLGWAVTDREYEIMRAHGKALWGVRLFDSANTAEERRGFRTARR
RLDRRNWRIELLQELFGEDIGKVDSGFFLRMKESKYMPEDKRDVNGNCPKLPYALFVE
DGYTDKDYHRQFPTIYHLRKWLMETEETPDIRLVYLALHHMMKHRGHFLFSGNIEKIKE
FQETFRQYIGKIREEELDFHLCIEGEELRETENILKDKNLTRSAKKTRLIKLLGAHTACEK
AALNLVAGGTVKLSDIFGNSELDACEKPKLSFADAGYDDYAGMIEDELGEQHVIIETAK
AVYDWSVLADILGDYRCISEAKAAVYEKHQKDLRHLKELVKENLGRDVYKEVFVKTN
EKLPNYSAYIGMTKKNGVKSEMEGKRCDRKAFYDYLKKTVVNAIPDESKTEYLRKEME
TETFLPRQVTKDNGVIPHQVHLQELDAILENLSGRIPALKENGSKIRDIFTFRIPYYVGPLN
GIVKGGERTNWVRRKKAGRICPWNFDEMVDTGASAEEFIRRMTSKCTYLIHEDVLPKN
SMLYSKFMVLNELNNVRLNGEPISVELKQKTYEDLFQRHRKVTRRRLTDYIRREGIAGR
DADITGIDGDFKGSLTAYHDFKEKLTGCELSQADKENIILNITLFGEDKALLKKRLGALY
PALTEPQKKAICALSYKGWGRLSQRLLEGITAPAPETGEIWTVIRAMWETNDNLMQVLS
EKYCFAAAIDEENAGEELKEITYKTVEQMNVSPAVRRQIWQSLQVIKEICKVMGGPPKR
VFVEMAREKMESKRTESRKKRLIDLYKKCREEERDWIEELGNTEETRLRSDKLYLYYTQ
KGRCMYSGEVIELEELWDNRKYDIDHIYPQSKVMDDSLDNRVLVKKEYNADKTDEYPI
RADIRGKMRAFWRILREEGFISKEKYNRLTRGTGFEPSELAGFIARQLVETRQGTKAVAS
VLKQVFPETDIVYAKARVASQFRQEFDLIKVREMNDLHHAKDAYVNIVVGNVYYTKFT
SNAAWYVKEHPGRSYNLKKMFTSERDVARNGETAWRAGNSGTIATVKRVMGKNNILV
TRRSYEVKGGLFDQQLMKKGKGQVPIKGRDERLADIDKYGGYNKAAGTYFMLAESED
KKGAKIRSVEYVPLYLCNCIEKDEEAAKKYLQKERGLKNPRVLIAKIKIDTLFKVDGFY
MWLSGRTGNQLIFKGANQLILSEPDMRILKKVLKYVNRKKENKNAVLGEHDQLPETDLI
RLYDVFLDKIENTVYHVRLSAQQGTLTKNKDTFCELSNEDKCIVLSEILHMFQCQSGSA
NLKLIKGPGSAGILVLNNIISKCNQVSIIHQSPTGIYEQEIDLKKI
SEQ ID NO: 7
MEQEYYLGLDMGTGSVGWAVTDSEYHVLRKHGKALWGVRLFESASTAEERRMFRTSR
RRLDRRNWRIEILQEIFAEEISKKDPGFFLRMKESKYYPEDKRDINGNCPELPYALFVDD
DFTDKDYHKKFPTIYHLRKMLMNTEETPDIRLVYLAIHHMMKHRGHFLLSGDINEIKEF
GTTFSKLLENIKNEELDWNLELGKEEYAVVESILKDNMLNRSTKKTRLIKALKAKSICEK
AVLNLLAGGTVKLSDIFGLEELNETERPKISFADNGYDDYIGEVENELGEQFYIIETAKAV
YDWAVLVEILGKYTSISEAKVATYEKHKSDLQFLKKIVRKYLTKEEYKDIFVSTSDKLK
NYSAYIGMTKINGKKVDLQSKRCSKEEFYDFIKKNVLKKLEGQPEYEYLKEELERETFLP
KQVNRDNGVIPYQIHLYELKKILGNLRDKIDLIKENEDKLVQLFEFRIPYYVGPLNKIDD
GKEGKFTWAVRKSNEKTYPWNFENVVDIEASAEKFIRRMTNKCTYLMGEDVLPKDSLL
YSKYMVLNELNNVKLDGEKLSVELKQRLYTDVFCKYRKVTVKKIKNYLKCEGIISGNV
EITGIDGDFKASLTAYHDFKEILTGTELAKKDKENIITNIVLFGDDKKLLKKRLNRLYPQI
TPNQLKKICALSYTGWGRFSKKFLEEITAPDPETGEVWNIITALWESNNNLMQLLSNEYR
FMEEVETYNMGKQTKTLSYETVENMYVSPSVKRQIWQTLKIVKELEKVMKESPKRVFI
EMAREKQESKRTESRKKQLIDLYKACKNEEKDWVKELGDQEEQKLRSDKLYLYYTQK
GRCMYSGEVIELKDLWDNTKYDIDHIYPQSKTMDDSLNNRVLVKKKYNATKSDKYPL
NENIRHERKGFWKSLLDGGFISKEKYERLIRNTELSPEELAGFIERQIVETRQSTKAVAEIL
KQVFPESEIVYVKAGTVSRFRKDFELLKVREVNDLHHAKDAYLNIVVGNSYYVKFTKN
ASWFIKENPGRTYNLKKMFTSGWNIERNGEVAWEVGKKGTIVTVKQIMNKNNILVTRQ
VHEAKGGLFDQQIMKKGKGQIAIKETDERLASIEKYGGYNKAAGAYFMLVESKDKKGK
TIRTIEFIPLYLKNKIESDESIALNFLEKGRGLKEPKILLKKIKIDTLFDVDGFKMWLSGRT
GDRLLFKCANQLILDEKIIVTMKKIVKFIQRRQENRELKLSDKDGIDNEVLMEIYNTFVD
KLENTVYRIRLSEQAKTLIDKQKEFERLSLEDKSSTLFEILHIFQCQSSAANLKMIGGPGK
AGILVMNNNISKCNKISIINQSPTGIFENEIDLLKI
SEQ ID NO: 8
MKQEYFLGLDMGTGSLGWAVTDSTYQVMRKHGKALWGTRLFESASTAEERRMFRTA
RRRLDRRNWRIQVLQEIFSEEISKVDPGFFLRMKESKYYPEDKRDAEGNCPELPYALFVD
DNYTDKNYHKDYPTIYHLRKMLMETTEIPDIRLVYLVLHHMMKHRGHFLLSGDISQIKE
FKSTFEQLIQNIQDEELEWHISLDDAAIQFVEHVLKDRNLTRSTKKSRLIKQLNAKSACE
KAILNLLSGGTVKLSDIFNNKELDESERPKVSFADSGYDDYIGIVEAELAEQYYIIASAKA
VYDWSVLVEILGNSVSISEAKIKVYQKHQADLKTLKKIVRQYMTKEDYKRVFVDTEEK
LNNYSAYIGMTKKNGKKVDLKSKQCTQADFYDFLKKNVIKVIDHKEITQEIESEIEKENF
LPKQVTKDNGVIPYQVHDYELKKILDNLGTRMPFIKENAEKIQQLFEFRIPYYVGPLNRV
DDGKDGKFTWSVRKSDARIYPWNFTEVIDVEASAEKFIRRMTNKCTYLVGEDVLPKDS
LVYSKFMVLNELNNLRLNGEKISVELKQRIYEELFCKYRKVTRKKLERYLVIEGIAKKG
VEITGIDGDFKASLTAYHDFKERLTDVQLSQRAKEAIVLNVVLFGDDKKLLKQRLSKMY
PNLTTGQLKGICSLSYQGWGRLSKTFLEEITVPAPGTGEVWNIMTALWQTNDNLMQLLS
RNYGFTNEVEEFNTLKKETDLSYKTVDELYVSPAVKRQIWQTLKVVKEIQKVMGNAPK
RVFVEMAREKQEGKRSDSRKKQLVELYRACKNEERDWITELNAQSDQQLRSDKLFLYY
IQKGRCMYSGETIQLDELWDNTKYDIDHIYPQSKTMDDSLNNRVLVKKNYNAIKSDTYP
LSLDIQKKMMSFWKMLQQQGFITKEKYVRLVRSDELSADELAGFIERQIVETRQSTKAV
ATILKEALPDTEIVYVKAGNVSNFRQTYELLKVREMNDLHHAKDAYLNIVVGNAYFVK
FTKNAAWFIRNNPGRSYNLKRMFEFDIERSGEIAWKAGNKGSIVTVKKVMQKNNILVTR
KAYEVKGGLFDQQIMKKGKGQVPIKGNDERLADIEKYGGYNKAAGTYFMLVKSLDKK
GKEIRTIEFVPLYLKNQIEINHESAIQYLAQERGLNSPEILLSKIKIDTLFKVDGFKMWLSG
RTGNQLIFKGANQLILSHQEAAILKGVVKYVNRKNENKDAKLSERDGMTEEKLLQLYD
TFLDKLSNTVYSIRLSAQIKTLTEKRAKFIGLSNEDQCIVLNEILHMFQCQSGSANLKLIG
GPGSAGILVMNNNITACKQISVINQSPTGIYEKEIDLIKL
SEQ ID NO: 9
MQQYYLGVDMGSASVGWAVTDEKYQLVRKKGKDLWGVRTFDIAQTAEVRRVSRTNR
RRQNRRKQRIQILQELLGEEVLKIDAGFFHRMKESRYVAEDKRTLDGKQVELPYALFVD
QGFTDKDFYKQFPTINHLIVYLMTTSDTPDIRLVYLALHYYMKNRGNFLHSGDINDVKD
IQSILEQLENVLKEYVDDWELSLKDKVDAIKEIYNKDLGRGERKKAFINTLGVKTKSAK
AFCSLISGGSTNLAELFDDSGLKESEYAKIEFANANFEDSVEGIQALLEDRFAVIEAAKRL
YDWKILTDILGDNASLAEARVKSYETHHEQLVELKSFIKKYLDRKIYQDIFINPNIANNYP
AYVGHTKINGKKQELEVKRAKRNDFYAYIKKQVIDPIKKKVSDKAVLARLAEIESLIEV
NKYLPLQVNSDNGVIPYQIKLNELRRIFNNLENRLPVLKENRDKIIKTFSYRIPYYVGPLN
GVNRNGKSTNWMVRKEGEEGKIYPWNFEEKVDLEASAEKFIRRMTNKCTYLVNEDVL
PKYSLLYSKYLVLSELNNLRLDGRPLEVSVKQEIYENVFKRNRKVTLKKIKNYLLKEGVI
SEKDELSGLADDVKSSLTAYHDFKEKLGHLTLTEDQMEKIILNVTLFGDDKKLLKKRLA
ALYPNIDEKSLSRMATFNYRDWGRLSKKFLSEITSVDQETGELRTIIQCMYETQNNLMQ
LLSEPYHFVEAIEKENPKVDLESISYRIVNDLYVSPAVKRQIWQTLLVIKDIKQVMKHDP
KRIFIEMAREKQESKTTKSRKQVLSEVYKNAEKYKNLFEKLNSLTEEQLRSKKVYLYFT
QLGKCMYTNDAIDFENLVSANSNYDIDHIYPQSKTIDDSFNNLVLVKKGINNDKSDRYPI
DKNIRDDEKVKTLWNTLLSKGLITKEKFERLIRSTPFSDEELAGFIARQLVETRQSTKAV
AEILSNWFPESEIVYSKAKHITNFRQDFEILKVRELNDCHHAHDAYLNIVVGNAYHTKFT
NSPYRFIQNKANQEYNLRKLLQKAKKIESNGVIAWIGQSENNPGTIATVKKVISRNTVLIS
RMVKEVDGQLFDQQLMKKGKGQVPIKSSDDRLIDISKYGGYNKAKGAYFVFIKSVRRG
KTIKSFEYIPVHLAKKFDCNLELLKEYLESEKDLNNVEILMPKVMINSLFNYNGSLIRIPG
RYDKKSLLINVDVPLLLESQHIKQLKVIEKYMYKKRVSKNSNILLTKFASDQLKDLDALF
DVLSYKLNENIYNVINDKYDKLVICRDKFISLDTEVKCEMIFELLHLFQCNSQLANITKIG
ATSKFGSISMSKNLKENDKMSIIHQSPSGIFEHEIELTAL
SEQ ID NO: 10
MGYNIGLDIGTGSVGWAALTDEGKLARAKGKNLIGVRLFDSAQSAAQRRSYRTTRRRL
SRRKWRLRLLENIFSDEMGMIDENFFARLKYSYVHPKDEVNNAHYYGGYLFPTQQETH
DFHEKFQTIYHLRLKLMIEDCKFDLREIYLAMHHIVKYRGHFLNSQSKMTIGDSYNPRDF
QQAIQNYAEAKGLIWSLNDAQEMTDVLVGQAGFGLSKKAKAERLLSAFSFDTKEDKKA
IQAILAGIVGNTTDFTKIFNRERSGDELKKWKLKLDSEAFDEQSQAIVDELDDDEMELFN
AIRQAFDGFTLMDLLGDQTSISAAMVKRYQQHHDDLKMVKEIAKKQGLSHQDFSKIYT
AFLKDDTDKGMKALLDKADLADDVLVEIQQRIESHDFLPKQRTKANSVIPYQLHLAELE
KIIENQGKYYPFLLDTFTNKAGETINKLVELVKFRVPYYVGPMVTAADVEKAGGDATN
HWVKRNEGYEKSPVTPWNFDQVFNRDQAAQDFIDRLTGTDTYLIGEPTLLKNSLKYQL
FTVLNELNNVKINGHKIDEKTKHVLIQDLFKSKKTVSEKAIKDYYLSQGMGEIQIVGLAD
KTKFNSNLSSYIDLSKTFDAEFMENPANQELLENIIQIQTVFEDVKIAERELQKLALPDEQ
VQQLAKTHYTGWGNLSDKLLSTPIIQEGSQKVSILNKLQTTSKNFMSIITDNKFGVQQWI
QEQNTAETADSIQDRIDELTTAPANKRGIKQAFNVLFDIQKAMGEEPNRVYLEFAKETQ
NSVRTNSRYNRLKDLYKSKTLSDDVKALKEELESQKSSLQSERIGDRLYLYFLQQGKDM
YTGQPINIDKLSTDYDIDHIIPQAYTKDDSIDNRVLVSRPENARKSDSATYTTEVQQSAGG
LWKSLKNAGFISQKKYDRLTKGGDYSKGQKTGFIARQLVETRQIIKNVASLIESEFSQTK
AVAIRSEITADMRRLVAIKKHREINSFHHAFDALLITAAGQYMQARYPDRDGANVYNEF
DYYTNTYLKELRQSSSSSQVRRLKPFGFVVGTMAKGNENWSEDDTQYLRHVMNFKNIL
TTRRNDKDNGALNKETIYAVDPKAKLIGTNKKRQDVSLYGGYIYPYSAYMTLVRANGK
NLLVKVTISAAEKIKSGQIELSEYVQQRPEVKKFEKILINKLAIGQLVNNDGNLIYLTSYE
FYHNAKQLWLPTEEADLISQLNKDSSDEDLIKGFDILTSPAILKRFPFYELDLKKLVNIRD
KFIAVENKFDILMVILKALQLDAAQQKPVKMIDKKSADWKDYRQRGGIKLSDTSEIIYQ
STTGIFEKRVKISNLL
SEQ ID NO: 11
MAYSVGLDIGVGSVGFAGIDNQYNLVRTKGKNVIGVRLFDEADSAAERRGHRTNRRRL
QRRRWRLRLLDDIFAKPLQAVDPNFLARLKYSYVNKKDQGQQDHYYGGYVFGSTAAD
QAYHQAYPTIYHLRKRLMEDDQKHDLREVYLAIHHIVKYRGNFLNPQSSLDIDQQFDVT
DFAQALARFADHQALSWALEAPIRFLEAELATGLSNSARVDAAIEAFSFDTKVDRAAIK
EMLKGLSGNQIDFTKLFVNVDSADWDQEERKQWKMKLSEEDFDEQALPILERLSQDET
EFFLAIKRAYDGIALMRFLGDEQSLSSAMIKAYEDHRRDLTFLKTQVRTPQNRQALSEG
YTNYLSVDDKKHKRGAKELAQLIEASDASEQDKATMLDRIANDQFAPKQRTKANGLIP
YQLHLAELKKILAKQGQYYPFLLDTFAKQGQSVNKIEELVQFRVPYYVGPMVPKSETA
GNAENHWVEKNDGQTKVSVTPWNFDQVFNRDRAAKSFIDRLTGTDTYLIGEPTLPRHS
LTYETFTVLNELNNIRIDGKRLPVETKQAIVEDLFKKYRLVTKKRLQDYFASFGKREVEL
TGLADESRFTSSLTSYHDLQGLLGTDFITNPQNHSLLEKIVEIQTVFEDSDIAERELGKLG
LEQKLIPRLAKKHYTGWGNLSRKLLDTSFIHDPERPEEPVSIMDLLYTTNKNFMEILHDS
EYGVEEWLKSQNMIDDQKDIQMRIDELTTSPANKRGIKQAFNVLDDITQAMGEEPAYV
YLEFAREKQASRRTVSRKKRLETLYKNAALKTEFKAIKEALAEESDDRMQDDRLYLYY
AQLGRDMYTGQSISIDQLSSHYDIDHIVPRAFIKDDSLENKVLVNRTDNARKTDSATFTA
DVKAKAFPLWQQLKKLGLISAKKFRLLTRTGDFTEMERERFIARQLVETRQIIKNVAALI
EGHFSQTQAVAIRAEVTGELRQLTQIKKDRDINDYHHAQDALLVATAGTYLHRHFPKR
DARFIYNEFDYYTQHWLKNQGENRRRHPYSFVVGTMSKGNEDWTPDNLNYLRKVMQ
YKTMLMTRKPVGPEGALYKETLIAADPKKRLVGASKERQDPTIYGGYTKESSAYMSLV
RAGGKNQLVKIPVRIANEIHSGQRKLDDYVQAKVKKFERILLPKISLGQLVEDEGQRFYL
ATNEMKHNAKQLWLDQKVVTTYKRLTAESPVEDFLTVFDALTSSATIHHFKFYQRDLE
LLRDNRAGFQDLAKATQLKVLKDVLYELHDNAGWRDPIKQYFKEIGLKVRMWTKLQK
EGGIKLTDQAELIYQSPSGLFEKRRRVQDLL
SEQ ID NO: 12
MGDRKYNLGLDIGTSSIGFAAVDENNQPIRVKGKTAIGVRLFEEGKTAADRRGFRTTRR
RLSRRRWRINLLNEIFDAHLAEVDPTFLARLKESNRSNLDPKKSFQGSLLFPERKDYQFY
EEYPTIYHLRKALMEKDRKFDFREIYLAVHHIIKYRGNFLNGTPMRSFKVENIELDTLFD
QLNQLYAEIVPDNELAFDLAQVADVKDVLSSTTIYKMDKKKQLVKMMLLPASNKALQ
SENKKIVTQFVNAILNYKFKLDVLLQVETDADWSLKLNDEGADDKLEEFTGDLDENRL
EIIDLLQRLHNWFSLNEITKDGNSLSAAMVEKYENHHHHLGLLKKVIENHPDAKKAKAL
KETYTAYVGKTDDKTQNQDDFYKAVEKNLDDSPDAKEIKRLIQLDQFMPKQRTGQNG
AIPHQLHQQELDQIIEKQSKYYPFLAEPNPNVKRRKDAPYKLDELIAFKIPYYVGPLVTPE
EQAQNGENVFAWMKRKAAGPITPWNFDEKVDRMESANRFIRRMTTKDTYLFGEDVLP
AESMIYQKFVVLNELNNLKINGRHLSLKDKQDVYNDLFKQQKTVSIKALQNYYVTKKK
AATAPTVGGLADPKKFLSSLSTYIDFKNMFGERVNDPQFQEDLEQIVEWSTIFEDRGIFK
AKLQALGWLSEKQIQQLVAKRYKGWGRLSKKLLTGLKNAEGYSILDEMWRSTGNFMQ
IQSRPEFAALIQQANEKQFEGNDPDNVWENIENILGDAYTSPQNKKAIRQVVKVVQDIEK
AVGNPPEKIAIEFTREAAANPQRTQSRLRTLEKLYESAEEVVDAGLTAELAEFKENKHVL
SDKYYLYFTQLGRDVYTGDTISLDKLNDYDVDHILPQSFIKDDSLDNRVLTIRAVNNGK
SDNVPAKMFGKKMGSFWRYLLDNGMISKRKYNNLITDPDNISKYAQKGFINRQLVETS
QVIKLTANILNGIYDKDTEIIEVPAKMNSQMRKMFDLVKVREVNDYHHAFDAYLTIFIG
NYLYKCYPKLQPYFVYDNFKKFGNKEDIGHKRFNFLGKIEREKKVVAPETGEILWSNVA
PNETIKQIKKVYDYKFMIVSREITTRRAELFNQTVYPKNYHGKLIPIKEDRPTDLYGGYS
GNTDAYLAIVALEDKKKGKYFKVVGIPTRVAAKLEKLKQQDSQQYLQALHKVIAPQFT
KSTKKGIKKTEFEIVLDKVHYRQLVQDGPVKMMLGSSTYKYNAKQLVLSEKALQVIAD
DRKFDETQKDDNLIAVYDEILSIVNQSFDLYDINGFRKKLNDNRDQFIDLPAETKYEGRK
VVAHGKREMILEILKGLHANAAFGNLKPIGFSTAFGQLQVPNGIILSKNAILIHQSPSGLF
ERKIKLSDL

| | |
|---|---|
| SEQ ID NO: 13 | CTCTAGCAGGCCTGGCAAATTTCTACTGTTGTAGAT |
| SEQ ID NO: 14 | GTTAAGTTATATAGAATAATTTCTACTGTTGTAGA |
| SEQ ID NO: 15 | ACTACATTTTTTAAGACCTAATTTTGAGT |
| SEQ ID NO: 16 | CTCAAAACTCATTCGAATCTCTACTCTTTGTAGAT |
| SEQ ID NO: 17 | GTCTAAAACTCATTCAGAATTTCTACTAGTGTAGAT |
| SEQ ID NO: 18 | GTCTAGGTACTCTCTTTAATTTCTACTATTGT |
| SEQ ID NO: 19 | GTTTAAAACCACTTTAAAATTTCTACTATTGTA |
| SEQ ID NO: 20 | ATAATAATTTCTACTTTTGTAGAT |
| SEQ ID NO: 21 | ATCTACAATAGTAGAAATTTTTAAAAACGATTTGAC |
| SEQ ID NO: 22 | ATCTACAATAGTAGAAATTTTTAAAAACGATTTGAC |
| SEQ ID NO: 23 | GTCTAACGACCTTTTAAATTTCTACTGTTTGTAGA |
| SEQ ID NO: 24 | GTTTGAGAGATATGTAAATTCAAAGGATAATCAAAC |
| SEQ ID NO: 25 | GGTTTTAGAGTTGTGTTATTTTGAACAGATACAAAAC |
| SEQ ID NO: 26 | GCTTGTGTACCATACATTTTTACATCATTCTCAAAC |
| SEQ ID NO: 27 | GTTTGAGAATGATGTAAAAATGTATGGTACTCAAGC |
| SEQ ID NO: 28 | GCTTTAGATGTATGTCAGATTAATGGGGTTTATTCC |
| SEQ ID NO: 29 | GTTTCAGAAGGATGTTAAATCAATAAGGTTAAGATCTT |

SEQ ID NO: 30
MTQFEGFTNLYQVSKTLRFELIPQGKTLKHIQEQGFIEEDKARNDHYKELKPIIDRIYKTY
ADQCLQLVQLDWENLSAAIDSYRKEKTEETRNALIEEQATYRNAIHDYFIGRTDNLTDAI
NKRHAEIYKGLFKAELFNGKVLKQLGTVTTTEHENALLRSFDKFTTYFSGFYENRKNVF
SAEDISTAIPHRIVQDNFPKFKENCHIFTRLITAVPSLREHFENVKKAIGIFVSTSIEEVFSFP
FYNQLLTQTQIDLYNQLLGGISREAGTEKIKGLNEVLNLAIQKNDETAHHASLPHRFIPLF
KQILSDRNTLSFILEEFKSDEEVIQSFCKYKTLLRNENVLETAEALFNELNSIDLTHIFISHK
KLETTSSALCDHWDTLRNALYERRISELTGKITKSAKEKVQRSLKHEDINLQEIISAAGKE
LSEAFKQKTSEILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVFRDGSNILPKLLE
NDDSHAIDTMVALIRSVLQMRNSNAATGEDYINSPVRDLNGVCFDSRFQNPEWPMDAD
ANGAYHIALKGQLLLNHLKESKDLKLQNGISNQDWLAYIQELRN
SEQ ID NO: 31
MDKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD″

| | |
|---|---|
| SEQ ID NO: 32 | GTTTTAGAAGAGTATCAAATCAATGAGTAGTTCAAC |
| SEQ ID NO: 33 | GTTTGACTACCATATGAAATTACACTACTCTCAAAC |
| SEQ ID NO: 34 | PKKKRKV |
| SEQ ID NO: 35 | KRPAATKKAGQAKKKK |
| SEQ ID NO: 36 | PAAKRVKLD |
| SEQ ID NO: 37 | RQRRNELKRSP |
| SEQ ID NO: 38 | NQSSNFGPMKGGNFGGRSSGPYGGGGQYFAKPRNQGGY |
| SEQ ID NO: 39 | RMRIZFKNKGKDTAELRRRRVEVSVELRKAKKDEQILKRRNV |
| SEQ ID NO: 40 | VSRKRPRP |
| SEQ ID NO: 41 | PPKKARED |
| SEQ ID NO: 42 | PQPKKKPL |
| SEQ ID NO: 43 | SALIAP |
| SEQ ID NO: 44 | DRLRR |
| SEQ ID NO: 45 | PKQKKRK |
| SEQ ID NO: 46 | RKLKKKIKKL |
| SEQ ID NO: 47 | REKKKFLKRR |
| SEQ ID NO: 48 | KRKGDEVDGVDEVAKKKSKK |
| SEQ ID NO: 49 | RKCLQAGMNLEARKTKK |

SEQ ID NO: 50
MSSLTKFTNKYSKQLTIKNELIPVGKTLENIKENGLIDGDEQLNENYQKAKIIVDDFLRDF
INKALNNTQIGNWRELADALNKEDEDNIEKLQDKIRGIIVSKFETFDLFSSYSIKKDEKIID
DDNDVEEEELDLGKKTSSFKYIFKKNLFKLVLPSYLKTTNQDKLKIISSFDNFSTYFRGFF
ENRKNIFTKKPISTSIAYRIVHDNFPKFLDNIRCFNVWQTECPQLIVKADNYLKSKNVIAK
DKSLANYFTVGAYDYFLSQNGIDFYNNIIGGLPAFAGHEKIQGLNEFINQECQKDSELKS
KLKNRHAFKMAVLFKQILSDREKSFVIDEFESDAQVIDAVKNFYAEQCKDNNVIFNLLN
LIKNIAFLSDDELDGIFIEGKYLSSVSQKLYSDWSKLRNDIEDSANSKQGNKELAKKIKTN
KGDVEKAISKYEFSLSELNSIVHDNTKFSDLLSCTLHKVASEKLVKVNEGDWPKHLKNN
EEKQKIKEPLDALLEIYNTLLIFNCKSFNKNGNFYVDYDRCINELSSVVYLYNKTRNYCT
KKPYNTDKFKLNFNSPQLGEGFSKSKENDCLTLLFKKDDNYYVGIIRKGAKINFDDTQAI
ADNTDNCIFKMNYFLLKDAKKFIPKCSIQLKEVKAHFKKSEDDYILSDKEKFASPLVIKK
STFLLATAHVKGKKGNIKKFQKEYSKENPTEYRNSLNEWIAFCKEFLKTYKAATIFDITT
LKKAEEYADIVEFYKDVDNLCYKLEFCPIKTSFIENLIDNGDLYLFRINNKDFSSKSTGTK
NLHTLYLQAIFDERNLNNPTIMLNGGAELFYRKESIEQKNRITHKAGSILVNKVCKDGTS
LDDKIRNEIYQYENKFIDTLSDEAKKVLPNVIKKEATHDITKDKRFTSDKFFFHCPLTINY
KEGDTKQFNNEVLSFLRGNPDINIIGIDRGERNLIYVTVINQKGEILDSVSFNTVTNKSSKI
EQTVDYEEKLAVREKERIEAKRSWDSISKIATLKEGYLSAIVHEICLLMIKHNAIVVLENL
NAGFKRIRGGLSEKSVYQKFEKMLINKLNYFVSKKESDWNKPSGLLNGLQLSDQFESFE
KLGIQSGFIFYVPAAYTSKIDPTTGFANVLNLSKVRNVDAIKSFFSNFNEISYSKKEALFKF
SFDLDSLSKKGFSSFVKFSKSKWNVYTFGERIIKPKNKQGYREDKRINLTFEMKKLLNEY
KVSFDLENNLIPNLTSANLKDTFWKELFFIFKTTLQLRNSVTNGKEDVLISPVKNAKGEF
FVSGTHNKTLPQDCDANGAYHIALKGLMILERNNLVREEKDTKKIMAISNVDWFEYVQ
KRRGVL
SEQ ID NO: 51
MEHLETFNFFEEDRDRAEKYKILKEAIDEYHKKFIDEHLTNMSLDWNSLKQISEKYYKS
REEKDKKVFLSEQKRMRQEIVSEFKKDDRFKDLFSKKLFSELLKEEIYKKGNHQEIDALK
SFDKFSGYFIGLHENRKNMYSDGDEITAISNRIVNENFPKFLDNLQKYQEARKKYPEWII
KAESALVAHNIKMDEVFSLEYFNKVLNQEGIQRYNLALGGYVTKSGEKMMGLNDALN
LAHQSEKSSKGRIHMTPLFKQILSEKESFSYIPDVFTEDSQLLPSIGGFFAQIENDKDGNIF
DRALELISSYAEYDTERIYIRQADINRVSNVIFGEWGTLGGLMREYKADSINDINLERTCK
KVDKWLDSKEFALSDVLEAIKRTGNNDAFNEYISKMRTAREKIDAARKEMKFISEKISG
DEESIHIIKTLLDSVQQFLHFFNLFKARQDIPLDGAFYAEFDEVHSKLFAIVPLYNKVRNY
LTKNNLNTKKIKLNFKNPTLANGWDQNKVYDYASLIFLRDGNYYLGIINPKRKKNIKFE
QGSGNGPFYRKMVYKQIPGPNKNLPRVFLTSTKGKKEYKPSKEIIEGYEADKHIRGDKF
DLDFCHKLIDFFKESIEKHKDWSKFNFYFSPTESYGDISEFYLDVEKQGYRMHFENISAET
IDEYVEKGDLFLFQIYNKDFVKAATGKKDMHTIYWNAAFSPENLQDVVVKLNGEAELF
YRDKSDIKEIVHREGEILVNRTYNGRTPVPDKIHKKLTDYHNGRTKDLGEAKEYLDKVR
YFKAHYDITKDRRYLNDKIYFHVPLTLNFKANGKKNLNKMVIEKFLSDEKAHIIGIDRGE
RNLLYYSIIDRSGKIIDQQSLNVIDGFDYREKLNQREIEMKDARQSWNAIGKIKDLKEGY
LSKAVHEITKMAIQYNAIVVMEELNYGFKRGRFKVEKQIYQKFENMLIDKMNYLVFKD
APDESPGGVLNAYQLTNPLESFAKLGKQTGILFYVPAAYTSKIDPTTGFVNLFNTSSKTN
AQERKEFLQKFESISYSAKDGGIFAFAFDYRKFGTSKTDHKNVWTAYTNGERMRYIKEK
KRNELFDPSKEIKEALTSSGIKYDGGQNILPDILRSNNNGLIYTMYSSFIAAIQMRVYDGK
EDYIISPIKNSKGEFFRTDPKRRELPIDADANGAYNIALRGELTMRAIAEKFDPDSEKMAK
LELKHKDWFEFMQTRGD*
SEQ ID NO: 52
MHTGGLLSMDAKEFTGQYPLSKTLRFELRPIGRTWDNLEASGYLAEDRHRAECYPRAK
ELLDDNHRAFLNRVLPQIDMDWHPIAEAFCKVHKNPGNKELAQDYNLQLSKRRKEISA
YLQDADGYKGLFAKPALDEAMKIAKENGNESDIEVLEAFNGFSVYFTGYHESRENIYSD
EDMVSVAYRITEDNFPRFVSNALIFDKLNESHPDIISEVSGNLGVDDIGKYFDVSNYNNF
LSQAGIDDYNHIIGGHTTEDGLIQAFNVVLNLRHQKDPGFEKIQFKQLYKQILSVRTSKS
YIPKQFDNSKEMVDCICDYVSKIEKSETVERALKLVRNISSFDLRGIFVNKKNLRILSNKL
IGDWDAIETALMHSSSSENDKKSVYDSAEAFTLDDIFSSVKKFSDASAEDIGNRAEDICR
VISETAPFINDLRAVDLDSLNDDGYEAAVSKIRESLEPYMDLFHELEIFSVGDEFPKCAAF
YSELEEVSEQUEIIPLFNKARSFCTRKRYSTDKIKVNLKFPTLADGWDLNKERDNKAAIL
RKDGKYYLAILDMKKDLSSIRTSDEDESSFEKMEYKLLPSPVKMLPKIFVKSKAAKEKY
GLTDRMLECYDKGMHKSGSAFDLGFCHELIDYYKRCIAEYPGWDVFDFKFRETSDYGS
MKEFNEDVAGAGYYMSLRKIPCSEVYRLLDEKSIYLFQIYNKDYSENAHGNKNMHTMY
WEGLFSPQNLESPVFKLSGGAELFFRKSSIPNDAKTVHPKGSVLVPRNDVNGRRIPDSIY
RELTRYFNRGDCRISDEAKSYLDKVKTKKADHDIVKDRRFTVDKMMFHVPIAMNFKAI
SKPNLNKKVIDGIIDDQDLKIIGIDRGERNLIYVTMVDRKGNILYQDSLNILNGYDYRKA
LDVREYDNKEARRNWTKVEGIRKMKEGYLSLAVSKLADMIIENNAIIVMEDLNHGFKA
GRSKIEKQVYQKFESMLINKLGYMVLKDKSIDQSGGALHGYQLANHVTTLASVGKQCG
VIFYIPAAFTSKIDPTTGFADLFALSNVKNVASMREFFSKMKSVIYDKAEGKFAFTFDYL
DYNVKSECGRTLWTVYTVGERFTYSRVNREYVRKVPTDIIYDALQKAGISVEGDLRDRI
AESDGDTLKSIFYAFKYALDMRVENREEDYIQSPVKNASGEFFCSKNAGKSLPQDSDAN
GAYNIALKGILQLRMLSEQYDPNAESIRLPLITNKAWLTFMQSGMKTWKN
SEQ ID NO: 53
MDSLKDFTNLYPVSKTLRFELKPVGKTLENIEKAGILKEDEHRAESYRRVKKIIDTYHKV
FIDSSLENMAKMGIENEIKAMLQSFCELYKKDHRTEGEDKALDKIRAVLRGLIVGAFTG
VCGRRENTVQNEKYESLFKEKLIKEILPDFVLSTEAESLPFSVEEATRSLKEFDSFTSYFA
GFYENRKNIYSTKPQSTAIAYRLIHENLPKFIDNILVFQKIKEPIAKELEHIRADFSAGGYIK
KDERLEDIFSLNYYIHVLSQAGIEKYNALIGKIVTEGDGEMKGLNEHINLYNQQRGREDR
LPLFRPLYKQILSDREQLSYLPESFEKDEELLRALKEFYDHIAEDILGRTQQLMTSISEYDL
SRIYVRNDSQLTDISKKMLGDWNAIYMARERAYDHEQAPKRITAKYERDRIKALKGEES
ISLANLNSCIAFLDNVRDCRVDTYLSTLGQKEGPHGLSNLVENVFASYHEAEQLLSFPYP
EENNLIQDKDNVVLIKNLLDNISDLQRFLKPLWGMGDEPDKDERFYGEYNYIRGALDQ
VIPLYNKVRNYLTRKPYSTRKVKLNFGNSQLLSGWDRNKEKDNSCVILRKGQNFYLAI
MNNRHKRSFENKVLPEYKEGEPYFEKMDYKFLPDPNKMLPKVFLSKKGIEIYKPSPKLL
EQYGHGTHKKGDTFSMDDLHELIDFFKHSIEAHEDWKQFGFKFSDTATYENVSSFYREV
EDQGYKLSFRKVSESYVYSLIDQGKLYLFQIYNKDFSPCSKGTPNLHTLYWRMLFDERN
LADVIYKLDGKAEIFFREKSLKNDHPTHPAGKPIKKKSRQKKGEESLFEYDLVKDRHYT
MDKFQFHVPITMNFKCSAGSKVNDMVNAHIREAKDMHVIGIDRGERNLLYICVIDSRGT
ILDQISLNTINDIDYHDLLESRDKDRQQERRNWQTIEGIKELKQGYLSQAVHRIAELMVA
YKAVVALEDLNMGFKRGRQKVESSVYQQFEKQLIDKLNYLVDKKKRPEDIGGLLRAY
QFTAPFKSFKEMGKQNGFLFYIPAWNTSNIDPTTGFVNLFHAQYENVDKAKSFFQKFDSI
SYNPKKDWFEFAFDYKNFTKKAEGSRSMWILCTHGSRIKNFRNSQKNGQWDSEEFALT
EAFKSLFVRYEIDYTADLKTAIVDEKQKDFFVDLLKLFKLTVQMRNSWKEKDLDYLISP
VAGADGRFFDTREGNKSLPKDADANGAYNIALKGLWALRQIRQTSEGGKLKLAISNKE
WLQFVQERSYEKD
SEQ ID NO: 54
MTNKFTNQYSLSKTLRFELIPQGKTLEFIQEKGLLSQDKQRAESYQEMKKTIDKFHKYFI
DLALSNAKLTHLETYLELYNKSAETKKEQKFKDDLKKVQDNLRKEIVKSFSDGDAKSIF
AILDKKELITVELEKWFENNEQKDIYFDEKFKTFTTYFTGFHQNRKNMYSVEPNSTAIAY
RLIHENLPKFLENAKAFEKIKQVESLQVNFRELMGEFGDEGLIFVNELEEMFQINYYNDV
LSQNGITIYNSIISGFTKNDIKYKGLNEYINNYNQTKDKKDRLPKLKQLYKQILSDRISLSF
LPDAFTDGKQVLKAIFDFYKINLLSYTIEGQEESQNLLLLIRQTIENLSSFDTQKIYLKNDT
HLTTISQQVFGDFSVFSTALNYWYETKVNPKFETEYSKANEKKREILDKAKAVFTKQDY
FSIAFLQEVLSEYILTLDHTSDIVKKHSSNCIADYFKNHFVAKKENETDKTFDFIANITAK
YQCIQGILENADQYEDELKQDQKLIDNLKFFLDAILELLHFIKPLHLKSESITEKDTAFYD
VFENYYEALSLLTPLYNMVRNYVTQKPYSTEKIKLNFENAQLLNGWDANKEGDYLTTI
LKKDGNYFLAIMDKKHNKAFQKFPEGKENYEKMVYKLLPGVNKMLPKVFFSNKNIAY
FNPSKELLENYKKETHKKGDTFNLEHCHTLIDFFKDSLNKHEDWKYFDFQFSETKSYQD
LSGFYREVEHQGYKINFKNIDSEYIDGLVNEGKLFLFQIYSKDFSPFSKGKPNMHTLYWK
ALFEEQNLQNVIYKLNGQAEIFFRKASIKPKNIILHKKKIKIAKKHFIDKKTKTSEIVPVQT
IKNLNMYYQGKISEKELTQDDLRYIDNFSIFNEKNKTIDIIKDKRFTVDKFQFHVPITMNF
KATGGSYINQTVLEYLQNNPEVKIIGLDRGERHLVYLTLIDQQGNILKQESLNTITDSKIS
TPYHKLLDNKENERDLARKNWGTVENIKELKEGYISQVVHKIATLMLEENAIVVMEDL
NFGFKRGRFKVEKQIYQKLEKMLIDKLNYLVLKDKQPQELGGLYNALQLTNKFESFQK
MGKQSGFLFYVPAWNTSKIDPTTGFVNYFYTKYENVDKAKAFFEKFEAIRFNAEKKYFE
FEVKKYSDFNPKAEGTQQAWTICTYGERIETKRQKDQNNKFVSTPINLTEKIEDFLGKNQ
IVYGDGNCIKSQIASKDDKAFFETLLYWFKMTLQMRNSETRTDIDYLISPVMNDNGTFY
NSRDYEKLENPTLPKDADANGAYHIAKKGLMLLNKIDQADLTKKVDLSISNRDWLQFV
QKNK
SEQ ID NO: 55
MHENNGKIADNFIGIYPVSKTLRFELKPVGKTQEYIEKHGILDEDLKRAGDYKSVKKIID
AYHKYFIDEALNGIQLDGLKNYYELYEKKRDNNEEKEFQKIQMSLRKQIVKRFSEHPQY
KYLFKKELIKNVLPEFTKDNAEEQTLVKSFQEFTTYFEGFHQNRKNMYSDEEKSTAIAY
RVVHQNLPKYIDNMRIFSMILNTDIRSDLTELFNNLKTKMDITIVEEYFAIDGFNKVVNQ
KGIDVYNTILGAFSTDDNTKIKGLNEYINLYNQKNKAKLPKLKPLFKQILSDRDKISFIPE
QFDSDTEVLEAVDMFYNRLLQFVIENEGQITISKLLTNFSAYDLNKIYVKNDTTISAISND
LFDDWSYISKAVRENYDSENVDKNKRAAAYEEKKEKALSKIKMYSIEELNFFVKKYSC
NECHIEGYFERRILEILDKMRYAYESCKILHDKGLINNISLCQDRQAISELKDFLDSIKEVQ
WLLKPLMIGQEQADKEEAFYTELLRIWEELEPITLLYNKVRNYVTKKPYTLEKVKLNFY
KSTLLDGWDKNKEKDNLGIILLKDGQYYLGIMNRRNNKIADDAPLAKTDNVYRKMEY
KLLTKVSANLPRIFLKDKYNPSEEMLEKYEKGTHLKGENFCIDDCRELIDFFKKGIKQYE
DWGQFDFKFSDTESYDDISAFYKEVEHQGYKITFRDIDETYIDSLVNEGKLYLFQIYNKD
FSPYSKGTKNLHTLYWEMLFSQQNLQNIVYKLNGNAEIFYRKASINQKDVVVHKADLPI
KNKDPQNSKKESMFDYDIIKDKRFTCDKYQFHVPITMNFKALGENHFNRKVNRLIHDAE
NMHIIGIDRGERNLIYLCMIDMKGNIVKQISLNEIISYDKNKLEHKRNYHQLLKTREDEN
KSARQSWQTIHTIKELKEGYLSQVIHVITDLMVEYNAIVVLEDLNFGFKQGRQKFERQV
YQKFEKMLIDKLNYLVDKSKGMDEDGGLLHAYQLTDEFKSFKQLGKQSGFLYYIPAW
NTSKLDPTTGFVNLFYTKYESVEKSKEFINNFTSILYNQEREYFEFLFDYSAFTSKAEGSR
LKWTVCSKGERVETYRNPKKNNEWDTQKIDLTFELKKLFNDYSISLLDGDLREQMGKI
DKADFYKKFMKLFALIVQMRNSDEREDKLISPVLNKYGAFFETGKNERMPLDADANGA
YNIARKGLWIIEKIKNTDVEQLDKVKLTISNKEWLQYAQEHIL
SEQ ID NO: 56
MKQFTNLYQLSKTLRFELKPIGKTLEHINANGFIDNDAHRAESYKKVKKLIDDYHKDYI
ENVLNNFKLNGEYLQAYFDLYSQDTKDKQFKDIQDKLRKSIASALKGDDRYKTIDKKE
LIRQDMKTFLKKDTDKALLDEFYEFTTYFTGYHENRKNMYSDEAKSTAIAYRLIHDNLP
KFIDNIAVFKKIANTSVADNFSTIYKNFEEYLNVNSIDEIFSLDYYNIVLTQTQIEVYNSIIG
GRTLEDDTKIQGINEFVNLYNQQLANKKDRLPKLKPLFKQILSDRVQLSWLQEEFNTGA
DVLNAVKEYCTSYFDNVEESVKVLLTGISDYDLSKIYITNDLALTDVSQRMFGEWSIIPN
AIEQRLRSDNPKKTNEKEEKYSDRISKLKKLPKSYSLGYINECISELNGIDIADYYATLGAI
NTESKQEPSIPTSIQVHYNALKPILDTDYPREKNLSQDKLTVMQLKDLLDDFKALQHFIK
PLLGNGDEAEKDEKFYGELMQLWEVIDSITPLYNKVRNYCTRKPFSTEKIKVNFENAQL
LDGWDENKESTNASIILRKNGMYYLGIMKKEYRNILTKPMPSDGDCYDKVVYKFFKDIT
TMVPKCTTQMKSVKEHFSNSNDDYTLFEKDKFIAPVVITKEIFDLNNVLYNGVKKFQIG
YLNNTGDSFGYNHAVEIWKSFCLKFLKAYKSTSIYDFSSIEKNIGCYNDLNSFYGAVNLL
LYNLTYRKVSVDYIHQLVDEDKMYLFMIYNKDFSTYSKGTPNMHTLYWKMLFDESNL
NDVVYKLNGQAEVFYRKKSITYQHPTHPANKPIDNKNVNNPKKQSNFEYDLIKDKRYT
VDKFMFHVPITLNFKGMGNGDINMQVREYIKTTDDLHFIGIDRGERHLLYICVINGKGEI
VEQYSLNEIVNNYKGTEYKTDYHTLLSERDKKRKEERSSWQTIEGIKELKSGYLSQVIHK
ITQLMIKYNAIVLLEDLNMGFKRGRQKVESSVYQQFEKALIDKLNYLVDKNKDANEIGG
LLHAYQLTNDPKLPNKNSKQSGFLFYVPAWNTSKIDPVTGFVNLLDTRYENVAKAQAF
FKKFDSIRYNKEYDRFEFKFDYSNFTAKAEDTRTQWTLCTYGTRIETFRNAEKNSNWDS
REIDLTTEWKTLFTQHNIPLNANLKEAILLQANKNFYTDILHLMKLTLQMRNSVTGTDID
YMVSPVANECGEFFDSRKVKEGLPVNADANGAYNIARKGLWLAQQIKNANDLSDVKL
AITNKEWLQFAQKKQYLKD
SEQ ID NO: 57
MKQFTNQFSLSKTLRFELIPQGKTKEFIEINGLIEKDNERAVSYKKVKKIIDEYHKYFIEM
VLCDFKLHGLETYETIFNKKEKDDTDKKEFDNIRNSLRKQIADAFAKNPNDEIKERFKNL
FAKELIKQDLLNFVDDEQKELVNEFKDFTTYFTGFHQNRRNMYVADEKATAIAYRLVN
ENLPKFIDNLKIYEKIKKDAPELISDLNKTLVEMEEIVQGKTLDEIFSLSFFNQTLTQTGIE
LYNIVIGGRTADEGKTKIKGLNEYINTDYNQKQTDKKKKQAKFKQLYKQILSDRHSVSF
VAETFETDAQLLENIEQFYSSVLCNYEDDGHTTNIFEAIKNLIIGLKTFDLSKIYLRNDTSL
TDISQKLFGDWSIISSALNDYYEKQNPISSKEKQEKYDERKAKWLKQDFNIETIQTALNE
CDSEIIKEKNNKNIVSEYFAKLGLDKDNKIDLLQKIHHNYVVIKDLLNEPYPENIKLGNQ
KEQVSQIKDFLDSILNLIHFLKPLSLKDKDKEKDELFYSLFTALFEHLSQTISIYNKVRNYL
TQKAYSTEKIKLNFENSTLLNGWDVNKEPVNTSVIFRKNGLFYLGIMSKSNNRIFERNVP
VCKNEETAFEKMNYKLLPGANKMLPKVFLSAKGIESFQPSAEIQSKYQKETHKKGDAFV
RKDMENLIDFFKQSIAKHTDWKHFNHQFSKTETYNDLSEFYKEVEKQGYKLTFTKLDET
YINQLVDEGKLYLFQIYNKDFSPFSKGKPNMHTLYWKMLFDEQNLQNVVYKLNGEAE
VFFRQSSIKQTDRIIHKANQAIDNKNPLNNKKQSSFNYDLIKDKRFTLDKFQFHVPITLNF
KAEGNEYLNTKVNEYLKSNSDVKIIGLDRGERHLIYLTLINQKGELLKQQSLNVIATSQE
HETDYKNLLVNKENERANARQDWKTIETIKELKEGYLSQVVHQIATMMVDENAIVVM
EDLNAGFMRGRQKVERQVYQKLEKMLIEKLNYLVFKNNDVNETAGVLNALQLTNKFE
SFEKMGKQSGFLFYVPAWNTSKIDPATGFVDFLKPKYESVEKAKLFFEKFESIKFNADK
NYFEFEFDYKKFTEKAEGSQTKWTVCTHSDVRYRYNPQTKASDEVNVTNELKLIFDKF
KIEYKNGKNLKTELLLQDDKQLFSKLLHYLALTLMLRQSKSGTDIDFILSPVAKNGVFY
DSRNAMPNLPKDADANGAFHIALKGLWCVQQIKKADDLKKIKLAISNKEWLSFVQNLK
*EVMT*EAKLFQKALLL*TE*NMKKHQLEL
SEQ ID NO: 58
MYQKVKAILDDYHRDFIADMMGEVKLTKLAEFYDVYLKFRKNPKDDGLQKQLKDLQ
AVLRKEIVKPIGNGGKYKAGYDRLFGAKLFKDGKELGDLAKFVIAQEGESSPKLAHLAH
FEKFSTYFTGFHDNRKNMYSDEDKHTAIAYRLIHENLPRFIDNLQILATIKQKHSALYDQI
INELTASGLDVSLASHLDGYHKLLTQEGITAYNTLLGGISGEAGSRKIQGINELINSHHNQ
HCHKSERIAKLRPLHKQILSDGMGVSFLPSKFADDSEVCQAVNEFYRHYADVFAKVQSL
FDGFDDYQKDGIYVEYKNLNELSKQAFGDFALLGRVLDGYYVDVVNPEFNERFAKAKT
DNAKAKLTKEKDKFIKGVHSLASLEQAIEHYTARHDDESVQAGKLGQYFKHGLAGVD
NPIQKIHNNHSTIKGFLERERPAGERALPKIKSDKSPEIRQLKELLDNALNVAHFAKLLTT
KTTLHNQDGNFYGEFGALYDELAKIATLYNKVRDYLSQKPFSTEKYKLNFGNPTLLNG
WDLNKEKDNFGVILQKDGCYYLALLDKAHKKVFDNAPNTGKSVYQKMIYKLLPGPNK
MLPKVFFAKSNLDYYNPSAELLDKYAQGTHKKGDNFNLKDCHALIDFFKAGINKHPEW
QHFGFKFSPTSSYQDLSDFYREVEPQGYQVKFVDINADYINELVEQGQLYLFQIYNKDFS
PKAHGKPNLHTLYFKALFSEDNLVNPIYKLNGEAEIFYRKASLDMNETTIHRAGEVLEN
KNPDNPKKRQFVYDIIKDKRYTQDKFMLHVPITMNFGVQGMTIKEFNKKVNQSIQQYD
EVNVIGIDRGERHLLYLTVINSKGEILEQRSLNDITTASANGTQMTTPYHKILDKREIERL
NARVGWGEIETIKELKSGYLSHVVHQISQLMLKYNAIVVLEDLNFGFKRGRFKVEKQIY
QNFENALIKKLNHLVLKDKADDEIGSYKNALQLTNNFTDLKSIGKQTGFLFYVPAWNTS
KIDPETGFVDLLKPRYENIAQSQAFFGKFDKICYNADRGYFEFHIDYAKFNDKAKNSRQI
WKICSHGDKRYVYDKTANQNKGATIGVNVNDELKSLFTRYHINDKQPNLVMDICQNN
DKEFHKSLMYLLKTLLALRYSNASSDEDFILSPVANDEGVFFNSALADDTQPQNADANG
AYHIALKGLWLLNELKNSDDLNKVKLAIDNQTWLNFAQNR
SEQ ID NO: 59
MANSLKDFTNIYQLSKTLRFELKPIGKTEEHINRKLIIMHDEKRGEDYKSVTKLIDDYHR
KFIHETLDPAHFDWNPLAEALIQSGSKNNKALPAEQKEMREKIISMFTSQAVYKKLFKK
ELFSELLPEMIKSELVSDLEKQAQLDAVKSFDKFSTYFTGFHENRKNIYSKKDTSTSIAFR
IVHQNFPKFLANVRAYTLIKERAPEVIDKAQKELSGILGGKTLDDIFSIESFNNVLTQDKI
DYYNQIIGGVSGKAGDKKLRGVNEFSNLYRQQHPEVASLRIKMVPLYKQILSDRTTLSF
VPEALKDDEQAINAVDGLRSELERNDIFNRIKRLFGKNNLYSLDKIWIKNSSISAFSNELF
KNWSFIEDALKEFKENEFNGARSAGKKAEKWLKSKYFSFADIDAAVKSYSEQVSADISS
APSASYFAKFTNLIETAAENGRKFSYFAAESKAFRGDDGKTEIIKAYLDSLNDILHCLKPF
ETEDISDIDTEFYSAFAEIYDSVKDVIPVYNAVRNYTTQKPFSTEKFKLNFENPALAKGW
DKNKEQNNTAIILMKDGKYYLGVIDKNNKLRADDLADDGSAYGYMKMNYKFIPTPHM
ELPKVFLPKRAPKRYNPSREILLIKENKTFIKDKNFNRTDCHKLIDFFKDSINKHKDWRTF
GFDFSDTDSYEDISDFYMEVQDQGYKLTFTRLSAEKIDKWVEEGRLFLFQIYNKDFADG
AQGSPNLHTLYWKAIFSEENLKDVVLKLNGEAELFFRRKSIDKPAVHAKGSMKVNRRDI
DGNPIDEGTYVEICGYANGKRDMASLNAGARGLIESGLVRITEVKHELVKDKRYTIDKY
FFHVPFTINFKAQGQGNINSDVNLFLRNNKDVNIIGIDRGERNLVYVSLIDRDGHIKLQK
DFNIIGGMDYHAKLNQKEKERDTARKSWKTIGTIKELKEGYLSQVVHEIVRLAVDNNA
VIVMEDLNIGFKRGRFKVEKQVYQKFEKMLIDKLNYLVFKDAGYDAPCGILKGLQLTE
KFESFTKLGKQCGIIFYIPAGYTSKIDPTTGFVNLFNINDVSSKEKQKDFIGKLDSIRFDAK
RDMFTFEFDYDKFRTYQTSYRKKWAVWTNGKRIVREKDKDGKFRMNDRLLTEDMKNI
LNKYALAYKAGEDILPDVISRDKSLASEIFYVFKNTLQMRNSKRDTGEDFIISPVLNAKG
RFFDSRKTDAALPIDADANGAYHIALKGSLVLDAIDEKLKEDGRIDYKDMAVSNPKWFE
FMQTRKFDF
SEQ ID NO: 60
MRKFNEFVGLYPISKTLRFELKPIGKTLEHIQRNKLLEHDAVRADDYVKVKKIIDKYHKC
LIDEALSGFTFDTEADGRSNNSLSEYYLYYNLKKRNEQEQKTFKTIQNNLRKQIVNKLTQ
SEKYKRIDKKELITTDLPDFLTNESEKELVEKFKNFTTYFTEFHKNRKNMYSKEEKSTAI
AFRLINENLPKFVDNIAAFEKVVSSPLAEKINALYEDFKEYLNVEEISRVFRLDYYDELLT
QKQIDLYNAIVGGRTEEDNKIQIKGLNQYINEYNQQQTDRSNRLPKLKPLYKQILSDRES
VSWLPPKFDSDKNLLIKIKECYDALSEKEKVFDKLESILKSLSTYDLSKIYISNDSQLSYIS
QKMFGRWDIISKAIREDCAKRNPQKSRESLEKFAERIDKKLKTIDSISIGDVDECLAQLGE
TYVKRVEDYFVAMGESEIDDEQTDTTSFKKNIEGAYESVKELLNNADNITDNNLMQDK
GNVEKIKTLLDAIKDLQRFIKPLLGKGDEADKDGVFYGEFTSLWTKLDQVTPLYNMVR
NYLTSKPYSTKKIKLNFENSTLMDGWDLNKEPDNTTVIFCKDGLYYLGIMGKKYNRVF
VDREDLPHDGECYDKMEYKLLPGANKMLPKVFFSETGIQRFLPSEELLGKYERGTHKK
GAGFDLGDCRALIDFFKKSIERHDDWKKFDFKFSDTSTYQDISEFYREVEQQGYKMSFR
KVSVDYIKSLVEEGKLYLFQIYNKDFSAHSKGTPNMHTLYWKMLFDEENLKDVVYKLN
GEAEVFFRKSSITVQSPTHPANSPIKNKNKDNQKKESKFEYDLIKDRRYTVDKFLFHVPIT
MNFKSVGGSNINQLVKRHIRSATDLHIIGIDRGERHLLYLTVIDSRGNIKEQFSLNEIVNE
YNGNTYRTDYHELLDTREGERTEARRNWQTIQNIRELKEGYLSQVIHKISELAIKYNAVI
VLEDLNFGFMRSRQKVEKQVYQKFEKMLIDKLNYLVDKKKPVAETGGLLRAYQLTGE
FESFKTLGKQSGILFYVPAWNTSKIDPVTGFVNLFDTHYENIEKAKVFFDKFKSIRYNSD
KDWFEFVVDDYTRFSPKAEGTRRDWTICTQGKRIQICRNHQRNNEWEGQEIDLTKAFKE
HFEAYGVDISKDLREQINTQNKKEFFEELLRLLRLTLQMRNSMPSSDIDYLISPVANDTG
CFFDSRKQAELKENAVLPMNADANGAYNIARKGLLAIRKMKQEENDSAKISLAISNKE
WLKFAQTKPYLED
SEQ ID NO: 61
MSIYQEFVNKYSLSKTLRFELIPQGKTLENIKARGLILDDEKRAKDYKKAKQIIDKYHQF
FIEEILSSVCISEDLLQNYSDVYFKLKKSDDDNLQKDFKSAKDTIKKQISEYIKDSEKFKN
LFNQNLIDAKKGQESDLILWLKQSKDNGIELFKANSDITDIDEALEIIKSFKGWTTYFKGF
HENRKNVYSSNDIPTSIIYRIVDDNLPKFLENKAKYESLKDKAPEAINYEQIKKDLAEELT
FDIDYKTSEVNQRVFSLDEVFEIANFNNYLNQSGITKFNTIIGGKFVNGENTKRKGINEYI
NLYSQQINDKTLKKYKMSVLFKQILSDTESKSFVIDKLEDDSDVVTTMQSFYEQIAAFKT
VEEKSIKETLSLLFDDLKAQKLDLSKIYFKNDKSLTDLSQQVFDDYSVIGTAVLEYITQQI
APKNLDNPSKKEQELIAKKTEKAKYLSLETIKLALEEFNKHRDIDKQCRFEEILANFAAIP
MIFDEIAQNKDNLAQISIKYQNQGKKDLLQASAEDDVKAIKDLLDQTNNLLHKLKIFHIS
QSEDKANILDKDEHFYLVFEECYFELANIVPLYNKIRNYITQKPYSDEKFKLNFENSTLA
NGWDKNKEPDNTAILFIKDDKYYLGVMNKKNNKIFDDKAIKENKGEGYKKIVYKLLPG
ANKMLPKVFFSAKSIKFYNPSEDILRIRNHSTHTKNGSPQKGYEKFEFNIEDCRKFIDFYK
QSISKHPEWKDFGFRFSDTQRYNSIDEFYREVENQGYKLTFENISESYIDSVVNQGKLYL
FQIYNKDFSAYSKGRPNLHTLYWKALFDERNLQDVVYKLNGEAELFYRKQSIPKKITHP
AKEAIANKNKDNPKKESVFEYDLIKDKRFTEDKFFFHCPITINFKSSGANKFNDEINLLLK
EKANDVHILSIDRGERHLAYYTLVDGKGNIIKQDTFNIIGNDRMKTNYHDKLAAIEKDR
DSARKDWKKINNIKEMKEGYLSQVVHEIAKLVIEYNAIVVFEDLNFGFKRGRFKVEKQV
YQKLEKMLIEKLNYLVFKDNEFDKTGGVLRAYQLTAPFETFKKMGKQTGITYYVPAGFT
SKICPVTGFVNQLYPKYESVSKSQEFFSKFDKICYNLDKGYFEFSFDYKNFGDKAAKGK
WTIASFGSRLINFRNSDKNHNWDTREVYPTKELEKLLKDYSIEYGHGECIKAAICGESDK
KFFAKLTSVLNTILQMRNSKTGTELDYLISPVADVNGNFFDSRQAPKNMPQDADANGA
YHIGLKGLMLLGRIKNNQEGKKLNLVIKNEEYFEFVQNRNN
SEQ ID NO: 62
MEDYSGFVNIYSIQKTLRFELKPVGKTLEHIEKKGFLKKDKIRAEDYKAVKKIIDKYHRA
YIEEVFDSVLHQKKKKDKTRFSTQFIKEIKEFSELYYKTEKNIPDKERLEALSEKLRKML
VGAFKGEFSEEVAEKYKNLFSKELIRNEIEKFCETDEERKQVSNFKSFTTYFTGFHSNRQ
NIYSDEKKSTAIGYRIIHQNLPKFLDNLKIIESIQRRFKDFPWSDLKKNLKKIDKNIKLTEY
FSIDGFVNVLNQKGIDAYNTILGGKSEESGEKIQGLNEYINLYRQKNNIDRKNLPNVKILF
KQILGDRETKSFIPEAFPDDQSVLNSITEFAKYLKLDKKKKSIIAELKKFLSSFNRYELDGI
YLANDNSLASISTFLFDDWSFIKKSVSFKYDESVGDPKKKIKSPLKYEKEKEKWLKQKY
YTISFLNDAIESYSKSQDEKRVKIRLEAYFAEFKSKDDAKKQFDLLERIEEAYAIVEPLLG
AEYPRDRNLKADKKEVGKIKDFLDSIKSLQFFLKPLLSAEIFDEKDLGFYNQLEGYYEEI
DSIGHLYNKVRNYLTGKIYSKEKFKLNFENSTLLKGWDENREVANLCVIFREDQKYYLG
VMDKENNTILSDIPKVKPNELFYEKMVYKLIPTPHMQLPRIIFSSDNLSIYNPSKSILKIRE
AKSFKEGKNFKLKDCHKFIDFYKESISKNEDWSRFDFKFSKTSSYENISEFYREVERQGY
NLDFKKVSKFYIDSLVEDGKLYLFQIYNKDFSIFSKGKPNLHTIYFRSLFSKENLKDVCLK
LNGEAEMFFRKKSINYDEKKKREGHHPELFEKLKYPILKDKRYSEDKFQFHLPISLNFKS
KERLNFNLKVNEFLKRNKDINIIGIDRGERNLLYLVMINQKGEILKQTLLDSMQSGKGRP
EINYKEKLQEKEIERDKARKSWGTVENIKELKEGYLSIVIHQISKLMVENNAIVVLEDLNI
GFKRGRQKVERQVYQKFEKMLIDKLNFLVFKENKPTEPGGVLKAYQLTDEFQSFEKLS
KQTGFLFYVPSWNTSKIDPRTGFIDFLHPAYENIEKAKQWINKFDSIRFNSKMDWFEFTA
DTRKFSENLMLGKNRVWVICTTNVERYFTSKTANSSIQYNSIQITEKLKELFVDIPFSNGQ
DLKPEILRKNDAVFFKSLLFYIKTTLSLRQNNGKKGEEEKDFILSPVVDSKGRFFNSLEAS
DDEPKDADANGAYHIALKGLMNLLVLNETKEENLSRPKWKIKNKDWLEFVWERNR
SEQ ID NO: 63
MSRPYNISLDIGTSSIGWSVVDDQSKLVSVRGKYGYGVRLYDEGQTAAERRSFRTTRRR
LKRRKWRLGLLREIFEPYITPVDDTFFLRQKQSNLSPKDQRKLYPQTSLFNDRTDRAFYD
DYPTIYHLRYKLMTEKRQFDIREIYLAMHHIVKYRGHFLNEAPVSSFKSSEINLVAHFDR
LNTIFADLFSESGFQLETDKLAEVKALLLDNQQSASNRQRQALSLIYTPSTNKAVEKQNK
AIATELLKAILGLKAKFNVLTGIEAEDVKAWTLTFNAENFDEEMVKLESSLDDNAHQIIE
SLQELYSGVLLAGIVPENQSLSQAMITKYDDHQKHLKMLKAVREALAPEDRQRLKQAY
DQYVDGQENTKAYSKEDFYGDITKALKNNPDHPIVSEIKKLIELDQFMPKQRTKDNGAI
PHQLHQQELDRIIENQQQYYPWLAELNPNSKRQTVAKYKLDELVAFRVPYYVGPLITAE
QQQQSSDAKFAWMIRKAEGRITPWNFDDKVDRQASANEFIKRMTTTDTYLLAEDVLPK
QSLIYQRFEVLNELNGLKIDDQPITTELKQAIFTDLFMQKISVTVKNIQDYLVSEKRYASR
PAITGLSDENKFNSRLSTYHDLKAIVGDAVEDVDKQVDLEKCVEWSTIFEDGKIYSAKL
NEIDWLTDQQRVQLAAKRYRGWGRLSAKLLTQIVNANGQRIMDLLWDTTDNFMRIVH
SEDFDKLITEANQMMLAENDVQDVINDLYTSPQNKKALRQILLVVNDIQKAMKGQAPE
RILIEFAREDEVNRRLSVQRKRQVEQVYQNISNELLNNTEIRNELKDLSNSALSNTRLFLY
FMQGGRDMYTGDSLNIDRLSTYDIDHILPQSFIKDNSLDNRVLVSQKMNRSKADQVPTD
FTSVELGKKMQLQWEQMLRAGLITKKKYDNLTLNPDHISKYAMKGFINRQLVETRQVI
KLATNLLMEQYGEDNIELITVKSGLTHQMRTEFDFPKNRNLNNHHHAFDAYLTAFVGL
YLLKRYPKLKPYFVYGEYQKASQQDKWRNFNFLNGLKKDELVDENTEAVIWDKESGL
AYLNKIYQFKKILVTREVHENSGALFNQTLYAAKDDKASGQGSKQLIPAKQNRPTALYG
GYSGKTVAYMCIVRIKNKKGDLYKVCGVETSWLAQLKQLTDEDSQKAFLKQKISPQFT
KVKKQKGTIVKVVEDFEVIAPHILINQRFFDNGQELTLGSATYKHNEQELILDKTAVKLL
NGALPLTQSEELAEQVYDEILDQVMHYFPLYDTNQFRAKLSAGKAAFMKLPWKSQWD
GNKMVQVGQQVILDRVLIGLHANAAVSDLGILKLTTPFGKLQKSSGIYLSPDTQIIYQSP
TGLFERRVALRDL
SEQ ID NO: 64
MTKREEPYNVGLDIGSSSVGWAVVDNNYHLLNIKKNNLWGARLFKEAETAQVTRGHR
SMRRRYRRRRNRLNWLDELFADELAKIDPSFLLRMKNSWVSKKESTRKRDPYNLFIDE
KYNDVDYFNQYPTIFHLRKELITEDKKVDIRLIYLAIHNIIKYRGNFTLENQNFDISQLSSN
FSQQISDFFALFSDFGVIMPEDFDPDKISDILLNPNLSPSGKVSEAIATISPKTNVKAKIKIIL
LLLVGNNGDLKKLFDLETTEKIAVKLSSRHIDSELPIILSELNEQQENIITIANSIFGSIILKD
FLGDETSISAAKVISFEDHKQDLQKLKTMWRETSNKEAVKAGKKAYEDYIGHEDSETFY
KKIKKFLEKAQPVDLANKALAEIELENYLPKQRNRNNTVIPYQLNENELIAILDHQEKYY
PFLKENRDKILSLLTFRIPYYVGPLQDSNNNRFSWMTRKASGAIRPWNFSEKVNVEQSSN
DFIKRMRSTDTYLIGEPVLPKKSLIYQCYEVLSELNNTRVKDGSSNPKRLDVTIKQRIYNE
IFKNQKSVSVKVLQNWLIKESYFKSPEISGLADKKKYLSSLSTYIDFKKIFGQRFVDDPVN
SPQLEELAEWLTLFEDKKILLIKLQNSKYSYDQATINKLSTMRYQGTGKLSKKLLVDLK
TTKKSIGKSGAESLSILDLMWSTKDNFMQIIHDADYTFEQQIKEFNYDTEDELTPLEKVA
NLHGSPALKRGLNQSIKVVADIVKFMGHDPEKIFIEFTRSDDFSKLTISRYRRIKKQYLEI
AKAIKKIPAEFKDIKEYQTQLEENKGKLASERLMLYFLQCGHSLYSNKPIDLNMINSSKY
HVDHILPQSYIKDDSLENKALVLASENENKIDNMLISHDIIATNLPRWQALKDQNLMGS
KKFADLTRTTVTENQKKGFIQRQLVQTSQIVKNITLILNDLYKNTSCIETRATLSSEFRKA
FSNFDETTYHYQFPEFVKNRDVNDFHHAQDAFLACVIGEYQLKKYPKDNLRLVYDQYS
KFLDSLKKDTRKKNGRMPRYTQNGFIIGSMFNGKTYVDDNGEIIWDQKIKESIRKTFNY
HQFNVVRQTIEQHGKLFNDTIQPHSDRYKLIPLKTNRDPAIYGGYNNDNNAYSVVLDVD
GKKKINGIPIRIANQLKSDELDLSSWLENNIKHKKPMTILIDKVPKYQRIINEETGDLLITS
ANEVINNVQLFLPSMYTALISLLDSTKTEMYSKLLSNYEANILIDIYDYLLTKLKNNYPL
YRKEWAKLAEHRDDFIESDLVTQASTLQQLIKFMHADPSNVNLKFGNFKGNRFGRKNG
NIKLSKTDFIYESPTGLFKSIKHID
SEQ ID NO: 65
MTKEYYLGLDVGTNSVGWAVTDSQYNLCKFKKKDMWGIRLFESANTAKDRRLQRGN
RRRLERKKQRIDLLQEIFSPEICKIDPTFFIRLNESRLHLEDKSNDFKYPLFIEKDYSDIEYY
KEFPTIFHLRKHLIESEEKQDIRLIYLALHNIIKTRGHFLIDGDLQSAKQLRPILDTFLLSLQ
EEQNLSVSLSENQKDEYEEILKNRSIAKSEKVKKLKNLFEISDELEKEEKKAQSAVIENFC
KFIVGNKGDVCKFLRVSKEELEIDSFSFSEGKYEDDIVKNLEEKVPEKVYLFEQMKAMY
DWNILVDILETEEYISFAKVKQYEKHKTNLRLLRDIILKYCTKDEYNRMFNDEKEAGSY
TAYVGKLKKNNKKYWIEKKRNPEEFYKSLGKLLDKIEPLKEDLEVLTMMIEECKNHTL
LPIQKNKDNGVIPHQVHEVELKKILENAKKYYSFLTETDKDGYSVVQKIESIFRFRIPYYV
GPLSTRHQEKGSNVWMVRKPGREDRIYPWNMEEIIDFEKSNENFITRMTNKCTYLIGED
VLPKHSLLYSKYMVLNELNNVKVRGKKLPTSLKQKVFEDLFENKSKVTGKNLLEYLQI
QDKDIQIDDLSGFDKDFKTSLKSYLDFKKQIFGEEIEKESIQNMIEDIIKWITIYGNDKEML
KRVIRANYSNQLTEEQMKKITGFQYSGWGNFSKMFLKGISGSDVSTGETFDIITAMWET
DNNLMQILSKKFTFMDNVEDFNSGKVGKIDKITYDSTVKEMFLSPENKRAVWQTIQVA
EEIKKVMGCEPKKIFIEMARGGEKVKKRTKSRKAQLLELYAACEEDCRELIKEIEDRDER
DFNSMKLFLYYTQFGKCMYSGDDIDINELIRGNSKWDRDHIYPQSKIKDDSIDNLVLVN
KTYNAKKSNELLSEDIQKKMHSFWLSLLNKKLITKSKYDRLTRKGDFTDEELSGFIARQ
LVETRQSTKAIADIFKQIYSSEVVYVKSSLVSDFRKKPLNYLKSRRVNDYHHAKDAYLNI
VVGNVYNKKFTSNPIQWMKKNRDTNYSLNKVFEHDVVINGEVIWEKCTYHEDTNTYD
GGTLDRIRKIVERDNILYTEYAYCEKGELFNATIQNKNGNSTVSLKKGLDVKKYGGYFS
ANTSYFSLIEFEDKKGDRARHIIGVPIYIANMLEHSPSAFLEYCEQKGYQNVRILVEKIKK
NSLLIINGYPLRIRGENEVDTSFKRAIQLKLDQKNYELVRNIEKFLEKYVEKKGNYPIDEN
RDHITHEKMNQLYEVLLSKMKKFNKKGMADPSDRIEKSKPKFIKLEDLIDKINVINKML
NLLRCDNDTKADLSLIELPKNAGSFVVKKNTIGKSKIILVNQSVTGLYENRREL
SEQ ID NO: 66
MQTLFENFTNQYPVSKTLRFELIPQGKTKDFIEQKGLLKKDEDRAEKYKKVKNIIDEYH
KDFIEKSLNGLKLDGLEEYKTLYLKQEKDDKDKKAFDKEKENLRKQIANAFRNNEKFK
TLFAKELIKNDLMSFACEEDKKNVKEFEAFTTYFTGFHQNRANMYVADEKRTAIASRLI
HENLPKFIDNIKIFEKMKKEAPELLSPFNQTLKDMKDVIKGTTLEEIFSLDYFNKTLTQSGI
DIYNSVIGGRTPEEGKTKIKGLNEYINTDFNQKQTDKKKRQPKFKQLYKQILSDRQSLSFI
AEAFKNDTEILEAIEKFYVNELLHFSNEGKSTNVLDAIKNAVSNLESFNLTKIYFRSGTSL
TDVSRKVFGEWSIINRALDNYYATTYPIKPREKSEKYEERKEKWLKQDFNVSLIQTAIDE
YDNETVKGKNSGKVIVDYFAKFCDDKETDLIQKVNEGYIAVKDLLNTPYPENEKLGSN
KDQVKQIKAFMDSIMDIMHFVRPLSLKDTDKEKDETFYSLFTPLYDHLTQTIALYNKVR
NYLTQKPYSTEKIKLNFENSTLLGGWDLNKETDNTAIILRKENLYYLGIMDKRHNRIFRN
VPKADKKDSCYEKMVYKLLPGANKMLPKVFFSQSRIQEFTPSAKLLENYENETHKKGD
NFNLNHCHQLIDFFKDSINKHEDWKNFDFRFSATSTYADLSGFYHEVEHQGYKISFQSIA
DSFIDDLVNEGKLYLFQIYNKDFSPFSKGKPNLHTLYWKMLFDENNLKDVVYKLNGEA
EVFYRKKSIAEKNTTIHKANESIINKNPDNPKATSTFNYDIVKDKRYTIDKFQFHVPITMN
FKAEGIFNMNQRVNQFLKANPDINIIGIDRGERHLLYYTLINQKGKILKQDTLNVIANEK
QKVDYHNLLDKKEGDRATARQEWGVIETIKELKEGYLSQVIHKLTDLMIENNAIIVMED
LNFGFKRGRQKVEKQVYQKFEKMLIDKLNYLVDKNKKANELGGLLNAFQLANKFESF
QKMGKQNGFIFYVPAWNTSKTDPATGFIDFLKPRYENLKQAKDFFEKFDSIRLNSKADY
FEFAFDFKNFTGKADGGRTKWTVCTTNEDRYAWNRALNNNRGSQEKYDITAELKSLFD
GKVDYKSGKDLKQQIASQELADFFRTLMKYLSVTLSLRHNNGEKGETEQDYILSPVADS
MGKFFDSRKAGDDMPKNADANGAYHIALKGLWCLEQISKTDDLKKVKLAISNKEWLE
FMQTLKG
SEQ ID NO: 67
AAGCATTGGCCGTAAGTGCGATTCCGGAAAGGAGATATACATGCACCATCATCATC
ACCATTCATCGCTCACGAAATTCACTAACAAATACTCTAAACAGCTCACCATTAAGA
ATGAACTCATCCCAGTTGGCAAAACACTGGAGAACATCAAAGAGAATGGTCTGATA
GATGGCGACGAACAGCTGAATGAGAATTATCAGAAGGCGAAAATTATTGTGGATGA
TTTTCTGCGGGACTTCATTAATAAAGCACTGAATAATACGCAGATCGGGAACTGGCG
CGAACTGGCGGATGCCCTTAATAAAGAGGATGAAGATAACATCGAGAAATTGCAGG
ATAAAATTCGGGGAATCATTGTATCCAAATTTGAAACGTTTGATCTGTTTAGCAGCT
ATTCTATTAAGAAAGATGAAAAGATTATTGACGACGACAATGATGTTGAAGAAGAG
GAACTGGATCTGGGCAAGAAGACCAGCTCATTTAAATACATATTTAAAAAAAACCT
GTTTAAGTTAGTGTTGCCATCCTACCTGAAAACCACAAACCAGGACAAGCTGAAGA
TTATTAGCTCGTTTGATAATTTTTCAACGTACTTCCGCGGGTTCTTTGAAAACCGGAA
AAACATTTTTACCAAGAAACCGATCTCCACAAGTATTGCGTATCGCATTGTTCATGA
TAACTTCCCGAAATTCCTTGATAACATTCGTTGTTTTAATGTGTGGCAGACGGAATG
CCCGCAACTAATCGTGAAAGCAGATAACTATCTGAAAAGCAAAAATGTTATAGCGA
AAGATAAAAGTTTGGCAAACTATTTTACCGTGGGCGCGTATGACTATTTCCTGTCTC
AGAATGGTATAGATTTTTACAACAATATTATAGGTGGACTGCCAGCGTTCGCCGGCC
ATGAGAAAATCCAAGGTCTCAATGAATTCATCAATCAAGAGTGCCAAAAAGACAGC
GAGCTGAAAAGTAAGCTGAAAAACCGTCACGCGTTCAAAATGGCGGTACTGTTCAA
ACAGATACTCAGCGATCGTGAAAAAAGTTTTGTAATTGATGAGTTCGAGTCGGATGC
TCAAGTTATTGACGCCGTTAAAAACTTTTACGCCGAACAGTGCAAAGATAACAATGT
TATTTTTAACTTATTAAATCTTATCAAGAATATCGCTTTCTTAAGTGATGACGAACTG
GACGGCATATTCATTGAAGGGAAATACCTGTCGAGCGTTAGTCAAAAACTCTATAG
CGATTGGTCAAAATTACGTAACGACATTGAGGATTCGGCTAACTCTAAACAAGGCA
ATAAAGAGCTGGCCAAGAAGATCAAAACCAACAAAGGGGATGTAGAAAAAGCGAT
CTCGAAATATGAGTTCTCGCTGTCGGAACTGAACTCGATTGTACATGATAACACCAA
GTTTTCTGACCTCCTTAGTTGTACACTGCATAAGGTGGCTTCTGAGAAACTGGTGAA
GGTCAATGAAGGCGACTGGCCGAAACATCTCAAGAATAATGAAGAGAAACAAAAA
ATCAAAGAGCCGCTTGATGCTCTGCTGGAGATCTATAATACACTTCTGATTTTTAAC
TGCAAAAGCTTCAATAAAAACGGCAACTTCTATGTCGACTATGATCGTTGCATCAAT
GAACTGAGTTCGGTCGTGTATCTGTATAATAAAACACGTAACTATTGCACTAAAAAA
CCCTATAACACGGACAAGTTCAAACTCAATTTTAACAGTCCGCAGCTCGGTGAAGGC
TTTTCCAAGTCGAAAGAAAATGACTGTCTGACTCTTTTGTTTAAAAAAGACGACAAC
TATTATGTAGGCATTATCCGCAAAGGTGCAAAAATCAATTTTGATGATACACAAGCA
ATCGCCGATAACACCGACAATTGCATCTTTAAAATGAATTATTTCCTACTTAAAGAC
GCAAAAAAATTTATCCCGAAATGTAGCATTCAGCTGAAAGAAGTCAAGGCCCATTT
TAAGAAATCTGAAGATGATTACATTTTGTCTGATAAAGAGAAATTTGCTAGCCCGCT
GGTCATTAAAAAGAGCACATTTTTGCTGGCAACTGCACATGTGAAAGGGAAAAAAG
GCAATATCAAGAAATTTCAGAAAGAATATTCGAAAGAAAACCCCACTGAGTATCGC
AATTCTTTAAACGAATGGATTGCTTTTTGTAAAGAGTTCTTAAAAACTTATAAAGCG
GCTACCATTTTTGATATAACCACATTGAAAAAGGCAGAGGAATATGCTGATATTGTA
GAATTCTACAAGGAT
SEQ ID NO: 68
AAGCATTGGCCGTAAGTGCGATTCCGGAAAGGAGATATACATGCACCATCATCATC
ACCATAACAACTACGACGAATTCACCAAACTGTACCCGATCCAGAAAACCATCCGT
TTCGAACTGAAACCGCAGGGTCGTACCATGGAACACCTGGAAACCTTCAACTTCTTC
GAAGAAGACCGTGACCGTGCGGAAAAATACAAAATCCTGAAAGAAGCGATCGACG
AATACCACAAAAAATTCATCGACGAACACCTGACCAACATGTCTCTGGACTGGAAC
TCTCTGAAACAGATCTCTGAAAAATACTACAAATCTCGTGAAGAAAAAGACAAAAA
AGTTTTCCTGTCTGAACAGAAACGTATGCGTCAGGAAATCGTTTCTGAATTCAAAAA
AGACGACCGTTTCAAAGACCTGTTCTCTAAAAAACTGTTCTCTGAACTGCTGAAAGA
AGAAATCTACAAAAAAGGTAACCACCAGGAAATCGACGCGCTGAAATCTTTCGACA
AATTCTCTGGTTACTTCATCGGTCTGCACGAAAACCGTAAAAACATGTACTCTGACG
GTGACGAAATCACCGCGATCTCTAACCGTATCGTTAACGAAAACTTCCCGAAATTCC
TGGACAACCTGCAGAAATACCAGGAAGCGCGTAAAAAATACCCGGAATGGATCATC
AAAGCGGAATCTGCGCTGGTTGCGCACAACATCAAAATGGACGAAGTTTTCTCTCTG
GAATACTTCAACAAAGTTCTGAACCAGGAAGGTATCCAGCGTTACAACCTGGCGCT
GGGTGGTTACGTTACCAAATCTGGTGAAAAAATGATGGGTCTGAACGACGCGCTGA
ACCTGCTCGCACCAGTCTGAAAAATCTTCTAAAGGTCGTATCCACATGACCCCGCTGT
TCAAACAGATCCTGTCTGAAAAAGAATCTTTCTCTTACATCCCGGACGTTTTCACCG
AAGACTCTCAGCTGCTGCCGTCTATCGGTGGTTTCTTCGCGCAGATCGAAAACGACA
AAGACGGTAACATCTTCGACCGTGCGCTGGAACTGATCTCTTCTTACGCGGAATACG
ACACCGAACGTATCTACATCCGTCAGGCGGACATCAACCGTGTTTCTAACGTTATCT
TCGGTGAATGGGGTACCCTGGGTGGTCTGATGCGTGAATACAAAGCGGACTCTATC
AACGACATCAACCTGGAACGTACCTGCAAAAAAGTTGACAAATGGCTGGACTCTAA
AGAATTCGCGCTGTCTGACGTTCTCTGAAGCGATCAAACGTACCGGTAACAACGACG
CGTTCAACGAATACATCTCTAAAATGCGTACCGCGCGTGAAAAAATCGACGCGGCG
CGTAAAGAAATGAAATTCATCTCTGAAAAAATCTCTGGTGACGAAGAATCTATCCA
CATCATCAAAACCCTGCTGGACTCTGTTCAGCAGTTCCTCTCACTTCTTCAACCTGTTC
AAAGCGCGTCAGGACATCCCGCTGGACGGTGCGTTCTACGCGGAATTCGACGAAGT
TCACTCTAAACTGTTCGCGATCGTTCCGCTGTACAACAAAGTTCGTAACTACCTGAC
CAAAAACAACCTGAACACCAAAAAAATCAAACTGAACTTCAAAAACCCGACCCTGG
CGAACGGTTGGGACCAGAACAAAGTTTACGACTACGCGTCTCTGATCTTCCTGCGTG
ACGGTAACTACTACCTGGGTATCATCAACCCGAAACGTAAAAAAAACATCAAATTC
GAACACTGGTTCTGGTAACGGTCCGTTCTACCGTAAAATGGTTTACAAACAGATCCCG
GGTCCGAACAAAAACCTGCCGCGTGTTTTCCTGACCTCTACCAAAGGTAAAAAAGA
ATACAAACCGTCTAAAGAAATCATCGAAGGTTACGAAGCGGACAAACACATCCGTG
GTGACAAATTCGACCTGGACTTCTGCCACAAACTGATCGACTTCTTCAAAGAATCTA
TCGAAAAACACAAAGACTGGTCTAAATTCAACTTCTACTTCTCTCCGACCGAATCTT
ACGGTGACATCTCTGAATTCTACCTCTGAC
SEQ ID NO: 69
ACTAAAACATTTGATTCAGAGTTTTTTAATTTGTACTCGCTGCAAAAAACGGTACGC
TTTGAGTTAAAACCCGTGGGAGAAACCGCGTCATTTGTGGAAGACTTTAAAAACGA
GGGCTTGAAACGTGTTGTGAGCGAAGATGAAAGGCGAGCCGTCGATTACCAGAAAG
TTAAGGAAATAATTGACGATTACCATCGGGATTTCATTGAAGAAAGTTTAAATTATT
TTCCGGAACAGGTGAGTAAAGATGCTCTTGAGCAGGCGTTTCATCTTTATCAGAAAC
TGAAGGCAGCAAAAGTTGAGGAAAGGGAAAAAGCGCTGAAAGAATGGGAAGCGCT
GCAGAAAAAGCTACGTGAAAAAGTGGTGAAATGCTTCTCGGACTCGAATAAAGCCC
GCTTCTCAAGGATTGATAAAAAGGAACTGATTAAGGAAGACCTGATAAATTGGTTG
GTCGCCCAGAATCGCGAGGATGATATCCCTACGGTCGAAACGTTTAACAACTTCACC
ACATATTTTACCGGCTTCCATGAGAATCGTAAAAATATTTACTCCAAAGATGATCAC
GCCACCGCTATTAGCTTTCGCCTTATTCATGAAAATCTTCCAAAGTTTTTTGACAACG
TGATTAGCTTCAATAAGTTGAAAGAGGGTTTCCCTGAATTAAAATTTGATAAAGTGA
AAGAGGATTTAGAAGTAGATTATGATCTGAAGCATGCGTTTGAAATAGAATATTTCG
TTAACTTCGTGACCCAAGCGGGCATAGATCACTATAATTATCTGTTAGGAGGGAAA
ACCCTGGAGGACGGGACGAAAAAACAAGGGATGAATGAGCAAATTAATCTGTTCAA
ACAACAGCAAACGCGAGATAAAGCGCGTCAGATTCCCAAACTGATCCCCCTGTTCA
AACAGATTCTTAGCGAAAGGACTGAAAGCCAGTCCTTTATTCCTAAACAATTTGAAA
GTGATCAGGAGTTGTTCGATTCACTGCAGAAGTTACATAATAACTGCCAGGATAAAT
TCACCGTGCTGCAACAAGCCATTCTCGGTCTGGCAGAGGCGGATCTTAAGAAGGTCT
TCATCAAAACCTCTGATTTAAATGCCTTATCTAACACCATTTTCGGGAATTACAGCG
TCTTTTCCGATGCACTGAACCTGTATAAAGAAAGCCTGAAAACGAAAAAAGCGCAG
GAGGCTTTTGAGAAACTACCGGCCCATTCTATTCACGACCTCATTCAATACTTGGAA
CAGTTCAATTCCAGCCTGGACGCGGAAAAACAACAGAGCACCGACACCGTCCTGAA
CTACTTCATCAAGACCGATGAATTATATTCTCGCTTCATTAAATCCACTAGCGAGGC
TTTCACTCAGGTGCAGCCTTTGTTCGAACTGGAAGCCCTGTCATCTAAGCGCCGCCC
ACCGGAATCGGAAGATGAAGGGGCAAAAGGGCAGGAAGGCTTCGAGCAGATCAAG
CGTATTAAAGCTTACCTGGATACGCTTATGGAAGCGGTACACTTTGCAAAGCCGTTG
TATCTTGTTAAGGGTCGTAAAATGATCGAAGGGCTCGATAAAGACCAGTCCTTTTAT
GAAGCGTTTGAAATGGCGTACCAAGAACTTGAATCGTTAATCATTCCTATCTATAAC
AAAGCGCGGAGCTATCTGTCGCGGAAACCTTTCAAGGCCGATAAATTCAAGATTAA
TTTTGACAACAACACGCTACTGAGCGGATGGGATGCGAACAAGGAAACTGCTAACG
CGTCCATTCTGTTTAAGAAAGACGGGTTATATTACCTTGGAATTATGCCGAAAGGTA
AGACCTTTCTCTTTGACTACTTTGTATCGAGCGAGGATTCAGAGAAACTGAAACAGC
GTCGCCAGAAGACCGCCGAAGAAGCTCTGGCGCAGGATGGTGAAAGTTAC
SEQ ID NO: 70
AAGCATTGGCCGTAAGTGCGATTCCGGAAAGGAGATATACATGCACCATCATCATC
ACCATCATACAGGCGGTCTTCTTAGTATGGACGCGAAAGAGTTCACAGGTCAGTATC
CGTTGTCGAAAACATTACGATTCGAACTTCGGCCCATCGGCCGCACGTGGGATAACC
TGGAGGCCTCAGGCTACTTAGCGGAAGACCGCCATCGTGCCGAATGTTATCCTCGTG
CGAAAGAGTTATTGGATGACAACCATCGTGCCTTCCTGAATCGTGTGTTGCCACAAA
TCGATATGGATTGGCACCCGATTGCGGAGGCCTTTTGTAAGGTACATAAAAACCCTG
GTAATAAAGAACTTGCCCAGGATTACAACCTTCAGTTGTCAAAGCGCCGTAAGGAG
ATCAGCGCATATCTTCAGGATGCAGATGGCTATAAAGGCCTGTTCGCGAAGCCCGCC
TTAGACGAAGCTATGAAAATTGCGAAAGAAAACGGGAACGAAAGTGATATTGAGGT
TCTCGAAGCGTTTAACGGTTTTAGCGTATACTTCACCGGTTATCATGAGTCACGCGA
GAACATTTATAGCGATGAGGATATGGTGAGCGTAGCCTACCGAATTACTGAGGATA
ATTTCCCGCGCTTTGTCTCAAACGCTTTGATCTTTGATAAATTAAACGAAAGCCATCC
GGATATTATCTCTGAAGTATCGGGCAATCTTGGAGTTGATGACATTGGTAAGTACTT
TGACGTGTCGAACTATAACAATTTTCTTTCCCAGGCCGGTATAGATGACTACAATCA
CATTATTGGCGGCCATACAACCGAAGACGGACTGATACAAGCGTTTAATGTCGTATT
GAACTTACGTCACCAAAAAGACCCTGGCTTTGAAAAAATTCAGTTCAAACAGCTCTA
CAAACAAATCCTGAGCGTGCGTACCAGCAAAAGCTACATCCCGAAACAGTTTGACA
ACTCTAAGGAGATGGTTGACTGCATTTGCGATTATGTCAGCAAAATAGAGAAATCC
GAAACAGTAGAACGGGCCCTGAAACTAGTCCGTAATATCAGTTCTTTCGACTTGCGC
GGGATCTTTGTCAATAAAAAGAACTTGCGCATACTGAGCAACAAACTGATAGGAGA
TTGGGACGCGATCGAAACCGCATTGATGCATAGTTCTTCATCAGAAAACGATAAGA
AAAGCGTATATGATAGCGCGGAGGCTTTTACGTTGGATGACATCTTTTCAAGCGTGA
AAAAATTTTCTGATGCCTCTGCCGAAGATATTGGCAACAGGGCGGAAGACATCTGT
AGAGTGATAAGTGAGACGGCCCCTTTTATCAACGATCTGCGAGCGGTGGACCTGGA
TAGCCTGAACGACGATGGTTATGAAGCGGCCGTCTCAAAAATTCGGGAGTCGCTGG
AGCCTTATATGGATCTTTTCCATGAACTGGAAATTTTCTCGGTTGGCGATGAGTTCCC
AAAATGCGCAGCATTTTACAGCGAACTGGAGGAAGTCAGCGAACAGCTGATCGAAA
TTATTCCGTTATTCAACAAGGCGCGTTCGTTCTGCACCCGGAAACGCTATAGCACCG
ATAAGATTAAAGTGAACTTAAAATTCCCGACCTTGGCGGACGGGTGGGACCTGAAC
AAAGAGAGAGACAACAAAGCCGCGATTCTGCGGAAAGACGGTAAGTATTATCTGGC
AATTCTGGATATGAAGAAAGATCTGTCAAGCATTAGGACCAGCGACGAAGATGAAT
CCAGCTTCGAAAAGATGGAGTATAAACTGTTACCGAGTCCAGTAAAAATGCTGCCA
AAGATATTCGTAAAATCGAAAGCCGCTAAGGAAAAATATGGCCTGACAGATCGTAT
GCTTGAATGCTACGATAAAGGTATGCATAAGTCGGGTAGTGCGTTTGATCTTGGCTT
TTGCCATGAACTCATTGATTATTACAAGCGTTGTATCGCGGAGTACCCAGGCTGGGA
TGTGTTCGATTTCAAGTTTCGCGAAACTTCCGATTATGGGTCCATGAAAGAGTTCAA
TGAAGAT
SEQ ID NO: 71
GATAGTTTGAAAGATTTCACCAATCTGTACCCTGTCAGTAAGACATTGAGATTTGAA
TTAAAGCCCGTTGGAAAGACTTTAGAAAATATCGAGAAAGCAGGTATTTTGAAAGA
GGATGAGCATCGTGCAGAAACSTTATCGGAGGGTGAAGAAAATAATTGATACTTATC
ATAAGGTATTTATCGATTCTTCTCTTGAAAATATGGCTAAAATGGGTATTGAGAATG
AAATAAAAGCAATGCTCCAAAGTTTCTGCGAATTGTATAAAAAAGATCATCGCACT
GAGGGTGAAGACAAGGCATTAGATAAAATTCGAGCAGTACTTCGTGGCCTGATTGT
TGGGGCTTTCACTGGTGTTTGCGGAACACGGGAAAATACAGTCCAAAACGAGAAGT
ACGAGAGTTTGTTCAAAGAAAAGTTGATAAAAGAAATTTTACCTGATTTTGTGCTCT
CTACTGAGGCTGAAAGCTTGCCTTTCTCTGTTGAAGAAGCTACGAGGTCACTGAAGG
AGTTTGATAGCTTTACATCCTACTTTGCTGGTTTTTACGAGAATAGAAAGAATATAT
ACTCGACGAAACCTCAATCCACTGCCATTGCTTATCGTCTTATTCATGAGAACTTGC
CGAAGTTCATTGATAATATTCTTGTTTTTCAGAAGATCAAAGAGCCTATAGCCAAAG
AGCTGGAACATATTCGTGCGGACTTTTCTGCCGGGGGGTACATAAAAAAGGATGAG
AGATTGGAGGATATTTTTTCGTTGAACTATTATATCCACGTGTTATCTCAGGCTGGG
ATCGAAAAATATAACGCATTGATTGGGAAGATTGTGACAGAAGGAGATGGAGAGAT
GAAAGGGCTCAATGAACACATCAACCTTTACAACCAACAAAGAGGCAGAGAGGATC
GGCTCCCTCTTTTTAGGCCTCTTTATAAACAGATATTGAGTGACAGAGAGCAATTAT
CATACTTGCCTGAGAGTTTTGAAAAAGATGAGGAGCTCCTCAGGGCTCTAAAAGAG
TTCTATGATCATATCGCAGAAGACATTCTCGGACGTACTCAACAGTTGATGACTTCT
ATTTCAGAATATGATTTATCTCGGATATACGTAAGGAACGATAGCCAATTGACTGAT
ATATCAAAAAAAATGTTGGGAGATTGGAATGCTATCTACATGGCTAGAGAACGAGC
ATATGACCACGAGCAGGCTCCCAAAAGAATCACGGCGAAATACGAGAGGGACAGG
ATTAAAGCTCTTAAAGGAGAAGAGAGTATAAGTCTGGCAAATCTTAATAGTTGTATT
GCCTTTCTGGACAATGTTAGAGATTGTCGTGTAGATACTTATCTTTCCACACTGGGC
CAGAAGGAAGGACCACATGGTCTATCTAATCTCGTTGAGAACGTTTTTGCCTCATAC
CATGAAGCAGAGCAATTGTTGAGCTTTCCATACCCCGAAGAGAATAATCTGATTCAG
GACAAGGACAATGTGGTGTTAATTAAGAATCTTCTCGACAATATCAGTGATCTGCAG
AGGTTCTTGAAACCTCTTTGGGGTATGGGAGACGAACCCGATAAAGATGAAAGATT
TTATGGAGAGTATAATTATATCCGAGGAGCTCTAGATCAGGTGATCCCTCTGTACAA
TAAGGTAAGGAACTACCTCACTCGGAAGCCTTATTCGACCAGAAAAGTAAAACTCA
ATTTTGGGAATTCTCAATTGCTTAGTGGTTGGGATAGAAATAAGGAAAAGGATAAT
AGCTGTGTGATTTTGCGTAAGGGGCAGAACTTCTATTTGGCTATTATGAACAATAGG
CACAAAAGAAGTTTCGAAAACAAGGTGTTGCCCGAGTATAAGGAGGGAGAACCTTA
C
SEQ ID NO: 72
AAGCATTGGCCGTAAGTGCGATTCCGGAAAGGAGATATACATGAACAACGGCACAA
ATAATTTTCAGAACTTCATCGGGATCTCAAGTTTGCAGAAAACGCTGCGCAATGCTC
TGATCCCCACGGAAACCACGCAACAGTTCATCGTCAAGAACGGAATAATTAAAGAA
GATGAGTTACGTGGCGAGAACCGCCAGATTCTGAAAGATATCATGGATGACTACTA
CCGCGGATTCATCTCTGAGACTCTGAGTTCTATTGATGACATAGATTGGACTAGCCT
GTTCGAAAAAATGGAAATTCAGCTGAAAAATGGTGATAATAAAGATACCTTAATTA
AGGAACAGACAGAGTATCGGAAAGCAATCCATAAAAAATTTGCGAACGACGATCG
GTTTAAGAACATGTTTAGCGCCAAACTGATTAGTGACATATTACCTGAATTTGTCAT
CCACAACAATAATTATTCGGCATCAGAGAAAGAGGAAAAAACCCAGGTGATAAAAT
TGTTTTCGCGCTTTGCGACTAGCTTTAAAGATTACTTCAAGAACCGTGCAAATTGCTT
TTCAGCGGACGATATTTCATCAAGCAGCTGCCATCGCATCGTCAACGACAATGCAGA
GATATTCTTTTCAAATGCGCTGGTCTACCGCCGGATCGTAAAATCGCTGAGCAATGA
CGATATCAACAAAATTTCGGGCGATATGAAAGATTCATTAAAAGAAATGAGTCTGG
AAGAAATATATTCTTACGAGAAGTATGGGGAATTTATTACCCAGGAAGGCATTAGC
TTCTATAATGATATCTGTGGGAAAGTGAATTCTTTTATGAACCTGTATTGTCAGAAA
AATAAAGAAAACAAAAATTTATACAAACTTCAGAAACTTCACAAACAGATTCTATG
CATTGCGGACACTAGCTATGAGGTCCCGTATAAATTTGAAAGTGACGAGGAAGTGT
ACCAATCAGTTAACGGCTTCCTTGATAACATTAGCAGCAAACATATAGTCGAAAGAT
TACGCAAAATCGGCGATAACTATAACGGCTACAACCTGGATAAAATTTATATCGTGT
CCAAATTTTACGAGAGCGTTAGCCAAAAAACCTACCGCGACTGGGAAACAATTAAT
ACCGCCCTCGAAATTCATTACAATAATATCTTGCCGGGTAACGGTAAAAGTAAAGCC
GACAAAGTAAAAAAAGCGGTTAAGAATGATTTACAGAAATCCATCACCGAAATAAA
TGAACTAGTGTCAAACTATAAGCTGTGCAGTGACGACAACATCAAAGCGGAGACTT
ATATACATGAGATTAGCCATATCTTGAATAACTTTGAAGCACAGGAATTGAAATACA
ATCCGGAAATTCACCTAGTTGAATCCGAGCTCAAAGCGAGTGAGCTTAAAAACGTG
CTGGACGTGATCATGAATGCGTTTCATTGGTGTTCGGTTTTTATGACTGAGGAACTT
GTTGATAAAGACAACAATTTTTATGCGGAACTGGAGGAGATTTACGATGAAATTTAT
CCAGTAATTAGTCTGTACAACCTGGTTCGTAACTACGTTACCCAGAAACCGTACAGC
ACGAAAAAGATTAAATTGAACTTTGGAATACCGACGTTAGCAGACGGTTGGTCAAA
GTCCAAAGAGTATTCTAATAACGCTATCATACTGATGCGCGACAATCTGTATTATCT
GGGCATCTTTAATGCGAAGAATAAACCGGACAAGAAGATTATCGAGGGTAATACGT
CAGAAAATAAGGGTGACTACAAAAAGATGATTTATAATTTGCTCCCGGGTCCCAAC
AAAATGATCCCGAAAGTTTTCTTGAGCAGCAAGACGGGGGTGGAAACGTATAAACC
GAGCGCCTATATCCTAGAGGGGTATAAACAGAATAAACATATCAAGTCTTCAAAAG
ACTTTGATATCACTTTCTGTCATGATCTGATCGACTACTTCAAAAACTGTATTGCAAT
TCATCCCGAGTGGAAAAACTTCGGTTTTGATTTTAGCGACACCAGTACTTATGAAGA
CATTTCCGGGTTTTATCGTGAGGTAGAGTTACAAGGTTACAAGATTGATTGGACATA
CATTA
SEQ ID NO: 73
ACCAATAAATTCACTAACCAGTATTCTCTCTCTAAGACCCTGCGCTTTGAACTGATTC
CGCAGGGGAAAACCTTGGAGTTCATTCAAGAAAAAGGCCTCTTGTCTCAGGATAAA
CAGAGGGCTGAATCTTACCAAGAAATGAAGAAAACTATTGATAAGTTTCATAAATA
TTTCATTGATTTAGCCTTGTCTAACGCCAAATTAACTCACTTGGAAACGTATCTGGA
GTTATACAACAAATCTGCCGAAACTAAGAAAGAACAGAAATTTAAAGACGATTTGA
AAAAAGTACAGGACAATCTGCGTAAAGAAATTGTCAAATCCTTCAGTGACGGCGAT
GCTAAAAGCATTTTTGCCATTCTGGACAAAAAAGAGTTGATTACTGTGGAATTAGAA
AAGTGGTTTGAAAACAATGAGCAGAAAGACATCTACTTCGATGAGAAATTCAAAAC
TTTCACCACCTATTTTACAGGATTTCATCAAAACCGGAAGAACNTGTACTCAGTAGA
ACCGAACTCCACGGCCATTGCGTATCGTTTGATCCATGAGAATCTGCCTAAATTTCT
GGAGAATGCGAAAGCCTTTGAAAAGATTAAGCAGGTCGAATCGCTGCAAGTGAATT
TTCGTGAACTCATGGGCGAATTTGGTGACGAAGGTCTAATCTTCGTTAACGAACTGG
AAGAAATGTTTCAGATTAATTACTACAATGACGTGCTATCGCAGAACGGTATCACAA
TCTACAATAGTATTATCTCAGGGTTCACAAAAAACGATATAAAATACAAAGGCCTG
AACGAGTATATCAATAACTACAACCAAACAAAGGACAAAAAGGATAGGCTTCCGAA
ACTGAAGCAGTTATACAAACAGATTTTATCTGACAGAATCTCCCTGAGCTTTCTGCC
GGATGCTTTCACTGATGGGAAGCAGGTTCTGAAAGCGATTTTCGATTTTTATAAGAT
TAACTTACTGAGCTACACGATTGAAGGTCAAGAAGAATCTCAAAACTTACTGCTCTT
GATCCGTCAAACCATTGAAAATCTATCATCGTTCGATACGCAGAAAATCTACCTCAA
AAACGATACTCACCTGACTACGATCTCTCAGCAGGITTTCGGGGATTTTAGTGTATT
TTCAACAGCTCTGAACTACTGGTATGAAACCAAAGTCAATCCGAAATTCGAGACGG
AATATTCTAAGGCCAACGAAAAAAAACGTGAGATTCTTGATAAAGCTAAAGCCGTA
TTTACTAAACAGGATTACTTTTCTATTGCTTTCCTGCAGGAAGTTTTATCGGAGTATA
TCCTGACCCTGGATCATACATCTGATATCGTTAAAAAACACAGCAGCAATTGCATCG
CTGACTATTTCAAAAACCACTTTGTCGCCAAAAAAGAAAACGAAACAGACAAGACT
TTCGATTTCATTGCTAACATCACCGCAAAATACCAGTGTATTCAGGGTATCTTGGAA
AACGCCGACCAATACGAAGACGAACTGAAACAAGATCAGAAGCTGATCGATAATTT
AAAATTCTTCTTAGATGCAATCCTGGAGCTGCTGCACTTCATCAAACCGCTTCATTTA
AAGAGCGAGTCCATTACCGAAAAGGACACCGCCTTCTATGACGTTTTTGAAAATTAT
TATGAAGCCCTCTCCTTGCTGACTCCGCTGTATAATATGGTACGCAATTACGTAACC
CAGAAACCATATTCTACCGAAAAAATTAAACTGAACTTTGAAAACGCACAGCTGCT
CAACGGTTGGGACGCGAATAAAGAAGGTGACTACCTCACCACCATCCTGAAAAAAG
ATGGTAACTATTTTCTGGCAATTATGGATAAGAAACATAATAAAGCATTCCAGAAAT
TTCCTGAAGGGAAAGAAAAT
SEQ ID NO: 74
AAGCATTGGCCGTAAGTGCGATTCCGGAAAGGAGATATACATGCACCATCATCATC
ACCATTCTTTCGACTCTTTCACCAACCTGTACTCTCTGTCTAAAACCCTGAAATTCGA
AATGCGTCCGGTTGGTAACACCCAGAAAATGCTGGACAACGCGGGTGTTTTCGAAA
AAGACAAACTGATCCAGAAAAAATACGGTAAAACCAAACCGTACTTCGACCGTCTG
CACCGTGAATTCATCGAAGAAGCGCTGACCGGTGTTGAACTGATCGGTCTGGACGA
AAACTTCCGTACCCTGGTTGACTGGCAGAAAGACAAAAAAAACAACGTTGCGATGA
AAGCGTACGAAAACTCTCTGCAGCGTCTGCGTACCGAAATCGGTAAAATCTTCAACC
TGAAAGCGGAAGACTGGGTTAAAAACAAATACCCGATCCTGGGTCTGAAAAACAAA
AACACCGACATCCTGTTCGAAGAAGCGGTTTTCGGTATCCTGAAAGCGCGTTACGGT
GAAGAAAAAGACACCTTCATCGAAGTTGAAGAAATCGACAAAACCGGTAAATCTAA
AATCAACCAGATCTCTATCTTCGACTCTTGGAAAGGTTTCACCGGTTACTTCAAAAA
ATTCTTCGAAACCCGTAAAAACTTCTACAAAAACGACGGTACCTCTACCGCGATCGC
GACCCGTATCATCGACCAGAACCTGAAACGTTTCATCGACAACCTGTCTATCGTTGA
ATCTGTTCGTCAGAAAGTTGACCTGGCGGAAACCGAAAAATCTTTCTCTATCTCTCT
GTCTCAGTTCTTCTCTATCGACTTCTACAACAAATGCCTGCTGCAGGACGGTATCGA
CTACTACAACAAAATCATCGGTGGTGAAACCCTGAAAAACGGTGAAAAACTGATCG
GTCTGAACGAACTGATCAACCAGTACCGTCAGAACAACAAAGACCAGAAAATCCCG
TTCTTCAAACTGCTGGACAAACAGATCCTGTCTGAAAAAATCCTGTTCCTGGACGAA
ATCAAAAACGACACCGAACTGATCGAAGCGCTGTCTCAGTTCGCGAAAACCGCGGA
AGAAAAAACCAAAATCGTTAAAAAACTGTTCGCGGACTTCGTTGAAAACAACTCTA
AATACGACCTGGCGCAGATCTACATCTCTCAGGAAGCGTTCAACACCATCTCTAACA
AATGGACCTCTGAAACCGAAACCTTCGCGAAATACCTGTTCGAAGCGATGAAATCT
GGTAAACTGGCGAAATACGAAAAAAAAGACAACTCTTACAAATTCCCGGACTTCAT
CGCGCTGTCTCAGATGAAATCTGCGCTGCTGTCTATCTCTCTGGAAGGTCACTTCTG
GAAAGAAAAATACTACAAAATCTCTAAATTCCAGGAAAAAACCAACTGGGAACAGT
TCCTGGCGATCTTCCTGTACGAATTCAACTCTCTGTTCTCTGACAAAATCAACACCA
AAGACGGTGAAACCAAACAGGTTGGTTACTACCTGTTCGCGAAAGACCTGCACAAC
CTGATCCTGTCTGAACAGATCGACATCCCGAAAGACTCTAAAGTTACCATCAAAGAC
TTCGCGGACTCTGTTCTGACCATCTACCAGATGGCGAAATACTTCGCGGTTGAAAAA
AAACGTGCGTGGCTGGCGGAATACGAACTGGACTCTTTCTACACCCAGCCGGACAC
CGGTTACCTGCAGTTCTACGACAACGCGTACGAAGACATCGTTCAGGTTTACAACAA
ACTGCGTAACTACCTGACCAAAAAACCGTACTCTGAAGAAAAATGGAAACTGAACT
TCGAAAACTCTACCCTGGCGAACGGTTGGGACAAAAACAAAGAATCTGACAACTCT
GCGGTTATCCTGCAGAAAGGTGGTAAATACTACCTGGGTCTGATCACCAAAGGTCA
CAACAAAATCTTCGACGACCGTTTCCAGGAAAAATTCATCGTTGGTATCGAAGGTGG
TAAATACGAAAAAATCGTTTACAAATTCTTCCCGGACCAGGCGAAAATGTTCCCGA
AAGTTTGCTTCTCTGCGAAAGGTCTGGAATTCTTCCGTCCGTCTGAAGAAATCCTGC
GTATCTACAACAACGCGGAATTCAAAAAAGGTGAAACCTACTCTATCGACTCTATGC
AGAAACTGATCGACTTCTACAAAGACTGCCTGACCAAATACGAAGGTTGGGCGTGC
TACACCTTCCGTCACCTGAAACCGACCGAAGAATACCAGAACAACATCGGTGAATT
CTTCCGTGAC
SEQ ID NO: 75
ACCCAGTTCGAAGGTTTCACCAACCTGTACCAGGTTTCTAAAACCCTGCGTTTCGAA
CTGATCCCGCAGGGTAAAACCCTGAAACACATCCAGGAACAGGGTTTCATCGAAGA
AGACAAAGCGCGTAACGACCACTACAAAGAACTGAAACCGATCATCGACCGTATCT
ACAAAACCTACGCGGACCAGTGCCTGCAGCTGGTTCAGCTGGACTGGGAAAACCTG
TCTGCGGCGATCGACTCTTACCGTAAAGAAAAAACCGAAGAAACCCGTAACGCGCT
GATCGAAGAACAGGCGACCTACCGTAACGCGATCCACGACTACTTCATCGGTCGTA
CCGACAACCTGACCGACGCGATCAACAAACGTCACGCGGAAATCTACAAAGGTCTG
TTCAAAGCGGAACTGTTCAACGGTAAAGTTCTGAAACAGCTGGGTACCGTTACCACC
ACCGAACACGAAAACGCGCTGCTGCGTTCTTTCGACAAATTCACCACCTACTTCTCT
GGTTTCTACGAAAACCGTAAAAACGTTTTCTCTGCGGAAGACATCTCTACCGCGATC
CCGCACCGTATCGTTCAGGACAACTTCCCGAAATTCAAAGAAAACTGCCACATCTTC
ACCCGTCTGATCACCGCGGTTCCGTCTCTGCGTGAACACTTCGAAAACGTTAAAAAA
GCGATCGGTATCTTCGTTTCTACCTCTATCGAAGAAGTTTTCTCTTTCCCGTTCTACA
ACCAGCTGCTGACCCAGACCCAGATCGACCTGTACAACCAGCTGCTGGGTGGTATCT
CTCGTGAAGCGGGTACCGAAAAAATCAAAGGTCTGAACGAAGTTCTGAACCTGGCG
ATCCAGAAAAACGACGAAACCGCGCACATCATCGCGTCTCTGCCGCACCGTTTCATC
CCGCTGTTCAAACAGATCCTGTCTGACCGTAACACCCTGTCTTTCATCCTGGAAGAA
TTCAAATCTGACGAAGAAGTTATCCAGTCTTTCTGCAAATACAAAACCCTGCTGCGT
AACGAAAACGTTCTGGAAACCGCGGAAGCGCTGTTC
SEQ ID NO: 76
GTCGATAATCTGTGCTACAAACTGGAGTTCTGCCCGATTAAAACCTCGTTTATAGAA
AACCTGATAGATAACGGCGACCTGTATCTGTTTCGCATCAATAACAAAGACTTCAGC
AGTAAATCGACCGGCACCAAGAACCTTCATACGTTATATTTACAAGCTATATTCGAT
GAACGTAATCTGAACAATCCGACAATTATGCTGAATGGGGGAGCAGAACTGTTCTA
TCGTAAAGAAAGTATTGAGCAGAAAAACCGTATCACACACAAAGCCGGTTCAATTC
TCGTGAATAAGGTGTGTAAAGACGGTACAAGCCTGGATGATAAGATACGTAATGAA
ATTTATCAATATGAGAATAAATTTATTGATACCCTGTCTGATGAAGCTAAAAAGGTG
TTACCGAATGTCATTAAAAAGGAAGCTACCCATGACATTACAAAAGATAAACGTTT
CACTAGTGACAAATTCTTCTTTCACTGCCCCCTGACAATTAATTATAAGGAAGGCGA
TACCAAGCAGTTCAATAACGAAGTGCTGAGTTTTCTGCGTGGAAATCCTGACATCAA
CATTATCGGCATTGACCGCGGAGAGCGTAATTTAATCTATGTAACGGTTATAAACCA
GAAAGGCGAGATTCTGGATTCGGTTTCATTCAATACCGTGACCAACAAGAGTTCAA
AAATCGAGCAGACAGTCGATTATGAAGAGAAATTGGCAGTCCGCGAGAAAGAGAG
GATTGAAGCAAAACGTTCCTGGGACTCTATCTCAAAAATTGCGACACTAAAGGAAG
GTTATCTGAGCGCAATAGTTCACGAGATCTGTCTGTTAATGATTAAACACAACGCGA
TCGTTGTCTTAGAGAATCTTAATGCAGGCTTTAAGCGTATTCGTGGCGGTTTATCAG
AAAAAAGTGTTTATCAAAAATTCGAAAAAATGTTGATTAACAAACTGAACTATTTTG
TCAGCAAGAAGGAATCCGACTGGAATAAACCGTCTGGTCTGCTGAATGGACTGCAG
CTTTCGGATCAGTTTGAAAGCTTCGAAAAACTGGGTATTCAGTCTGGTTTTATTTTTT
ACGTGCCGGCTGCATATACCTCA
SEQ ID NO: 77
AAGATTGATCCGACCACGGGCTTCGCCAATGTTCTGAATCTGTCGAAGGTACGCAAT
GTTGATGCGATCAAAAGCTTTTTTTCTAACTTCAACGAAATTAGTTATAGCAAGAAA
GAAGCCCTTTTCAAATTCTCATTCGATCTGGATTCACTGAGTAAGAAAGGCTTTAGT
AGCTTTGTGAAATTTAGTAAGAGTAAATGGAACGTCTACACCTTTGGAGAACGTATC
ATAAAGCCAAAGAATAAGCAAGGTTATCGGGAGGACAAAAGAATCAACTTGACCTT
CGAGATGAAGAAGTTACTTAACGAGTATAAGGTTTCTTTTGATCTTGAAAATAACTT
GATTCCGAATCTCACGAGTGCCAACCTGAAGGATACTTTTTGGAAAGAGCTATTCTT
TATCTTCAAGACTACGCTGCAGCTCCGTAACAGCGTTACTAACGGTAAAGAAGATGT
GCTCATCTCTCCGGTCAAAAATGCGAAGGGTGAATTCTTCGTTTCGGGAACGCATAA
CAAGACTCTTCCGCAAGATTGCGATGCGAACGGTGCATACCATATTGCGTTGAAAG
GTCTGATGATACTCGAACGTAACAACCTTGTACGTGAGGAGAAAGATACGAAAAAG
ATTATGGCGATTTCAAACGTGGATTGGTTCGAGTACGTGCAGAAACGTAGAGGCGTT
CTGTAAGAAATCATCCTTAGCGAAAGCTAAGGATTTTTTTTATCTGAAATGTAGGGA
GACCCTCAGGTTAAATATTCACTCAGGAAGTTA
SEQ ID NO: 78
AAAATCGACCCGACCACCGGTTTCGTTAACCTGTTCAACACCTCTTCTAAAACCAAC
GCGCAGGAACGTAAAGAATTCCTGCAGAAATTCGAATCTATCTCTTACTCTGCGAAA
GACGGTGGTATCTTCGCGTTCGCGTTCGACTACCGTAAATTCGGTACCTCTAAAACC
GACCACAAAAACGTTTGGACCGCGTACACCAACGGTGAACGTATGCGTTACATCAA
AGAAAAAAAACGTAACGAACTGTTCGACCCGTCTAAAGAAATCAAAGAAGCGCTGA
CCTCTTCTGGTATCAAATACGACGGTGGTCAGAACATCCTGCCGGACATCCTGCGTT
CTAACAACAACGGTCTGATCTACACCATGTACTCTTCTTTCATCGCGGCGATCCAGA
TGCGTGTTTACGACGGTAAAGAAGACTACATCATCTCTCCGATCAAAAACTCTAAAG
GTGAATTCTTCCGTACCGACCCGAAACGTCGTGAACTGCCGATCGACGCGGACGCG
AACGGTGCGTACAACATCGCGCTGCGTGGTGAACTGACCATGCGTGCGATCGCGGA
AAAATTCGACCCGGACTCTGAAAAAATGGCGAAACTGGAACTGAAACACAAAGACT
GGTTCGAATTCATGCAGACCCGTGGTGACTAAGAAATCATCCTTAGCGAAAGCTAA
GGATTTTTTTTATCTGAAATGTAGGGAGACCCTCAGGTTAAATATTCACTCAGGAAG
TTA
SEQ ID NO: 79
GTGCGGCTGCATTTTTTATGTGCCTGCTGCATACACGAGCTTCTGTTTTACGTGCCGG
CAGATTATACTTCAAAAATCGATCCAACAACTGGCTTTGTGAACTTCCTGGACCTGA
GATATCAGTCTGTAGAAAAAGCTAAACAACTTCTTAGCGATTTTAATGCCATTCGTT
TTAACAGCGTTCAGAATTACTTTGAATTCGAAATTGACTATAAAAAACTTACTCCGA
AACGTAAAGTCGGAACCCAAAGTAAATGGGTAATTTGTACGTATGGCGATGTCAGG
TATCAGAACCGTCGGAATCAAAAAGGTCATTGGGAGACCGAAGAAGTGAACGTGAC
CGAAAAGCTGAAGGCTCTGTTCGCCAGCGATTCAAAAACTACAACTGTGATCGATT
ACGCAAATGATGATAACCTGATAGATGTGATTTTAGAGCAGGATAAAGCCAGCTTTT
TTAAAGAACTGTTGTGGCTCCTGAAACTTACGATGACCTTACGACATTCCAAGATCA
AATCGGAAGATGATTTTATTCTGTCACCGGTCAAGAATGAGCAGGGTGAATTCTATG
ATAGTAGGAAAGCCGGCGAAGTGTGGCCGAAAGACGCCGACGCCAATGGCGCCTAT
CATATCGCGCTCAAAGGGCTTTGGAATTTGCAGCAGATTAACCAGTGGGAAAAAGG
TAAAACCCTGAATCTGGCTATCAAAAACCAGGATTGGTTTAGCTTTATCCAAGAGAA
ACCGTATCAGGAATGAGAAATCATCCTTAGCGAAAGCTAAGGATTTTTTTTATCTGA
AATGTAGGGAGACCCTCAGGTTAAATATTCACTCAGGAAGTTA
SEQ ID NO: 80
GGTTATCTTTTATATACCGGCAGCGTTCACTAGTAAAATAGATCCGACCACTGGTTT
CGCCGATCTCTTTGCCCTGAGTAACGTTAAAAACGTAGCGAGCATGCGTGAATTCTT
TTCCAAAATGAAATCTGTCATTTATGATAAAGCTGAAGGCAAATTCGCATTCACCTT
TGATTACTTGGATTACAACGTGAAGAGCGAATGTGGTCGTACGCTGTGGACCGTTTA
CACCGTTGGTGAGCGCTTCACCTATTCCCGTGTGAACCGCGAATATGTACGTAAAGT
CCCCACCGATATTATCTATGATGCCCTCCAGAAAGCAGGCATTAGCGTCGAAGGAG
ACTTAAGGGACAGAATTGCCGAAAGCGATGGCGATACGCTGAAGTCTATTTTTTACG
CATTCAAATACGCGCTAGATATGCGCGTTGAGAATCGCGAGGAAGACTACATTCAA
TCACCTGTGAAAAATGCCTCTGGGGAATTTTTTTGTTCAAAAAATGCTGGTAAAAGC
CTCCCACAAGATAGCGATGCAAACGGTGCATATAACATTGCCCTGAAAGGTATTCTT
CAATTACGCATGCTGTCTGAGCAGTACGACCCCAACGCGGAATCTATTAGACTTCCG
CTGATAACCAATAAAGCCTGGCTGACATTCATGCAGTCTGGCATGAAGACCTGGAA
AAATTAGGAAATCATCCTTAGCGAAAGCTAAGGATTTTTTTTATCTGAAATGTAGGG
AGACCCTCAGGTTAAATATTCACTCAGGAAGTTA
SEQ ID NO: 81
GTTTTATATCCCGGCTTGGAACACGAGCAACATAGATCCGACTACTGGATTTGTTAA
TTTATTTCATGCCCAGTATGAAAATGTAGATAAAGCGAAGAGCTTCTTTCAAAAGTT
TGATTCAATTAGTTACAACCCGAAGAAAGACTGGTTTGAGTTTGCATTCGATTATAA
AAACTTTACTAAAAAGGCTGAAGGAAGTCGTTCTATGTGGATATTATGCACACATGG
TTCCCGAATAAAGAATTTTAGAAATTCCCAGAAGAATGGTCAATGGGATTCCGAAG
AATTCGCCTTGACGGAGGCTTTTAAGTCTCTTTTTGTGCGATATGAGATAGATTATAC
CGCTGATTTGAAAACAGCTATTGTGGACGAAAAGCAAAAAGACTTCTTCGTGGATCT
TCTGAAGCTATTCAAATTGACAGTACAGATGCGCAACAGCTGGAAAGAGAAGGATT
TGGATTATCTAATCTCTCCTGTAGCAGGGGCTGATGGCCGTTTCTTCGATACAAGAG
AGGGAAATAAAAGTCTGCCTAAGGATGCAGATGCCAATGGAGCTTATAATATTGCC
CTAAAAGGACTTTGGGCTCTACGCCAGATTCGGCAAACTTCAGAAGGCGGTAAACT
CAAATTGGCGATTTCCAATAAGGAATGGCTACAGTTTGTGCAAGAGAGATCTTACG
AGAAAGACTGAGAAATCATCCTTAGCGAAAGCTAAGGATTTTTTTTATCTGAAATGT
AGGGAGACCCTCAGGTTAAATATTCACTCAGGAAGTTA
SEQ ID NO: 82
TTTTTATGTGCCTGCTGCATACACGAGCAAAATTGATCCGACCACCGGCTTTGTGAA
TATCTTTAAATTTAAAGACCTGACAGTGGACGCAAAACGTGAATTCATTAAAAAATT
TGACTCAATTCGTTATGACAGTGAAAAAAATCTGTTCTGCTTTACATTTGACTACAA
TAACTTTATTACGCAAAACACGGTCATGAGCAAATCATCGTGGAGTGTGTATACATA
CGGCGTGCGCATCAAACGTCGCTTTGTGAACGGCCGCTTCTCAAACGAAAGTGATAC
CATTGACATAACCAAAGATATGGAGAAAACGTTGGAAATGACGGACATTAACTGGC
GCGATGGCCACGATCTTCGTCAAGACATTATAGATTATGAAATTGTTCAGCACATAT
TCGAAATTTTCCGTTTAACAGTGCAAATGCGTAACTCCTTGTCTGAACTGGAGGACC
GTGATTACGATCGTCTCATTTCACCTGTACTGAACGAAAATAACATTTTTTATGACA
GCGCGAAAGCGGGGGATGCACTTCCTAAGGATGCCGATGCAAATGGTGCGTATTGT
ATTGCATTAAAAGGGTTATATGAAATTAAACAAATTACCGAAAATTGGAAAGAAGA
TGGTAAATTTTCGCGCGATAAACTCAAAATCAGCAATAAAGATTGGTTCGACTTTAT
CCAGAATAAGCGCTATCTCTAAGAAATCATCCTTAGCGAAAGCTAAGGATTTTTTTT
ATCTGAAATGTAGGGAGACCCTCAGGTTAAATATTCACTCAGGAAGTTA
SEQ ID NO: 83
ATCGACCCTACAACCGGCTTCGTCAATTACTTCTATACTAAATATGAAAACGTCGAC
AAAGCAAAAGCATTCTTTGAAAAGTTCGAAGCAATACGTTTTAACGCTGAGAAAAA
ATATTTCGAGTTCGAAGTCAAGAAATACTCAGACTTTAACCCCAAAGCTGAGGGCA
CACAGCAAGCGTGGACAATCTGCACCTACGGCGAGCGCATCGAAACGAAGCGTCAA
AAAGATCAGAATAACAAATTTGTTTCAACACCTATCAACCTGACCGAGAAGATTGA
AGACTTCTTAGGTAAAAATCAGATTGTTTATGGCGACGGTAACTGTATAAAATCTCA
AATAGCCTCAAAGGATGATAAAGCATTTTTCGAAACATTATTATATTGGTTCAAAAT
GACACTGCAGATGCGCAATAGTGAGACGCGTACAGATATTGATTATCTTATCAGCCC
GGTCATGAACGACAACGGTACTTTTTACAACTCCAGAGACTATGAAAAACTTGAGA
ATCCAACTCTCCCCAAAGATGCTGATGCGAACGGTGCTTATCACATCGCGAAAAAA
GGTCTGATGCTGCTGAACAAAATCGACCAAGCCGATCTGACTAAGAAAGTTGACCT
AAGCATTTCAAATCGGGACTGGTTACAGTTTGTTCAAAAGAACAAATGA
GAAATCATCCTTAGCGAAAGCTAAGGATTTTTTTTATCTGAAATGTAGGGAGACCCT
CAGGTTAAATATTCACTCAGGAAGTTA
SEQ ID NO: 84
TCTACACCCAGGCGTCTTACACCTCTAAATCTGACCCGGTTACCGGTTGGCGTCCGC
ACCTGTACCTGAAATACTTCTCTGCGAAAAAAGCGAAAGACGACATCGCGAAATTC
ACCAAAATCGAATTCGTTAACGACCGTTTCGAACTGACCTACGACATCAAAGACTTC
CAGCAGGCGAAAGAATACCCGAACAAAACCGTTTGGAAAGTTTGCTCTAACGTTGA
ACGTTTCCGTTGGGACAAAAACCTGAACCAGAACAAAGGTGGTTACACCCACTACA
CCAACATCACCGAAAACATCCAGGAACTGTTCACCAAATACGGTATCGACATCACC
AAAGACCTGCTGACCCAGATCTCTACCATCGACGAAAAACAGAACACCTCTTTCTTC
CGTGACTTCATCTTCTACTTCAACCTGATCTGCCAGATCCGTAACACCGACGACTCT
GAAATCGCGAAAAAAAACGGTAAAGACGACTTCATCCTGTCTCCGGTTGAACCGTT
CTTCGACTCTCGTAAAGACAACGGTAACAAACTGCCGGAAAACGGTGACGACAACG
GTGCGTACAACATCGCGCGTAAAGGTATCGTTATCCTGAACAAAATCTCTCAGTACT
CTGAAAAAAACGAAAACTGCGAAAAAATGAAATGGGGTGACCTGTACGTTTCTAAC
ATCGACTGGGACAACTTCGTTGAAATCATCCTTAGCGAAAGCTAAGGATTTTTTTTA
TCTGAAATGTAGGGAGACCCTCAGGTTAAATATTCACTCAGGAAGTTA
SEQ ID NO: 85
TCTACACCCAGGCGTCTTACACCTCTAAATCTGACCCGGTTACCGGTTGGCGTCCGC
ACCTGTACCTGAAATACTTCTCTGCGAAAAAAGCGAAAGACGACATCGCGAAATTC
ACCAAAATCGAATTCGTTAACGACCGTTTCGAACTGACCTACGACATCAAAGACTTC
CAGCAGGCGAAAGAATACCCGAACAAAACCGTTTGGAAAGTTTGCTCTAACGTTGA
ACGTTTCCGTTGGGACAAAAACCTGAACCAGAACAAAGGTGGTTACACCCACTACA
CCAACATCACCGAAAACATCCAGGAACTGTTCACCAAATACGGTATCGACATCACC
AAAGACCTGCTGACCCAGATCTCTACCATCGACGAAAAACAGAACACCTCTTTCTTC
CGTGACTTCATCTTCTACTTCAACCTGATCTGCCAGATCCGTAACACCGACGACTCT
GAAATCGCGAAAAAAAACGGTAAAGACGACTTCATCCTGTCTCCGGTTGAACCGTT
CTTCGACTCTCGTAAAGACAACGGTAACAAACTGCCGGAAAACGGTGACGACAACG
GTGCGTACAACATCGCGCGTAAAGGTATCGTTATCCTGAACAAAATCTCTCAGTACT
CTGAAAAAAACGAAAACTGCGAAAAAATGAAATGGGGTGACCTGTACGTTTCTAAC
ATCGACTGGGACAACTTCGTTGAAATCATCCTTAGCGAAAGCTAAGGATTTTTTTTA
TCTGAAATGTAGGGAGACCCTCAGGTTAAATATTCACTCAGGAAGTTA
SEQ ID NO: 86
GTAGAGTTACAAGGTTACAAGATTGATTGGACATACATTAGCGAAAAAGACA
TTGATCTGCTGCAGGAAAAAGGTCAACTGTATCTGTTCCAGATATATAACAA
AGATTTTTCGAAAAAATCAACCGGGAATGACAACCTTCACACCATGTACCTG
AAAAATCTTTTCTCAGAAGAAAATCTTAAGGATATCGTCCTGAAACTTAACG
GCGAAGCGGAAATCTTCTTCAGGAAGAGCAGCATAAAGAACCCAATCATTCA
TAAAAAAGGCTCGATTTTAGTCAACCGTACCTACGAAGCAGAAGAAAAAGA
CCAGTTTGGCAACATTCAAATTGTGCGTAAAAATATTCCGGAAAACATTTATC
AGGAGCTGTACAAATACTTCAACGATAAAAGCGACAAAGAGCTGTCTGATGA
AGCAGCCAAACTGAAGAATGTAGTGGGACACCACGAGGCAGCGACGAATAT
AGTCAAGGACTATCGCTACACGTATGATAAATACTTCCTTCATATGCCTATTA
CGATCAATTTCAAAGCCAATAAAACGGGTTTTATTAATGATAGGATCTTACA
GTATATCGCTAAAGAAAAAGACTTACATGTGATCGGCATTGATCGGGGCGAG
CGTAACCTGATCTACGTGTCCGTGATTGATACTTGTGGTAATATAGTTGAACA
GAAAAGCTTTAACATTGTAAACGGCTACGACTATCAGATAAAACTGAAACAA
CAGGAGGGCGCTAGACAGATTGCGCGGAAAGAATGGAAAGAAATTGGTAAA
ATTAAAGAGATCAAAGAGGGCTACCTGAGCTTAGTAATCCACGAGATCTCTA
AAATGGTAATCAAATACAATGCAATTATAGCGATGGAGGATTTGTCTTATGG
TTTTAAAAAAGGGCGCTTTAAGGTCGAACGGCAAGTTTACCAGAAATTTGAA
ACCATGCTCATCAATAAACTCAACTATCTGGTATTTAAAGATATTTCGATTAC
CGAGAATGGCGGTCTCCTGAAAGGTTATCAGCTGACATACATTCCTGATAAA
CTTAAAAACGTGGGTCATCAGTGCGGCTGCATTTTTTATGTGCCTGCTGCATA
TCACGAGC

What is claimed is:

1. A method for generating a library of chimeric nuclease nucleic acid sequences, said method comprising:
a. providing a plurality of at least a first and second nuclease nucleic acid comprising at least two domain sequences;
b. replacing at least one of the two domain sequences of the first nuclease nucleic acid sequence with the corresponding domain sequence of the second nuclease nucleic acid sequence, thereby generating the library of chimeric nuclease nucleic acid sequences.

2. The method of claim 1, wherein the first and second nucleic acid sequence comprise at least three domain sequences, and wherein two or more domain sequences of the first nuclease nucleic acid are replaced by the corresponding domain sequences of the second nuclease nucleic acid sequence, thereby generating the library of chimeric nuclease nucleic acid sequences.

3. The method of claim 1, wherein replacing comprises PCR amplifying the domain sequences.

4. The method of claim 3, wherein replacing further comprises performing an in vitro assembly method.

5. The method of claim 1, wherein the chimeric nuclease is a chimeric nucleic acid-guided nuclease.

6. The method of claim 5, wherein the chimeric nucleic acid-guided nuclease is capable of targeting a target nucleic acid sequence.

7. The method of claim 5, wherein one or more of the domain sequences encodes a globular domain.

8. The method of claim 5, wherein one or more domain sequences encodes a modular looped out helical domain capable of mediating DNA binding.

9. The method of claim 5, wherein one or more domain sequences encodes a globular domain capable of interacting with a displaced DNA sequence complementary to the target DNA sequence.

10. The method of claim 1, wherein at least one nuclease sequence is from a nuclease of the Cpf1 family.

11. A method for generating a library of chimeric nuclease nucleic acid sequences, said method comprising:
a. providing a plurality of at least three nuclease nucleic acids, the nucleases comprising at least three domain sequences;
b. replacing at least one of the three domain sequences of the first nuclease nucleic acid sequence with the corresponding domain sequence of the second nuclease nucleic acid sequence, and replacing at least one of the other three domain sequences of the first nuclease nucleic acid sequence with the corresponding domain sequence of the third nuclease nucleic acid sequence, thereby generating the library of chimeric nuclease nucleic acid sequences.

12. The method of claim 11, wherein replacing comprises PCR amplifying the domain sequences.

13. The method of claim 12, wherein replacing further comprises performing an in vitro assembly method.

14. The method of claim 11, wherein the chimeric nuclease is a chimeric nucleic acid-guided nuclease.

15. The method of claim 14, wherein the chimeric nucleic acid-guided nuclease is capable of targeting a target nucleic acid sequence.

16. The method of claim 14, wherein one or more of the domain sequences encodes a globular domain.

17. The method of claim 14, wherein one or more domain sequences encodes a modular looped out helical domain capable of mediating DNA binding.

18. The method of claim 14, wherein one or more domain sequences encodes a globular domain capable of interacting with a displaced DNA sequence complementary to the target DNA sequence.

19. The method of claim 11, wherein at least one nuclease nucleic acid is from the Cpf1 family.

20. The method of claim 11, wherein at least two nuclease nucleic acids are from the Cpf1 family.
